(19) United States
(12) Patent Application Publication
(10) Pub. No.: US 2009/0075968 A1
(43) Pub. Date: Mar. 19, 2009

(54) CETP inhibitors

(76) Inventors: Junichi Sakaki, Ibaraki (JP); Masashi Kishida, Ibaraki (JP); Naoko Matsuura, Ibaraki (JP); Ichiro Umemura, Ibaraki (JP); Eiji Kawahara, Ibaraki (JP); Ken Yamada, Ibaraki (JP); Kazuhide Konishi, Ibaraki (JP); Yuki Iwaki, Ibaraki (JP); Hidetomo Imase, Ibaraki (JP); Takahiro Miyake, Ibaraki (JP)

Correspondence Address: NOVARTIS INSTITUTES FOR BIOMEDICAL RESEARCH, INC., 400 TECHNOLOGY SQUARE, CAMBRIDGE, MA, 02139, US

(21) Appl. No.: 12/159,502

(22) PCT Filed: Dec. 27, 2006
(86) PCT No.: PCT/EP06/12540

§ 371 Date: Jun. 27, 2008

(30) Foreign Application Priority Data
Dec. 29, 2005 (EP) 05028617.8
May 10, 2006 (GB) 0609270.4

(51) Int. Cl. A61K 31/4439; C07D 401/12; C07D 213/26; C07D 401/04; A61K 31/506; A61K 31/55; A61P 3/00; A61P 9/00; A61K 31/397; A61K 31/5377; A61K 31/444; C07D 413/14; C07D 401/14

(52) U.S. Cl. 514/210.2; 546/268.4; 546/275.4; 546/272.4; 546/304; 546/309; 546/312; 544/333; 544/124; 544/336; 540/597; 544/114; 514/340; 514/333; 514/256; 514/235.5; 514/217.04

(57) ABSTRACT

The present invention relates to a compound of formula

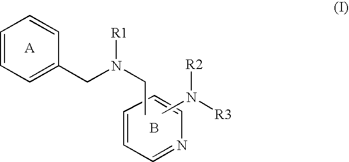

or a pharmaceutically acceptable salt thereof, wherein the variables are as defined.

The present invention relates to a novel compound of formula I

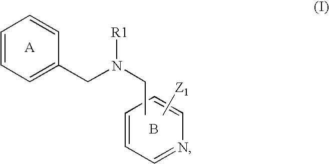

wherein
Z₁ is selected from the group consisting of —N(R₂)(R₃), —CN, —OR′, —COR′, —C(═O)—O—R′, —C(═O)—NR₂R₃, —S(O)ₘR′, —S(O)ₘ—N(R₂)(R₃) and —NR′—S(O)ₘ—N(R₂)(R₃), m being in each case the integer 0, 1 or 2, or Z₁ is Z;
R₁ is the element —C(═O)—R′, —C(═O)—O—R′, —C(═O)—NR₂R₃, —S(O)ₘ—R′, —S(O)ₘ—N(R₂)(R₃), m being in each case the integer 0, 1 or 2, or R₁ is Z;
wherein, in each case, independently of one another,
Z is selected from the group consisting of (i) unsubstituted or substituted monocyclic cycloalkyl or unsubstituted or substituted monocyclic cycloalkenyl, (ii) unsubstituted or substituted carbocyclic aromatic radical or unsubstituted or substituted heterocyclic radical;
R′, independently, represents hydrogen, alkyl, haloalkyl, unsubstituted or substituted cycloalkyl, unsubstituted or substituted cycloalkenyl, in the cycloalkyl moiety unsubstituted or substituted cycloalkyl-alkyl, in the cycloalkenyl moiety unsubstituted or substituted cycloalkenyl-alkyl, unsubstituted or substituted carbocyclic aromatic radical, unsubstituted or substituted heterocyclic radical or in the aryl moiety unsubstituted or substituted aralkyl;
R₂ and R₃, independently of one another, represents hydrogen, alkyl, alkyl which is substituted by one or more substituents selected from the group consisting of halogen, hydroxy, —N(R₂)(R₃), —C(═O)—O—R′, —C(═O)—NR₂R₃, —S(O)ₘ—R′, —S(O)ₘ—N(R₂)(R₃), unsubstituted or substituted cycloalkyl, unsubstituted or substituted cycloalkenyl, and unsubstituted or substituted heterocyclic radical; or R₂ and R₃, independently of one another, represents unsubstituted or substituted cycloalkyl, unsubstituted or substituted cycloalkenyl, or unsubstituted or substituted carbocyclic aromatic radical, of unsubstituted or substituted heterocyclic radical; and
R₂ and R₃ together are unsubstituted or substituted alkylene or unsubstituted or substituted alkylene that is interrupted by O, NR″ or S; R″ being R′ or —C(═O)—O—R′; and
wherein substituted cycloalkyl or substituted cycloalkenyl each of which substituted is by one or more substituents selected from the group consisting of alkyl, of alkoxy, of —C(═O)—O—R′, of —C(═O)—NR₂R₃, of —N(R₂)(R₃), of cycloalkyl-alkyl, of unsubstituted or substituted carbocyclic aromatic radical, of unsubstituted or substituted heterocyclic radical, of in the aryl moiety unsubstituted or substituted aralkyl, and of in the heterocyclyl moiety unsubstituted or substituted heterocyclyl-alkyl; and
wherein a carbocyclic aromatic radical or a heterocyclic aromatic radical or a heterocyclic radical, in the aryl moiety unsubstituted or substituted aralkyl, in the heterocyclyl moiety unsubstituted or substituted heterocyclyl-alkyl, or the rings A and B, independently of one another, are unsubstituted or substituted by one or more substituents selected from the group consisting of halogen, NO₂, CN, OH, alkyl, alkoxy-alkyl, hydroxy-alkyl, halo-alkyl, alkoxy, alkoxy-alkoxy, haloalkoxy, —C(═O)—R′, —C(═O)—O—R′, —N(R₂)(R₃), —C(═O)—NR₂R₃, —S(O)ₘ—R′, —S(O)ₘ—N(R₂)(R₃), —NR′—S(O)ₘ—N(R₂)(R₃) and alkanoyl(oxy), m being in each case the integer 0, 1 or 2; and unsubstituted or substituted cycloalkyl, unsubstituted or substituted cycloalkenyl; in the aryl moiety unsubstituted or substituted aralkyl and in the heterocyclyl moiety unsubstituted or substituted heterocyclyl-alkyl;
in free form or in salt form; to a process for the preparation of these compounds, to the use of these compounds and to pharmaceutical preparations containing such a compound I in free form or in the form of a pharmaceutically acceptable salt.

The compounds (I) can be present as salts, in particular pharmaceutically acceptable salts. If the compounds I have, for example, at least one basic centre, they can form acid addition salts. The compounds I having at least one acid group can also form salts with bases. Salts which are unsuitable for pharmaceutical uses but which can be employed, for example, for the isolation or purification of free compounds (I) or their pharmaceutically acceptable salts, are also included. In view of the close relationship between the novel compound in the free form and in the form of its salts, in the preceding text and below the free compound or its salts may correspondingly and advantageously also be understood as meaning the corresponding salts or the free compound.

The general definitions used above and below, unless defined differently, have the following meanings:

If not defined otherwise, alkyl being a radical or part of a radical is especially C₁-C₇-alkyl, preferably C₁-C₄-alkyl.

A carbocyclic aromatic radical is, in particular, phenyl, biphenylyl or naphthyl.

Biphenylyl is, for example, 4-biphenylyl, and also a 2- or 3-biphenylyl.

Naphthyl is 1- or 2-naphthyl.

A heterocyclic radical is, in particular, heteroaryl is a 5-14 membered monocyclic- or bicyclic- or fused polycyclic-ring system, having 1 to 8 heteroatoms selected from N, O or S. Preferably, the heteroaryl is a 5-10 membered ring system. A heterocyclic aromatic radical group may be mono-, bi-, tri-, or polycyclic, preferably mono-, bi-, or tricyclic, more preferably mono- or bicyclic. A heterocyclic radical can also be a partially or fully saturated heteroaryl.

A heterocyclic radical is, in particular, an unsubstituted or substituted 5- to 6-membered heterocyclic ring having 1, 2, 3 or 4 hetero atoms selected from the group consisting of N, S and O.

A heterocyclic radical is, in particular, an unsubstituted or substituted benzofused heterocyclic ring having 1 or 2 hetero atoms selected from the group consisting of N, S and O, and the heterocyclic ring being saturated or having 1 or 2 double bonds

Typical heteroaryl groups include 2- or 3-thienyl, 2- or 3-furyl, 2- or 3-pyrrolyl, 2-, 4-, or 5-imidazolyl, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 3- or 5-1,2,4-triazolyl, 4- or 5-1,2,3-triazolyl, tetrazolyl, 2-, 3-, or 4-pyridyl, 3- or 4-pyridazinyl, 3-, 4-, or 5-pyrazinyl, 2-pyrazinyl, 2-, 4-, or 5-pyrimidinyl.

A heterocyclic aromatic radical is also a group in which a heteroaromatic ring is fused to one or more aryl, cycloaliphatic, or heterocyclyl rings, where the radical or point of attachment is on the heteroaromatic ring. Nonlimiting examples include but are not limited to 1-, 2-, 3-, 5-, 6-, 7-, or 8-indolizinyl, 1-, 3-, 4-, 5-, 6-, or 7-isoindolyl, 2-, 3-, 4-, 5-, 6-, or 7-indolyl, 2-, 3-, 4-, 5-, 6-, or 7-indazolyl, 2-, 4-, 5-, 6-, 7-, or 8-purinyl, 1-, 2-, 3-, 4-, 6-, 7-, 8-, or 9-quinolizinyl, 2-, 3-, 4-, 5-, 6-, 7-, or 8-quinoliyl, 1-, 3-, 4-, 5-, 6-, 7-, or 8-isoquinoliyl, 1-, 4-, 5-, 6-, 7-, or 8-phthalazinyl, 2-, 3-, 4-, 5-, or 6-naphthyridinyl, 2-, 3-, 5-, 6-, 7-, or 8-quinazolinyl, 3-, 4-, 5-, 6-, 7-, or 8-cinnolinyl, 2-, 4-, 6-, or 7-pteridinyl, 1-, 2-, 3-, 4-, 5-, 6-, 7-, or 8-4-aH-carbazolyl, 1-, 2-, 3-, 4-, 5-, 6-, 7-, or 8-carbazolyl, 1-, 3-, 4-, 5-, 6-, 7-, 8-, or 9-carbolinyl, 1-, 2-, 3-, 4-, 6-, 7-, 8-, 9-, or 10-phenanthridinyl, 1-, 2-, 3-, 4-, 5-, 6-, 7-, 8-, or 9-acridinyl, 1-, 2-, 4-, 5-, 6-, 7-, 8-, or 9-perimidinyl, 2-, 3-, 4-, 5-, 6-, 8-, 9-, or 10-phenathrolinyl, 1-, 2-, 3-, 4-, 6-, 7-, 8-, or 9-phenazinyl, 1-, 2-, 3-, 4-, 6-, 7-, 8-, 9-, or 10-phenothiazinyl, 1-, 2-, 3-, 4-, 6-, 7-, 8-, 9-, or 10-phenoxazinyl, 2-, 3-, 4-, 5-, 6-, or I-, 3-, 4-, 5-, 6-, 7-, 8-, 9-, or 10-benzisoqinolinyl, 2-, 3-, 4-, or thieno[2,3-b]furanyl, 2-, 3-, 5-, 6-, 7-, 8-, 9-, 10-, or 11-7H-pyrazino[2,3-c]carbazolyl, 2-, 3-, 5-, 6-, or 7-2H-furo[3,2-b]-pyranyl, 2-, 3-, 4-, 5-, 7-, or 8-5H-pyrido[2,3-d]-o-oxazinyl, 1-, 3-, or 5-1H-pyrazolo[4,3-d]-oxazolyl, 2-, 4-, or 54H-imidazo[4,5-d]thiazolyl, 3-, 5-, or 8-pyrazino[2,3-d]pyridazinyl, 2-, 3-, 5-, or 6-imidazo[2,1-b]thiazolyl, 1-, 3-, 6-, 7-, 8-, or 9-furo[3,4-c]cinnolinyl, 1-, 2-, 3-, 4-, 5-, 6-, 8-, 9-, 10, or 11-4H-pyrido[2,3-c]carbazolyl, 2-, 3-, 6-, or 7-imidazo[1,2-b][1,2,4]triazinyl, 7-benzo[b]thienyl, 2-, 4-, 5-, 6-, or 7-benzoxazolyl, 2-, 4-, 5-, 6-, or 7-benzimidazolyl, 2-, 4-, 4-, 5-, 6-, or 7-benzothiazolyl, 1-, 2-, 4-, 5-, 6-, 7-, 8-, or 9-benzoxapinyl, 2-, 4-, 5-, 6-, 7-, or 8-benzoxazinyl, 1-, 2-, 3-, 5-, 6-, 7-, 8-, 9-, 10-, or 11-1H-pyrrolo[1,2-b][2]benzazapinyl. Typical fused heteroary groups include, but are not limited to 2-, 3-, 4-, 5-, 6-, 7-, or 8-quinolinyl, 1-, 3-, 4-, 5-, 6-, 7-, or 8-isoquinolinyl, 2-, 3-, 4-, 5-, 6-, or 7-indolyl, 2-, 3-, 4-, 5-, 6-, or 7-benzo[b]thienyl, 2-, 4-, 5-, 6-, or 7-benzoxazolyl, 2-, 4-, 5-, 6-, or 7-benzimidazolyl, 2-, 4-, 5-, 6-, or 7-benzothiazolyl.

An appropriate 5- or 6-membered and monocyclic radical which has up to four identical or different hetero atoms, such as nitrogen, oxygen or sulfur atoms, preferably one, two, three or four nitrogen atoms, an oxygen atom or a sulfur atom. Appropriate 5-membered heteroaryl radicals are, for example, monoaza-, diaza-, triaza-, tetraaza-, monooxa- or monothia-cyclic aryl radicals, such as pyrrolyl, pyrazolyl, imidazolyl, triazolyl, tetrazolyl, furyl and thienyl, while suitable appropriate 6-membered radicals are in particular pyridyl and pyrimidyl. Appropriate aromatic radicals are radicals which may be monosubstituted or polysubstituted, for example di- or trisubstituted, for example by identical or different radicals.

Pyrrolyl is, for example, 2- or 3-pyrrolyl. Pyrazolyl is 3- or 4-pyrazolyl. Imidazolyl is 2- or 4-imidazolyl. Triazolyl is, for example, 1,3,5-1H-triazol-2-yl or 1,3,4-triazol-2-yl. Tetrazolyl is, for example, 1,2,3,4-tetrazol-5-yl. Furyl is 2- or 3-furyl and thienyl is 2- or 3-thienyl, while suitable pyridyl is 2-, 3- or 4-pyridyl.

Preferred is 1,2,3,4-tetrazol-5-yl or 1,3,4-triazol-2-yl.

A benzofused heterocyclic ring having 1 or 2 hetero atoms selected from the group consisting of N, S and O, and the heterocyclic ring being saturated or having 1 or 2 double bonds is, for example, indole, quinoline, indoline or tetrahydroisoquinoline.

A 5- to 6-membered heterocyclic ring having 1, 2 or 3 hetero atoms selected from the group consisting of N, S and O is in particular a substituted tetrazole, substituted triazole, such as methyltriazole, a substituted pyrimidine or a substituted pyrazole, such as methylpyrazole. Further ones comprise substituted pyridine, substituted-triazine, imidazole, oxazole, thiazole. A preferred substituent is alkyl, such as methyl.

A 5-14 membered monocyclic- or bicyclic- or fused polycyclic-ring system, having 1 to 8 heteroatoms selected from N, O or S, is also partially or fully saturated.

Preferred is a partially or fully saturated heteroaryl 5- to 6-membered heterocyclic ring having 1, 2, 3 or 4 hetero atoms selected from the group consisting of N, S and O is, for example, a pyrroline radical, pyrrolidine radical, a dihydro- or a tetrahydro-thienyl radical, a dihydro- or a tetrahydro-furan radical, a dihydro- or tetrahydro-pyridine radical, an imidazoline or imidazolidine radical, a pyrazoline or pyrazolidine radical, a thiazoline or thiazolidine radical, an oxazoline or oxazolidine radical, a dihydro- or tetrahydro-pyridine or piperidine radical, or a dihydro- or tetrahydro-pyrane radical. Preferred 5- to 6-membered N-heterocyclic radicals are, for example, bonded via the N-atom, especially a pyrrolidin-1-yl radical.

A heterocyclic radical is unsubstituted or substituted by one or more, for example two or three, substituents. Preferred are corresponding C-substituted radicals.

Cycloalkyl is, for example, C₃-C₇-cycloalkyl and is, for example, cyclopropyl, cyclobutyl, cyclopentyl, cyclohexyl and cycloheptyl. Cyclopentyl and cyclohexyl are preferred. Cycloalkenyl is, for example, C₃-C₇-cycloalkenyl and is, for example, cyclopropenyl, cyclobutenyl, cyclopentenyl, cyclohexenyl and cycloheptenyl. Cyclopentenyl and cyclohexenyl are preferred.

Alkyl is especially C₁-C₇-alkyl and is, for example methyl, ethyl, n-propyl, isopropyl, n-butyl, isobutyl, sec-butyl, tert-butyl, and also includes corresponding pentyl, hexyl and heptyl radicals. C₁-C₄alkyl is preferred.

Halogen is in particular halogen of atomic number not more than 35, such as fluoro, chloro or bromo, and also includes iodo. Preferred is chloro.

Halo-alkyl is, for example, halo-C₁-C₇alkyl and is in particular halo-C₁-C₄alkyl, such as trifluoromethyl, 1,1,2-trifluoro-2-chloroethyl or chloromethyl.

Aralkyl is for example, carboxyclic aryl-alkyl, preferably, phenyl-C₁-C₄-alkyl, such as benzyl or 2-phenethyl.

Alkoxy is, for example, C₁-C₇-alkoxy and is, for example, methoxy, ethoxy, n-propyloxy, isopropyloxy, n-butyloxy, isobutyloxy, sec-butyloxy, tert-butyloxy and also includes corresponding pentyloxy, hexyloxy and heptyloxy radicals. C₁-C₄alkoxy is preferred.

Alkanoyl is, for example, C₂-C₇alkanoyl and is, for example, acetyl, propionyl, butyryl, isobutyryl or pivaloyl. C₂-C₅-Alkanoyl is preferred, especially acetyl.

Substituted alkylene is especially substituted C₂-C₇-alkylene or substituted C₂-C₇-alkylene which is interrupted by O, NR″ or S each of which can be substituted, for example, by C₁-C₇-alkyl, by C₁-C₇-alkoxy-C₁-C₇-alkyl, by carboxy, by C₁-C₇-alkoxy-carbonyl, by C₃-C₇-cycloalkyl or by C₃-C₇-cycloalkyl which is either annelated or attached to said alkylene in spiro form.

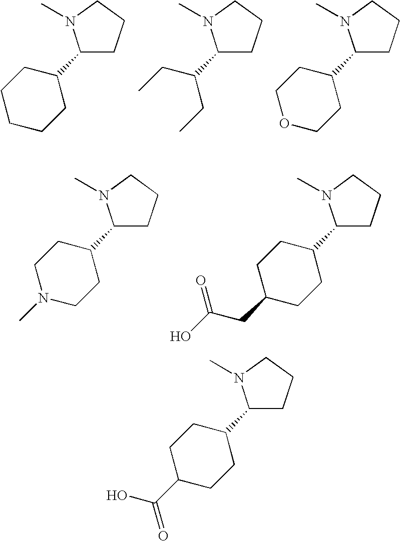

With respect to a compound of formula (R′), the general definitions used below, unless defined differently, have the following meanings:

A carbocyclic or heterocyclic aromatic radical is, in particular phenyl, biphenylyl or naphthyl, in particular an appropriate 5- or 6-membered and monocyclic radical which has up to four identical or different hetero atoms, such as nitrogen, oxygen or sulfur atoms, preferably one, two, three or four nitrogen atoms, an oxygen atom or a sulfur atom. Appropriate 5-membered heteroaryl radicals are, for example, monoaza-, diaza-, triaza-, tetraaza-, monooxa- or monothia-cyclic aryl radicals, such as pyrrolyl, pyrazolyl, imidazolyl, triazolyl, tetrazolyl, furyl and thienyl, while suitable appropriate 6-membered radicals are in particular pyridyl. Appropriate aromatic radicals are radicals which may be monosubstituted or polysubstituted, for example di- or trisubstituted, for example by identical or different radicals.

Biphenylyl is, for example, 4-biphenylyl, also a 2- or 3-biphenylyl.

Naphthyl is 1- or 2-naphthyl.

Pyrrolyl is, for example, 2- or 3-pyrrolyl. Pyrazolyl is 3- or 4-pyrazolyl. Imidazolyl is 2- or 4-imidazolyl. Triazolyl is, for example, 1,3,5-1H-triazol-2-yl or 1,3,4-triazol-2-yl. Tetrazolyl is, for example, 1,2,3,4-tetrazol-5-yl. Furyl is 2- or 3-furyl and thienyl is 2- or 3-thienyl, while suitable pyridyl is 2-, 3- or 4-pyridyl.

Preferred is

Alkoxy is preferably C₁-C₇-Alkoxy and is, for example, methoxy, ethoxy, n-propyloxy, isopropyloxy, n-butyloxy, isobutyloxy, sec-butyloxy, tert-butyloxy and also includes corresponding pentyloxy, hexyloxy and heptyloxy radicals. C₁-C₄alkoxy is preferred.

Cycloalkyl is preferably C₃-C₇-Cycloalkyl and is, for example, cyclopropyl, cyclobutyl, cyclopentyl, cyclohexyl and cycloheptyl. Cyclopentyl and cyclohexyl are preferred.

Cycloalkenyl is preferably C₃-C₇-Cycloalkyl having one, two or three double bonds and is, for example, cyclopropenyl, cyclobutenyl, cyclohexenyl or cyclohexadienyl.

Alkyl is preferably C₁-C₇-Alkyl and is, for example methyl, ethyl, n-propyl, isopropyl, n-butyl, isobutyl, sec-butyl, tert-butyl, and also includes corresponding pentyl, hexyl and heptyl radicals. C₁-C₄alkyl is preferred.

### I

C₂-C₈-Alkylene is straight-chain or branched and is in particular ethylene, propylene and butylene and also 1,2-propylene, 2-methyl-1,3-propylene and 2,2-dimethyl-1,3-propylene. C₂-C₅-Alkylene is preferred.

Halogen is in particular halogen of atomic number not more than 35, such as fluoro, chloro or bromo, and also includes iodo. Preferred is chloro.

Halo-alkyl is preferably Halo-C₁-C₇alkyl and is in particular halo-C₁-C₄alkyl, such as trifluoromethyl, 1,1,2-trifluoro-2-chloroethyl or chloromethyl.

Alkanoyl is preferably C₂-C₇-Alkanoyl and is, for example, acetyl, propionyl, butyryl, isobutyryl or pivaloyl. C₂-C₅-alkanoyl is preferred, especially acetyl.

Two substituents together with the two carbon atoms to which they are attached can form a 5- or 6-membered ring.

Extensive pharmacological investigations have shown that the compounds I and their pharmaceutically acceptable salts, for example, have pronounced selectivity in inhibiting CETP (cholesteryl ester transfer protein). CETP is involved in the metabolism of any lipoprotein in living organisms, and has a major role in the reverse cholesterol transfer system. Namely, CETP has drawn attention as a mechanism for preventing accumulation of cholesterol in peripheral cells and preventing arteriosclerosis. In fact, with regard to HDL having an important role in this reverse cholesterol transfer system, a number of epidemiological researches have shown that a decrease in CE (cholesteryl ester) of HDL in blood is one of the risk factors of coronary artery diseases. It has been also clarified that the CETP activity varies depending on the animal species, wherein arteriosclerosis due to cholesterol-loading is hardly induced in animals with lower activity, and in reverse, easily induced in animals with higher activity, and that hyper-HDL-emia and hypo-LDL (low density lipoprotein)-emia are induced in the case of CETP deficiency, thus rendering the development of arteriosclerosis difficult, which in turn led to the recognition of the significance of blood HDL, as well as significance of CETP that mediates transfer of CE in HDL into blood LDL. While many attempts have been made in recent years to develop a drug that inhibits such activity of CETP, a compound having a satisfactory activity has not been developed yet.

The CETP inhibitory effect of the compounds of the present invention can be demonstrated by using test models know by a person skilled in the pertinent art, for example, following test models:

### (1) CETP In Vitro Assay:

CETP Activity Kit (#RB-RPAK) was purchased from Roar Biochemical, Inc. (New York, N.Y., USA). To each well of a 96-well NBS half-area plate (costar #3686), 1.2 ng/well of the donor solution, 1 μL of the acceptor solution and 5 μL compound solution diluted in 100% DMSO were added in a 38 μL of buffer containing 10 mM Tris, 150 mM NaCl and 2 mM EDTA, pH 7.4. Then, the plate was sealed with Themowell™ Sealers (costar #6524) and followed by a mixing on a plate shaker by MICROPLATE MIXER MPX-96 (IWAKI) at power 3 for 10 sec at room temperature. After 10-min incubation at 37° C., the reaction was started by adding 5 μL of rhCETP solution (Cardiovascular Target, New York, N.Y., USA) and mixed on the plate shaker for 10 sec, then the fluorescence intensity at 0 min was measured by a ARVO SX (Perkin Elmerr, USA) at excitation wavelength of 465 nm and emission wavelength of 535 nm. After 120 min-incubation at 37° C., fluorescence intensity was measured again. The inhibition of rhCETP activity by a compound was calculated by the following calculation. Inhibition %={1−(F120−F0)/(f120−f0)}×100 F: measured fluorescence intensity with compound at 0 or 120 min. f: measured fluorescence intensity of without compound at 0 or 120 min.

The IC₅₀ values are determined from the dose-effect curve by Origin software. IC₅₀ values, especially from about 0.1 nM to about 50 μM, are determined for the compounds of the present invention or a pharmaceutically acceptable salt thereof.

### (2) Effects on Plasma HDL Levels in Hamster:

Effects of compounds on HDL-cholesterol level in hamsters are investigated by the method reported previously with some modifications (Eur, J. Phamacol, 466 (2003) 147-154). In brief, male Syrian hamsters (10-11 week old age, SLC, Shizuoka, Japan) are fed a high cholesterol diet for two weeks. Then, the animals are dosed singly with the compound suspended with carboxyl methyl cellulose solution. HDL-cholesterol levels are measured by using commercially available kit (Wako Pure Chemical, Japan) after the precipitation of apolipoprotein B (apoB)-containing lipoproteins with 13% polyethylene glycol 6000.

(3) Preparation of Human Pro-Apolipoprotein AI (pro-apoAI)

The cDNA of human pro-apoAI (NCBI accession number: NM_{—}000039) is cloned from human liver Quick-Clone™ cDNA (Clontech, CA) and inserted to a pET28a vector (Novagen, Germany) for bacterial expression. Expressed protein as a fusion protein with 6×His-tag at N-terminus in BL-21 Gold (DE3) (Strategene, CA) is purified using HiTrap Chelating (GE Healthcare, Conn.).

### (4) Preparation of Donor Microemulsion

Pro-apoAI containing microemulsion as a donor particle is prepared following previous reports (J. Biol. Chem., 280:14918-22). Glyceryl trioleate (62.5 ng, Sigma, Mo.), 3-sn-phosphatidylcholine (583 ng, Wako Pure Chemical Industries, Japan), and cholesteryl BODIPY® FL C₁₂ (250 ng, Invitrogen, Calif.) are dissolved in 1 mL of chloroform. The solution is evaporated, then residual solvent is removed in vacuum for more than 1 hr. The dried lipid mixture is dissolved in 500 μL of the assay buffer (50 mM Tris-HCl (pH7.4) containing 150 mM NaCl and 2 mM EDTA) and sonicated at 50° C. with a microtip (MICROSON™ ULTRASONIC CELL DISRUPTOR, Misonix, Farmingdale, N.Y.) at output power 006 for 2 min. After sonication, the solution is cooled to 40° C., added to 100 μg of human pro-apoAI, and sonicated at output power 004 for 5 min at 40° C. The solution, BODIPY-CE microemulsion as a donor molecule is stored at 4° C. after filtration through a 0.45 μm PVDF filter.

### (5) In Vitro CETP Activity Assay in Human Plasma

Human EDTA plasma samples from healthy men are purchased from New Drug Development Research Center, Inc. Donor solution is prepared by a dilution of donor microemulsion with assay buffer. Human plasma (50 μL), assay buffer (35 μL) and test compound dissolved in dimethylsulfoxide (1 μL) are added to each well of 96 well half area black flat bottom plate. The reaction is started by the addition of donor solution (14 μL) into each well. Fluorescence intensities are measured every 30 min at 37° C. with excitation wave length of 485 nm and emission wavelength of 535 nm. The CETP activity (Fl/min) is defined as the changes of fluorescence intensity from 30 to 90 min. The IC₅₀ value is obtained by the logistic equation (Y=Bottom+(Top-Bottom)/(1+(x/IC₅₀)̂Hill slope) using Origin software, version 7.5 SR3. The compounds of formula I exhibit inhibitory activity with an IC₅₀ value in the range from approximately from 0.001 to 100 μM, especially from 0.01 to 10 μM.

The compounds of the present invention or a pharmaceutically acceptable salt thereof have superior CETP inhibitory activity in mammals (e.g., human, monkey, bovine, horse, dog, cat, rabbit, rat, mouse and the like), and can be used as CETP activity inhibitors. In addition, utilizing the superior CETP inhibitory activity of a compound of the present invention or a pharmaceutically acceptable salt thereof, the compounds of the present invention are useful as pharmaceutical agents effective for the prophylaxis or treatment of or delay progression to overt to diseases in which CETP is involved (e.g., hyperlipidemia, arteriosclerosis, atherosclerosis, peripheral vascular disease, dyslipidemia, hyperbetalipoproteinemia, hypoalphalipoproteinemia, hypercholesterolemia, hypertriglyceridemia, familial hypercholesterolemia, cardiovascular disorder, coronary heart disease, coronary artery disease, coronary vascular disease, angina, ischemia, heart ischemia, thrombosis, cardiac infarction such as myocardial infarction, stroke, peripheral vascular disease, reperfusion injury, angioplasty restenosis, hypertension, congestive heart failure, diabetes such as type II diabetes mellitus, diabetic vascular complications, obesity or endotoxemia etc.), particularly as prophylactic or therapeutic agents for hyperlipidemia or arteriosclerotic diseases.

A further aspect of the present invention is the use of a CETP inhibitor for the prophylaxis or treatment of or delay progression to overt to a disease selected from the group consisting of coronary heart disease, coronary artery disease, coronary vascular disease, myocardial infarction, stroke, peripheral vascular disease, diabetes such as type II diabetes mellitus, congestive heart failure, and reperfusion injury.

Preferred Z₁ is pyrrolidine-1-yl which is substituted by C₃-C₇-cycloalkyl or is the element —N(R₄)(R₅) and also a radical selected from

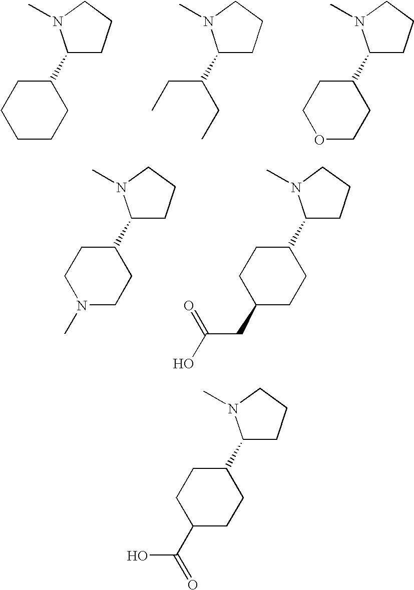

Preferred is a compound of formula (I′)

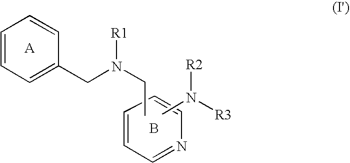

or a pharmaceutically acceptable salt thereof.

More preferred is a compound of formula (I A)

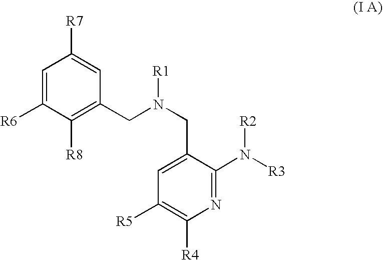

or a pharmaceutically acceptable salt thereof.

A preferred meaning of variable R₁ is represented by formulae

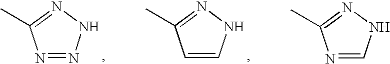

which are unsubstituted or N-substituted by C₁-C₄-alkyl, especially methyl; preferably 1-C₁-C₄-alkyl-tetrazol-3-yl, especially 1-methyl-tetrazol-3-y, or is phenyl, C₁-C₄-alkoxy-carbonyl, C₁-C₄-alkyl-carbonyl, C₁-C₄-alkyl-S(O)₂, phenyl-S(O)₂, phenyl being unsubstituted or substituted by a substituent as defined hereinbefore and hereinafter.

A preferred meaning of variable R₂ is C₁-C₄-alkyl, especially ethyl.

A preferred meaning of variable R₃ is C₅-C₆-cycloalkyl-C₁-C₄-alkyl cycloalkyl being unsubstituted or substituted by carboxy-C₁-C₄-alkyl such as carboxy-methyl, by C₁-C₄-alkoxy-carbonyl-C₁-C₄-alkyl such as ethoxy-carbonyl-methyl, by carbamoyl-C₁-C₄-alkyl, such as carbamoyl-methyl, by hydroxyl-C₁-C₄-alkyl such as 2-hydroxy-ethyl, by amino-C₁-C₄-alkyl such as 2-amino-methyl. Especially preferred is cyclohexyl, cyclohexanoyl, and acetyl.

A preferred meaning of variable R₄ is hydrogen, C₁-C₄-alkyl, halo-C₁-C₄-alkyl, especially trifluoromethyl, preferably hydrogen.

A preferred meaning of variable R₅ is halogen, C₁-C₄-alkyl, halogen, halo-C₁-C₇-alkyl, especially trifluoromethyl, most preferably trifluoromethyl.

A preferred meaning of variable R₆ is halogen, C₁-C₄-alkyl, halogen, halo-C₁-C₇-alkyl, especially trifluoromethyl, most preferably trifluoromethyl.

A preferred meaning of variable R₇ is NO₂, CN, halogen, and halo-C₁-C₇-alkyl, especially trifluoromethyl, most preferably trifluoromethyl.

A preferred meaning of R₈ is hydrogen or halogen, especially fluoro, most preferably hydrogen.

Preferred is a compound of formula (I′)

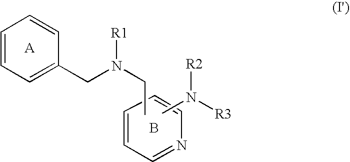

or a pharmaceutically acceptable salt thereof,
wherein R₁ is carbocyclic or heterocyclic aryl, alkoxy-CO—, cylcoalkyl-alkoxy-CO—, carbocyclic aryl-alkoxy-CO—, alkyl-S(O)₂—, cycloalkyl-alkyl-S(O)₂—, carbocyclic aryl-alkyl-S(O)₂— or hetero-carbocyclic aryl-alkyl-S(O)₂—;
R₂ or R₃, independently of one another represent alkyl, cycloalkyl-alkyl cycloalkyl being unsubstituted or substituted by alkyl or by carboxy-alkyl, by alkoxy-CO-alkyl or by carbocyclic aryl-alkoxy-CO-alkyl, or represent carbocyclic or heterocyclic aryl-alkyl, alkoxy-CO-alkyl or by carbocyclic aryl-alkoxy-CO-alkyl; or
R₂ and R₃ together represent C₂-C₈-alkylene;
wherein ring A and ring B, independent of one another, or carbocyclic or heterocyclic aryl, is otherwise unsubstituted or substituted by a substituent selected from the group consisting of halogen, NO₂, CN, OH, alkyl, alkoxy-alkyl, halo-alkyl, alkoxy, alkoxy-alkoxy, alkyl-S(O)ₙ, cycloalkyl-alkyl-S(O)ₙ, carbocyclic or heterocyclic aryl-alkyl-S(O)ₙ, n being in each case the integer 0, 1 or 2, halo-alkoxy, carbocyclic or heterocyclic aryl, and alkanoyl(oxy), and wherein two substituents together with the two carbon atoms to which they are attached can form a 5- or 6-membered ring which can be unsubstituted or otherwise substituted by a substitutent selected from the group as specified above.

Preferred is a compound of formula (I′)

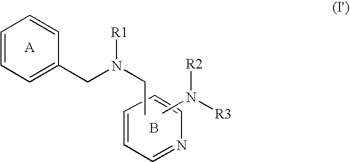

wherein R₁ is carbocyclic or heterocyclic aryl, alkoxy-CO—, cylcoalkyl-alkoxy-CO—, carbocyclic aryl-alkoxy-CO—, alkyl-S(O)₂—, cycloalkyl-alkyl-S(O)₂—, carbocyclic aryl-alkyl-S(O)₂— or hetero-carbocyclic aryl-alkyl-S(O)₂—;
R₂ or R₃, independently of one another represent alkyl, cycloalkyl-alkyl cycloalkyl being unsubstituted or substituted by alkyl or by carboxy-alkyl, by alkoxy-CO-alkyl or by carbocyclic aryl-alkoxy-CO-alkyl, or represent carbocyclic or heterocyclic aryl-alkyl, alkoxy-CO-alkyl or by carbocyclic aryl-alkoxy-CO-alkyl; or
R₂ and R₃ together represent C₂-C₈-alkylene;
wherein ring A and ring B, independent of one another, or carbocyclic or heterocyclic aryl, is otherwise unsubstituted or substituted by a substituent selected from the group consisting of halogen, NO₂, CN, OH, alkyl, alkoxy-alkyl, halo-alkyl, alkoxy, alkoxy-alkoxy, alkyl-S(O)ₙ, cycloalkyl-alkyl-S(O)ₙ, carbocyclic or heterocyclic aryl-alkyl-S(O)ₙ, n being in each case the integer 0, 1 or 2, halo-alkoxy, carbocyclic or heterocyclic aryl, and alkanoyl(oxy), and wherein two substituents together with the two carbon atoms to which they are attached can form a 5- or 6-membered ring which can be unsubstituted or otherwise substituted by a substitutent selected from the group as specified above;
in free form or in salt form.

Preferred is a compound of formula (I A)

wherein R₁ is a heterocyclic ring selected from the group consisting of

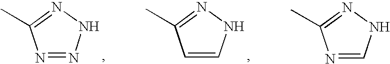

being in each case unsubstituted or N-substituted by a substituent selected from the group consisting of C₁-C₇alkyl, C₃-C₇-cycloalkyl-C₁-C₇alkyl, and phenyl-C₁-C₇-alkyl; or is phenyl, phenacyl, phenyl-S(O)₂ C₂-C₇-alkoxycarbonyl, C₂-C₇-alkoxy-thiocarbonyl, carbamoyl, C₁-C₇-alkyl-alkylamino-carbonyl, di-C₁-C₇-alkyl-alkylamino-carbonyl, or C₁-C₇-alkyl-S(O)₂;
R₂ and R₃, independently of one another, represent C₁-C₇-alkyl, C₃-C₇-cycloalkyl-C₁-C₇-alkyl cycloalkyl being unsubstituted or substituted by a substituent selected from the group consisting of C₁-C₇alkyl, of carboxy-C₁-C₇alkyl, of C₁-C₇alkoxycarbonyl-C₁-C₇-alkyl, of carbamoyl-C₁-C₄-alkyl, of C₁-C₇-alkyl-carbamoyl-C₁-C₄-alkyl, of di-C₁-C₇-alkyl-carbamoyl-C₁-C₄-alkyl, of hydroxyl-C₁-C₄-alkyl, of amino-C₁-C₄-alkyl, or represent phenyl-C₁-C₇-alkyl, naphthyl-C₁-C₇alkyl, pyridyl-C₁-C₇-alkyl, or C₂-C₇-alkoxycarbonyl; or
R₂ and R₃ together represent C₂-C₆-alkylene being unsubstituted or substituted by a substituent selected from the group consisting of C₁-C₇-alkyl, C₃-C₈-cycloalkyl, and heterocyclyl;
R₄, R₅, R₆, R₇, and R₈, independently of one another, represent hydrogen, halogen, NO₂, CN, OH, C₁-C₇-alkyl, phenyl-C₁-C₇-alkyl, naphthyl-C₁-C₇alkyl, pyridyl-C₁-C₇-alkyl, C₃-C₇-cycloalkyl-C₁-C₇-alkyl, C₁-C₇-alkoxy-C₁-C₇-alkyl, phenyl-C₁-C₇-alkoxy, naphthyl-C₁-C₇alkoxy, pyridyl-C₁-C₇-alkoxy, C₃-C₇-cycloalkyl-C₁-C₇alkoxy, halo-C₁-C₇-alkyl, C₁-C₇-alkoxy, C₁-C₇-alkoxy-C₁-C₇-alkoxy, C₁-C₇alkyl-S(O)ₙ—, phenyl-C₁-C₇alkyl-S(O),, naphthyl-C₁-C₇-alkyl-S(O)ₙ, pyridyl-C₁-C₇-alkyl-S(O)ₙ, halo-C₁-C₇alkoxy, phenyl, naphthyl, pyridyl, and C₂-C₇-alkanoyl(oxy);
where, in each case, n is the integer 0, 1 or 2; a phenyl, biphenyl, naphthyl or pyridyl substituent is, independently of one another is unsubstituted or substituted by a substitutent selected from the group consisting of the substituents specified under variables R₄, R₅, R₆, and R₇; or a pharmaceutically acceptable salt thereof.

Preferred is a compound of formula (I A) wherein

R₁ is a heterocyclic ring selected from the group consisting of

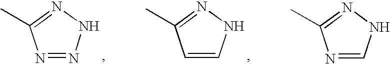

being in each case N-substituted by C₁-C₇-alkyl, C₃-C₇-cycloalkyl-C₁-C₇-alkyl, and phenyl-C₁-C₇alkyl; or
R₁ is phenyl, formyl, phenacyl, phenyl-S(O)₂, carboxy, C₂-C₇-alkoxycarbonyl, carbamoyl, C₁-C₇-alkyl-alkylamino-carbonyl, di-C₁-C₇-alkyl-alkylamino-carbonyl, or C₁-C₇-alkyl-S(O)₂;
R₂ and R₃, independently of one another, represents phenyl, pyridyl, C₁-C₇-alkyl, C₁-C₇-alkanoyl, C₁-C₇alkyl which is substituted by C₃-C₇-cycloalkyl, whereby C₃-C₇-cycloalkyl itself is unsubstituted or substituted by C₁-C₇-alkyl (which itself is unsubstituted or substituted by hydroxyl, amino, carboxy, C₁-C₇alkoxy-carbonyl, carbamoyl, or carbamoyl which is mono- or di-substituted by C₁-C₇alkyl), or represents C₃-C₇-cycloalkyl which is unsubstituted or substituted by C₁-C₇alkyl, C₃-C₇cycloalkyl which is interrupted by 0 and which is unsubstituted or substituted by C₁-C₇-alkyl, or C₃-C₇cycloalkyl which is interrupted by NH which is unsubstituted or N-substituted by C₁-C₇-alkyl, hydroxy-C₁-C₇-alkyl or amino-C₁-C₇ alkyl;
R₂ and R₃ together represent C₂-C₇-alkylene which is unsubstituted or substituted by C₁-C₇-alkyl, C₁-C₇-alkyl which is substituted by C₁-C₇alkyl, C₁-C₇alkoxy-C₁-C₇-alkyl carboxy, C₁-C₇-alkoxy-carbonyl, C₃-C₇cycloalkyl or by phenyl, or represent C₂-C₇alkylene which is interrupted by O or N—C₁-C₇alkyl; or represent C₂-C₇-alkylene to which a C₃-C₇-cycloalkyl is either annelated or attached to in spiro form; and
R₄, R₅, R₆, R₇, and R₅, independently of one another, represent hydrogen, halogen, NO₂, CN, halo-C₁-C₇alkyl, phenyl or pyridyl;
or a pharmaceutically acceptable salt thereof.

Especially preferred is a compound of formula (I B)

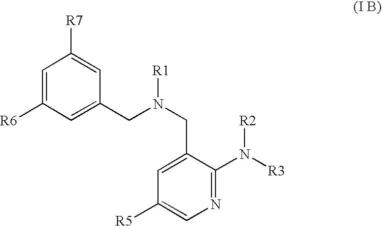

wherein R₁ is a heterocyclic ring selected from the group consisting of

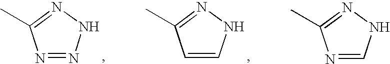

being in each case unsubstituted or N-substituted by C₁-C₇alkyl;
or is C₂-C₇-alkoxycarbonyl or C₁-C₇alkyl-S(O)₂;
R₂ is C₁-C₇-alkyl;
R₃ is C₃-C₇-cycloalkyl-C₁-C₇-alkyl cycloalkyl being unsubstituted or substituted by a substituent selected from the group consisting of C₁-C₇alkyl and of carboxycarbonyl-C₁-C₇ alkyl; or
R₄ is halo-C₁-C₇-alkyl, especially trifluoromethyl;
R₅ is hydrogen;
R₆ is halo-C₁-C₇-alkyl, especially trifluoromethyl; and
R₇ is halogen, NO₂, CN, or halo-C₁-C₇-alkyl, especially trifluoromethyl;
or a pharmaceutically acceptable salt thereof.

The invention relates in particular to the novel compounds shown in the examples and to the modes of preparation described therein.

The invention relates to processes for the preparation of the compounds according to the invention. The preparation of compounds of formula (I) or a salt thereof is carried out in a manner known per se and comprises, for example, as described in the following general schemes:

General synthesis of compounds of formula (I), especially exemplified for compounds of formulae (I A) and (I B), is outlined in the following Schemes:

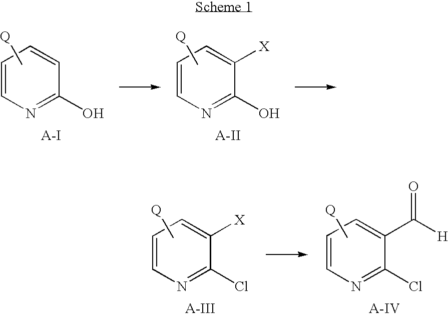

Required starting compounds can be synthesized according to scheme 1. Starting from pyridone (A-I), halogenation with an appropriate reagent such as N-bromosuccinimide and bromine at −20-30° C. in inert solvents such as dichloromethane gives compound A-II. Treatment with an appropriate reagent such as phosphoryl chloride at −20˜30° C. affords compound A-III. Halogen-metal exchange can be performed with alkyl metal reagents such as n-butyl lithium, and formylation with a formylating agent such as N,N-dimethylformamide gives compound A-IV.

The compounds in this invention and depicted as compound VII can be prepared according to the following schemes 2-5.

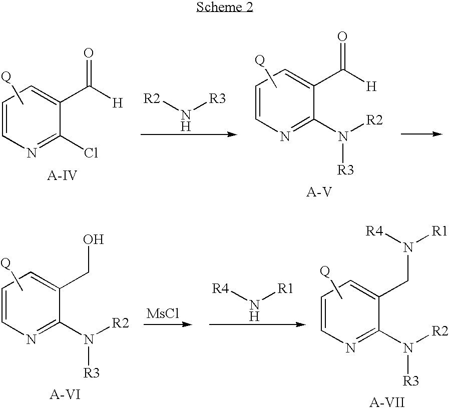

Compound A-V is prepared by amination of compound A-IV in the presence of an appropriate base such as diisopropylethylamine, potassium carbonate, triethylamine, or sodium hydride. Reduction of the aldehyde group by using a reducing reagent such as sodium borohydride or lithium aluminum hydride gives the corresponding alcohol (A-VI). After conversion of the alcohol to a leaving group, for example, conversion to methanesulfonate, chloride or bromide, a secondary amine is alkylated in the presence of a base such as diisopropylethylamine, triethylamine or potassium carbonate to give a desired product.

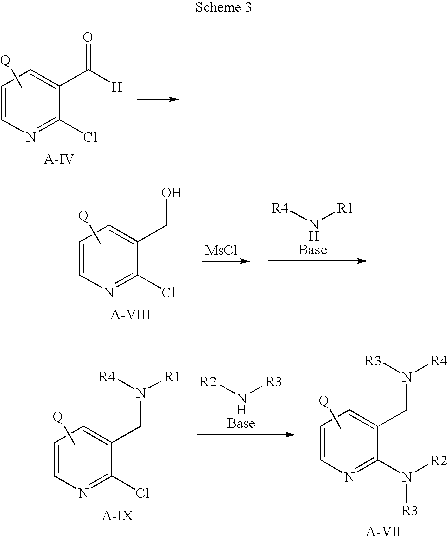

Alternatively, compound A-VII can be synthesized from compound A-IV according to scheme 3 by utilizing similar conditions to that in scheme 2.

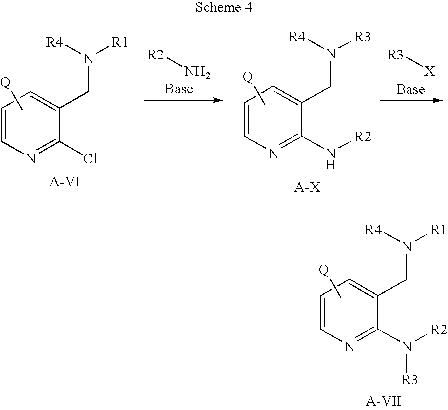

Synthesis of compound A-VII can also be performed from compounds A-VI as shown in scheme 4. A primary amine is reacted with compounds A-VI in the presence of a base such as diisopropylethylamine, sodium hydride, triethylamine or potassium bis(trimethylsilyl)amide. The resulting secondary amine can be reacted with an alkylating reagent such as alkyl bromide, alkyl iodide and alkyl methansulfonate, or a acid chloride, with a base such as diisopropylethylamine, sodium hydride, triethylamine or potassium bis(trimethylsilyl)amide to give a desired compound.

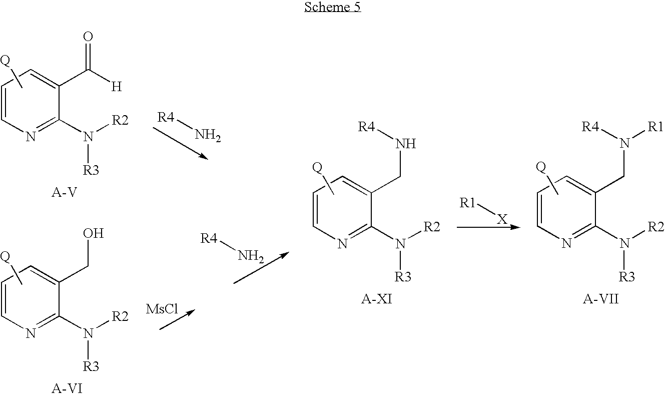

Compound A-VII can also be prepared according to scheme 5. Compound A-XI can be obtained by reductive amination from compound A-V by use of a reagent such as sodium borohydride or sodium triacetoxyborohydride, or amination after conversion of the alcohol to a leaving group such as chloride, bromide or methansulfonate in the presence of a base such as diisopropylethylamine, triethylamine or potassium carbonate. The resulting compound A-XI can be reacted with an appropriate reagent such as acid chlorides, chloroformates, alkyl halides in the presence, or in the absence, of a base such as potassium carbonate, sodium carbonate, triethylamine or diisopropylethylamine to give a desired compound A-VII.

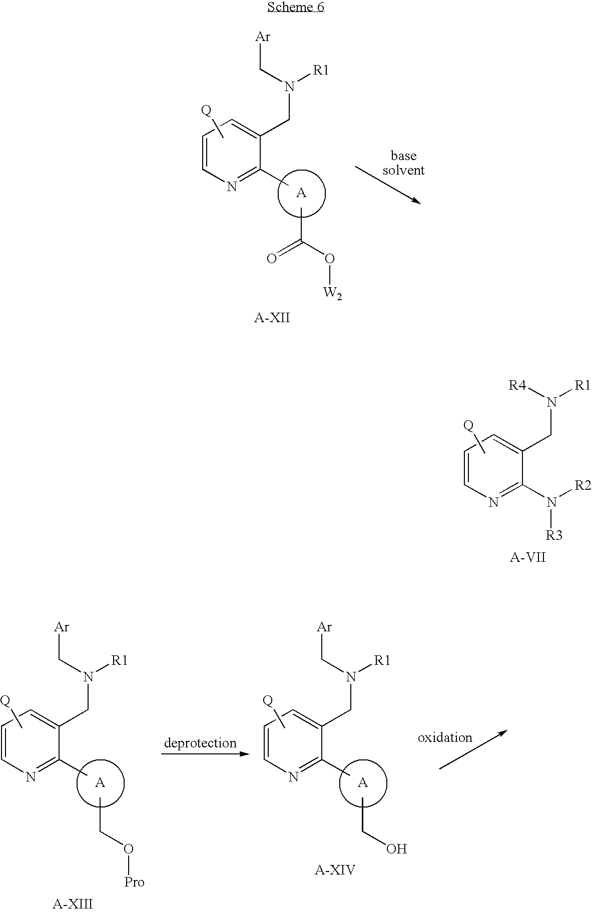

Compound A-XII, wherein A is linker, W2 is C1-C6 alkyl and Pro is a protective group, and A-XIII can also be prepared according to scheme 2-5. Compound A-VII can be obtained by hydrolysis of compound A-XII by use of a base such as sodium hydroxide, or lithium hydroxide aqueous solution in an appropriate solvent, such as methanol, ethanol or THF. Compound A-XIV can be obtained by deprotection of A-XIII by use of appropriate reagent. Compound A-XIV can be reacted with an appropriate oxidative reagent such as Pyridinium Chlorochromate, Pyridinium dichromate, Dess-Martin periodinane, swern-oxidation, NaClO₂, and TEMPO-oxidation to give a desired compound A-VII.

Secondary amines (HNR₂R₃) can be prepared from amines and aldehydes by reductive amination with an appropriate reagent such as sodium borohydride or sodium triacetoxyborohyde, or from amines and alkyl halides by alkylation in the presence of a base such as sodium carbonate, potassium carbonate, triethylamine or diisopropylethylamine.

In view of the close relationship between the novel compound in the free form and in the form of its salts, in the preceding text and below the free compound or its salts may correspondingly and advantageously also be understood as meaning the corresponding salts or the free compound.

The novel compounds including their salts of salt-forming compounds can also be obtained in the form of their hydrates or can include other solvents used for crystallization.

Depending on the choice of the starting materials and procedures, the novel compounds can be present in the form of one of the possible isomers or as mixtures thereof, for example as pure optical isomers, such as antipodes, or as isomer mixtures, such as racemates, diastereoisomer mixtures or racemate mixtures, depending on the number of asymmetric carbon atoms.

Racemates and diastereomer mixtures obtained can be separated into the pure isomers or racemates in a known manner on the basis of the physicochemical differences of the components, for example by fractional crystallization. Racemates obtained may furthermore be resolved into the optical antipodes by known methods, for example by recrystallization from an optically active solvent, chromatography on chiral adsorbents, with the aid of suitable microorganisms, by cleavage with specific immobilized enzymes, via the formation of inclusion compounds, for example using chiral crown ethers, only one enantiomer being complexed, or by conversion into diastereomeric salts, for example by reaction of a basic final substance racemate with an optically active acid, such as a carboxylic acid, for example tartaric or malic acid, or sulfonic acid, for example camphorsulfonic acid, and separation of the diastereomer mixture obtained in this manner, for example on the basis of its differing solubilities, into the diastereomers from which the desired enantiomer can be liberated by the action of suitable agents. The more active enantiomer is advantageously isolated.

The invention also relates to those embodiments of the process, according to which a compound obtainable as an intermediate in any step of the process is used as a starting material and the missing steps are carried out or a starting material in the form of a derivative or salt and/or its racemates or antipodes is used or, in particular, formed under the reaction conditions.

In the process of the present invention, those starting materials are preferably used which lead to the compounds described as particularly useful at the beginning. The invention likewise relates to novel starting materials which have been specifically developed for the preparation of the compounds according to the invention, to their use and to processes for their preparation.

The invention likewise relates to a combination of a compound of formula (I), (I′), (I A) or (I B), respectively, or a pharmaceutically acceptable salt thereof with a further active principle.

The combination may be made for example with the following active principles, selected from the group consisting of a:

(i) HMG-Co-A reductase inhibitor or a pharmaceutically acceptable salt thereof,
(ii) angiotensin II receptor antagonist or a pharmaceutically acceptable salt thereof,
(iii) angiotensin converting enzyme (ACE) Inhibitor or a pharmaceutically acceptable salt thereof,
(iv) calcium channel blocker or a pharmaceutically acceptable salt thereof,
(v) aldosterone synthase inhibitor or a pharmaceutically acceptable salt thereof,
(vi) aldosterone antagonist or a pharmaceutically acceptable salt thereof,
(vii) dual angiotensin converting enzyme/neutral endopeptidase (ACE/NEP) inhibitor or a pharmaceutically acceptable salt thereof,
(viii) endothelin antagonist or a pharmaceutically acceptable salt thereof,
(ix) renin inhibitor or a pharmaceutically acceptable salt thereof,
(x) diuretic or a pharmaceutically acceptable salt thereof, and
(xi) an ApoA-I mimic.

An angiotensin II receptor antagonist or a pharmaceutically acceptable salt thereof is understood to be an active ingredients which bind to the AT₁-receptor subtype of angiotensin II receptor but do not result in activation of the receptor. As a consequence of the inhibition of the AT₁ receptor, these antagonists can, for example, be employed as antihypertensives or for treating congestive heart failure.

The class of AT₁ receptor antagonists comprises compounds having differing structural features, essentially preferred are the non-peptidic ones. For example, mention may be made of the compounds which are selected from the group consisting of valsartan, losartan, candesartan, eprosartan, irbesartan, saprisartan, tasosartan, telmisartan, the compound with the designation E-1477 of the following formula

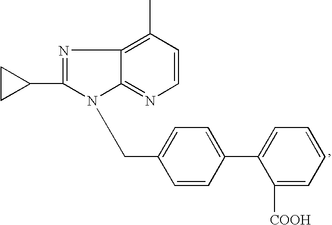

the compound with the designation SC-52458 of the following formula

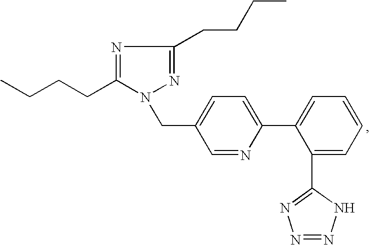

and the compound with the designation ZD-8731 of the following formula

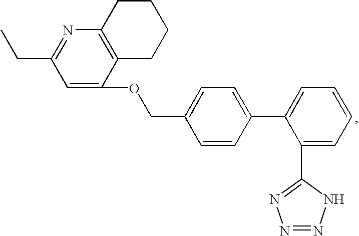

or, in each case, a pharmaceutically acceptable salt thereof.

Preferred AT₁-receptor antagonist are those agents which have been marketed, most preferred is valsartan or a pharmaceutically acceptable salt thereof.

HMG-Co-A reductase inhibitors (also called β-hydroxy-β-methylglutaryl-co-enzyme-A reductase inhibitors) are understood to be those active agents that may be used to lower the lipid levels including cholesterol in blood.

The class of HMG-Co-A reductase inhibitors comprises compounds having differing structural features. For example, mention may be made of the compounds that are selected from the group consisting of atorvastatin, cerivastatin, compactin, dalvastatin, dihydrocompactin, fluindostatin, fluvastatin, lovastatin, pitavastatin, mevastatin, pravastatin, rivastatin, simvastatin, and velostatin, or, in each case, a pharmaceutically acceptable salt thereof.

Preferred HMG-Co-A reductase inhibitors are those agents which have been marketed, most preferred is fluvastatin and pitavastatin or, in each case, a pharmaceutically acceptable salt thereof.

The interruption of the enzymatic degradation of angiotensin I to angiotensin II with so-called ACE-inhibitors (also called angiotensin converting enzyme inhibitors) is a successful variant for the regulation of blood pressure and thus also makes available a therapeutic method for the treatment of congestive heart failure.

The class of ACE inhibitors comprises compounds having differing structural features. For example, mention may be made of the compounds which are selected from the group consisting alacepril, benazepril, benazeprilat, captopril, ceronapril, cilazapril, delapril, enalapril, enaprilat, fosinopril, imidapril, lisinopril, moveltopril, perindopril, quinapril, ramipril, spirapril, temocapril, and trandolapril, or, in each case, a pharmaceutically acceptable salt thereof.

Preferred ACE inhibitors are those agents that have been marketed, most preferred are benazepril and enalapril.

The class of CCBs essentially comprises dihydropyridines (DHPs) and non-DHPs such as diltiazem-type and verapamil-type CCBs.

A CCB useful in said combination is preferably a DHP representative selected from the group consisting of amlodipine, felodipine, ryosidine, isradipine, lacidipine, nicardipine, nifedipine, niguldipine, niludipine, nimodipine, nisoldipine, nitrendipine, and nivaldipine, and is preferably a non-DHP representative selected from the group consisting of flunarizine, prenylamine, diltiazem, fendiline, gallopamil, mibefradil, anipamil, tiapamil and verapamil, and in each case, a pharmaceutically acceptable salt thereof. All these CCBs are therapeutically used, e.g. as anti-hypertensive, anti-angina pectoris or anti-arrhythmic drugs. Preferred CCBs comprise amlodipine, diltiazem, isradipine, nicardipine, nifedipine, nimodipine, nisoldipine, nitrendipine, and verapamil, or, e.g. dependent on the specific CCB, a pharmaceutically acceptable salt thereof. Especially preferred as DHP is amlodipine or a pharmaceutically acceptable salt, especially the besylate, thereof. An especially preferred representative of non-DHPs is verapamil or a pharmaceutically acceptable salt, especially the hydrochloride, thereof.

Aldosterone synthase inhibitor is an enzyme that converts corticosterone to aldosterone to by hydroxylating cortocosterone to form 18-OH-corticosterone and 18-OH-corticosterone to aldosterone. The class of aldosterone synthase inhibitors is known to be applied for the treatment of hypertension and primary aldosteronism comprises both steroidal and non-steroidal aldosterone synthase inhibitors, the later being most preferred.

Preference is given to commercially available aldosterone synthase inhibitors or those aldosterone synthase inhibitors that have been approved by the health authorities.

The class of aldosterone synthase inhibitors comprises compounds having differing structural features. For example, mention may be made of the compounds which are selected from the group consisting of the non-steroidal aromatase inhibitors anastrozole, fadrozole (including the (+)-enantiomer thereof), as well as the steroidal aromatase inhibitor exemestane, or, in each case where applicable, a pharmaceutically acceptable salt thereof.

The most preferred non-steroidal aldosterone synthase inhibitor is the (+)-enantiomer of the hydrochloride of fadrozole (U.S. Pat. Nos. 4,617,307 and 4,889,861) of formula

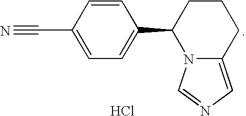

A preferred steroidal aldosterone antagonist is eplerenone of the formula

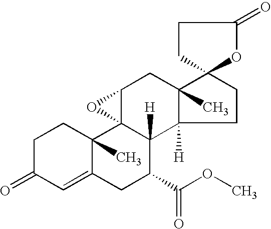

or
spironolactone.

A preferred dual angiotensin converting enzyme/neutral endopetidase (ACE/NEP) inhibitor is, for example, omapatrilate (cf. EP 629627), fasidotril or fasidotrilate, or, if appropriable, a pharmaceutically acceptable salt thereof.

A preferred endothelin antagonist is, for example, bosentan (cf. EP 526708 A), furthermore, tezosentan (cf. WO 96/19459), or in each case, a pharmaceutically acceptable salt thereof.

A renin inhibitor is, for example, a non-peptidic renin inhibitor such as the compound of formula

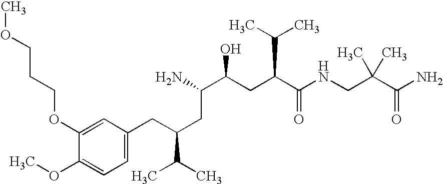

chemically defined as 2(S),4(S),5(S),7(S)—N-(3-amino-2,2-dimethyl-3-oxopropyl)-2,7-di(1-methylethyl)-4-hydroxy-5-amino-8-[4-methoxy-3-(3-methoxy-propoxy)phenyl]-octanamide. This representative is specifically disclosed in EP 678503 A. Especially preferred is the hemi-fumarate salt thereof.

A diuretic is, for example, a thiazide derivative selected from the group consisting of chlorothiazide, hydrochlorothiazide, methylclothiazide, and chlorothalidon. The most preferred is hydrochlorothiazide.

An ApoA-I mimic is, for example, D4F peptide, especially of formula D-W-F-K-A-F-Y-D-K-V-A-E-K-F-K-E-A-F

Preferably, the jointly therapeutically effective amounts of the active agents according to the combination of the present invention can be administered simultaneously or sequentially in any order, separately or in a fixed combination.

The structure of the active agents identified by generic or tradenames may be taken from the actual edition of the standard compendium “The Merck Index” or from databases, e.g. IMS LifeCycle (e.g. IMS World Publications). The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo. The invention in particular relates to a compound of formula (I), (I′), (I A) or (I B), respectively, or a pharmaceutically acceptable salt thereof, for the treatment of the human or animal body.

The invention likewise relates to the use of the compounds of the formula I or of pharmaceutically acceptable salts of compounds of this type with salt-forming properties, in particular as pharmacological, primarily CETP inhibitors, active substances. In this connection, they can be used, preferably in the form of pharmaceutically acceptable preparations, in a method for the prophylactic and/or therapeutic treatment of the animal or human body, in particular as inhibitors of CETP.

The invention in particular relates to the use of a compound of formula (I), (I′), (I A) or (I B), respectively, or a pharmaceutically acceptable salt thereof, optionally in combination with at least one composition for the treatment of cardiovascular diseases and related conditions and diseases listed hereinbefore or hereinafter, for the manufacture of a medicament for the prophylaxis or treatment of or delay progression to overt to diseases in which CETP is involved (e.g., hyperlipidemia, arteriosclerosis, atherosclerosis, peripheral vascular disease, dyslipidemia, hyperbetalipoproteinemia, hypoalphalipoproteinemia, hypercholesterolemia, hypertriglyceridemia, familial hypercholesterolemia, cardiovascular disorder, coronary heart disease, coronary artery disease, coronary vascular disease, angina, ischemia, heart ischemia, thrombosis, cardiac infarction such as myocardial infarction, stroke, peripheral vascular disease, reperfusion injury, angioplasty restenosis, hypertension, congestive heart failure, diabetes such as type II diabetes mellitus, diabetic vascular complications, obesity or endotoxemia etc.), particularly as prophylactic or therapeutic agents for hyperlipidemia or arteriosclerotic diseases and also for the treatment of infection (or egg embryonation) of schistosoma.

The present invention likewise relates to a method for the prophylaxis or treatment of or delay progression to overt to diseases in which CETP is involved (e.g., hyperlipidemia, arteriosclerosis, atherosclerosis, peripheral vascular disease, dyslipidemia, hyperbetalipoproteinemia, hypoalphalipoproteinemia, hypercholesterolemia, hypertriglyceridemia, familial hypercholesterolemia, cardiovascular disorder, coronary heart disease, coronary artery disease, coronary vascular disease, angina, ischemia, heart ischemia, thrombosis, cardiac infarction such as myocardial infarction, stroke, peripheral vascular disease, reperfusion injury, angioplasty restenosis, hypertension, congestive heart failure, diabetes such as type II diabetes mellitus, diabetic vascular complications, obesity or endotoxemia etc.), particularly as prophylactic or therapeutic agents for hyperlipidemia or arteriosclerotic diseases, comprising administering to an animal, including man, in need thereof, a formula (I), (I′), (I A) or (I B), respectively, or a pharmaceutically acceptable salt thereof, optionally in combination with at least one composition for the treatment of cardiovascular diseases and related conditions and diseases listed hereinbefore or hereinafter.

The present invention likewise relates to a pharmaceutical composition comprising a formula (I), (I′), (I A) or (I B), respectively, or a pharmaceutically acceptable salt thereof, optionally in combination with at least one composition for the treatment of cardiovascular diseases and related conditions and diseases listed hereinbefore or hereinafter, for the prophylaxis or treatment of or delay progression to overt to diseases in which CETP is involved (e.g., hyperlipidemia, arteriosclerosis, atherosclerosis, peripheral vascular disease, dyslipidemia, hyperbetalipoproteinemia, hypoalphalipoproteinemia, hypercholesterolemia, hypertriglyceridemia, familial hypercholesterolemia, cardiovascular disorder, coronary heart disease, coronary artery disease, coronary vascular disease, angina, ischemia, heart ischemia, thrombosis, cardiac infarction such as myocardial infarction, stroke, peripheral vascular disease, reperfusion injury, angioplasty restenosis, hypertension, congestive heart failure, diabetes such as type II diabetes mellitus, diabetic vascular complications, obesity or endotoxemia etc.), particularly as prophylactic or therapeutic agents for hyperlipidemia or arteriosclerotic diseases.

The pharmaceutical preparations according to the invention which contain the compound according to the invention or pharmaceutically acceptable salts thereof are those for enteral, such as oral, furthermore rectal, and parenteral administration to (a) warm-blooded animal(s), the pharmacological active ingredient being present on its own or together with a pharmaceutically acceptable carrier. The daily dose of the active ingredient depends on the age and the individual condition and also on the manner of administration.

The dose of the active ingredient depends on the warm-blooded animal species, the age and the individual condition and on the manner of administration.

The following examples illustrate the invention described above; however, they are not intended to limit its extent in any manner. Temperatures are indicated in degrees Celsius.

### EXAMPLES

### Abbreviations

AcOEt: ethyl acetate, AcOH: acetic acid, BuLi: butyl lithium, DEAD: diethyl azadicarboxylate, DHP: dihydropyrane, DMAP: 4-(N,N-dimethylamino)pyridine, DMF: N,N-dimethylformamide, EtOH: ethanol, Hex: n-hexane, iPr: isopropyl, IPA: isopropylalcohol, KOt-Bu: potassium tert-butoxide, LiAlH₄: lithium aluminum hydride, MeOH: methanol, NaBH₄: sodium tetraborohydride, NBS: N-bromosuccinimide, Pd(Ph₄)₄: tetrakis(triphenylphosphine)palladium(0), PCC: Pyridinium chlorochromate, POCl₃: phosphorus(III)oxychloride, PPh₃: triphenylphosphine, PS-DIEA: Polymer-supported diisopropylethylamine, sat.: saturated, SOCl₂: thionyl chloride, TEA: triethylamine, TFA: trifluoroacetic acid, THF: tetrahydrofuran.

### Example 1

### Synthesis of [3-({[3,5-bis(trifluoromethyl)benzyl](2-methyl-2H-tetrazol-5-yl)amino}methyl)5-trifluoromethylpyridin-2-yl](cyclopentylmethyl)ethylamine

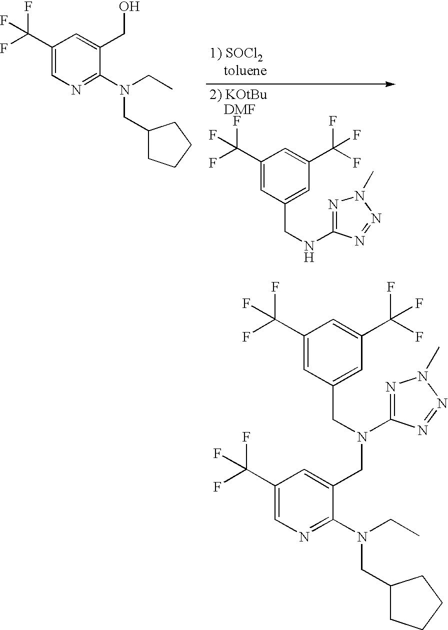

A mixture of [2-(cyclopentylmethylethylamino)-5-(trifluoromethyl)pyridin-3-yl]methanol (55 mg, 0.18 mmol) and thionyl chloride (17 μL, 0.22 mmol) in toluene (0.50 mL) is stirred at ambient temperature for 3 hours. The mixture is concentrated in vacuo. After [3,5-bis(trifluoromethyl)phenylmethyl](2-methyl-2H-tetrazol-5-yl)amine (89 mg, 0.27 mmol) and DMF (0.5 mL) are added to the mixture, the mixture is stirred and then potassium t-butoxide (31 mg, 0.29 mmol) is added and the mixture is further stirred for 20 min. After adding sat. ammonium chloride, the mixture is extracted with ethyl acetate. The combined organic layer is washed with brine, dried over magnesium sulfate, filtrated, and concentrated. The resulting mixture is purified by silica gel column chromatography (ethyl acetate/hexane=5/95 to 35/65) to give [3-({[3,5-bis(trifluoromethyl)benzyl](2-methyl-2H-tetrazol-5-yl)amino}methyl)-5-(trifluoromethyl)pyridin-2-yl](cyclopentylmethyl)ethylamine (39 mg, 36% yield).

¹H-NMR (400 MHz, CDCl₃), δ (ppm): 0.94-1.05 (m, 2H), 1.04 (t, 3H), 1.41-1.58 (m, 6H), 2.00-2.09 (m, 1H), 3.15 (d, 2H), 3.17 (q, 2H), 3.82 (s, 3H), 4.46 (s, 2H), 4.55 (s, 2H), 7.69-7.70 (m, 2H), 7.80-7.82 (m, 2H), 8.47-8.79 (m, 1H).

ESI-MS m/z: 610 [M+1]⁺.

### Example 2

The following compounds are prepared from {2-[(cyclopentylmethyl)ethylamino]-5-(trifluoromethyl)pyridin-3-yl}methanol and corresponding amines following the procedure of example 1.

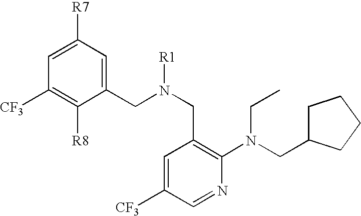

| | | | | MS or | |
|---|---|---|---|---|---|
| | | | | Rf | ¹H-NMR (400 MHz), δ (ppm) or HPLC/UPLC |
| No. | R₇ | R₈ | R₁ | value | Retention time |
| 2-1 | Cl | H | 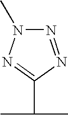 | 576[M + 1]⁺ | CDCl₃: 1.01-1.05 (m, 2H), 1.04 (t, 3H), 1.41-1.60 (m, 6H), 2.04-2.13 (m, 1H), 3.16 (d, 2H),3.17 (q, 2H), 4.23 (s, 3H), 4.53 (s, 2H), 4.65 (s,2H), 7.28-7.30 (m, 1H), 7.32-7.35 (m, 1H), 7.47-7.50 (m, 1H), 7.51-7.53 (m, 1H), 8.39-8.42 (m,1H). |
| 2-2 | NO2 | H | 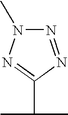 | 587[M + 1]⁺ | CDCl₃: 1.00-1.08 (m, 2H), 1.06 (t, 3H), 1.41-1.62 (m, 6H), 2.04- 2.12 (m, 1H), 3.17-3.20 (m,4H), 4.23 (s, 3H), 4.64 (s, 2H), 4.72 (s, 2H),7.50-7.53 (m, 1H), 7.72- 7.74 (m, 1H), 8.21-8.24 (m, 1H), 8.35-8.37 (m, 1H), 8.39-8.41 (m,1H). |
| 2-3 | CN | H | 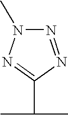 | 567[M + 1]⁺ | CDCl₃: 1.00-1.09 (m, 2H), 1.07 (t, 3H), 1.42-1.62 (m, 6H), 2.04- 2.15 (m, 1H), 3.16-3.21 (m,4H), 4.23 (s, 3H), 4.57 (s, 2H), 4.68 (s, 2H),7.49-7.52 (m, 1H), 7.60-7.61 (m, 1H), 7.66-7.67 (m, 1H), 7.78-7.79 (m, 1H), 8.40-8.42 (m,1H). |
| 2-4 | CF3 | H | 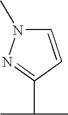 | 608[M + 1]⁺ | CDCl₃: 0.97-1.09 (m, 2H), 1.04 (t, 3H), 1.40-1.60 (m, 6H), 2.00-2.10 (m, 1H), 3.14-3.19 (m,4H), 3.76 (s, 3H), 4.40 (s, 2H), 4.48 (s, 2H),5.39-5.42 (m, 1H), 7.14-7.16 (m, 1H), 7.25-7.26 (m, 1H), 7.66-7.68 (m, 1H), 7.71-7.74 (m,1H), 8.37-8.39 (m, 1H). |
| 2-5 | Cl | H | 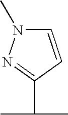 | 574[M + 1]⁺ | CDCl₃: 1.02-1.08 (m, 2H), 1.04 (t, 3H), 1.40-1.60 (m, 6H), 2.01-2.11 (m, 1H), 3.13-3.19(m, 4H), 3.76 (s, 3H), 4.38-4.41 (m, 4H), 5.40(d, 1H), 7.15 (d, 1H), 7.34-7.36 (m, 1H), 7.38-7.40 (m, 1H), 7.44-7.46 (m, 1H), 7.72-7.74(m, 1H), 8.36-8.39 (m, 1H). |
| 2-6 | Cl | H | 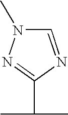 | 575[M + 1]⁺ | CDCl₃: 1.00-1.07 (m, 2H), 1.03 (t, 3H), 1.40-1.60 (m, 6H), 2.02-2.12 (m, 1H), 3.13-3.19(m, 4H), 3.80 (s, 3H), 4.48 (s, 2H), 4.59 (s, 2H),7.30-7.32 (m, 1H), 7.34-7.36 (m, 1H), 7.44-7.46 (m, 1H), 7.60-7.62 (m, 1H), 7.74-7.75(m, 1H), 8.37-8.39 (m, 1H). |
| 2-7 | CF3 | H | 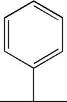 | 604[M + 1]⁺ | CDCl₃: 1.03-1.12 (m, 2H), 1.09 (t, 3H), 1.42-1.65 (m, 6H), 2.04-2.14 (m, 1H), 3.15-3.22(m, 4H), 4.49 (s, 2H), 4.67 (s, 2H), 6.64- 6.67(m, 2H), 6.80-6.84 (m, 1H), 7.18-7.24 (m,2H), 7.64-7.68 (m, 3H), 7.77-7.79 (m, 1H),8.42-8.44 (m, 1H). |
| 2-9 | Cl | H | 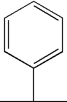 | 570[M + 1]⁺ | CDCl₃: 1.03-1.12 (m, 2H), 1.10 (t, 3H), 1.44-1.66 (m, 6H), 2.05-2.15 (m, 1H), 3.16-3.23(m, 4H), 4.48 (s, 2H), 4.60 (s, 2H), 6.61-6.66(m, 2H), 6.77-6.83 (m, 1H), 7.18-7.23 (m,2H), 7.36-7.41 (m, 1H), 7.50-7.52 (m, 1H),7.63-7.67 (m, 1H), 8.42-8.45 (m, 1H). |

### Example 3

### Synthesis of [3,5-bis(trifluoromethyl)benzyl]{2-[(cyclopentylmethyl)ethylamino]-5-(trifluoromethyl)pyridin-3-ylmethyl}carbamic acid methyl ester

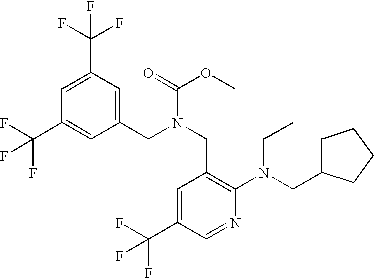

To a stirred solution of (3-{[3,5-bis(trifluoromethyl)benzylamino]methyl}-5-(trifluoromethyl)pyridin-2-yl}(cyclopentylmethyl)ethylamine (150 mg, 0.25 mmol), triethylamine (37 mg, 0.37 mmol) and N,N-dimethylaminopyridine (3 mg, 0.025 mmol) in THF (2.5 mL), methyl chloroformate (23 μL, 0.30 mmol) is added at room temperature. The mixture is stirred for 20 h and then water and sat. sodium bicarbonate aqueous solution are added. The mixture is extracted with dichloromethane and the organic layer is washed with water and brine, dried over sodium sulfate and concentrated in vacuo. The residue is purified with reverse-phase preparative HPLC (0.1% TFA-H₂O to CH₃CN) to give 72 mg of [3,5-bis(trifluoromethyl)benzyl]{2-[(cyclopentylmethyl)ethylamino]-5-(trifluoromethyl)pyridin-3-ylmethyl}carbamic acid methyl ester (50%) as pale yellow oil. ¹H-NMR (400 MHz) a (ppm), CDCl3: 0.90-1.08 (m, 2H), 1.03 (t, 3H), 1.50-1.60 (m, 6H), 2.04-2.12 (m, 1H), 3.10-3.15 (m, 4H), 3.87 (s, 3H), 4.31-4.54 (m, 4H), 7.44-7.65 (m, 3H), 7.76-7.78 (m, 1H), 8.40-8.42 (m, 1H). ESI-MS: 586 [M+1]⁺

### Example 4

The following compounds are prepared from (3-{[3,5-bis(trifluoromethyl)benzylamino]methyl)-5-(trifluoromethyl)pyridin-2-yl}(cyclopentylmethyl)ethylamine following the procedure of example 3 by use of appropriate reagents and conditions.

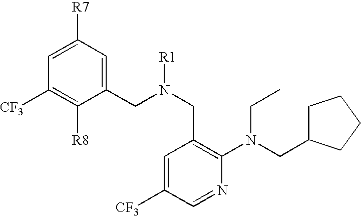

| No. | R₇ | R₈ | R₁ | MS | ¹H-NMR (400 MHz), δ (ppm) |
|---|---|---|---|---|---|
| 4-1 | CF₃ | H | 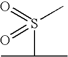 | 606[M + 1]⁺ | CDCl₃: 1.02-1.08 (m, 2H), 1.05 (t, 3H), 1.47-1.64 (m, 6H), 2.04-2.14 (m, 1H), 3.03 (s, 3H),3.11-3.18 (m, 4H), 4.33 (s, 2H), 4.46 (s, 2H),7.50-7.52 (m, 2H), 7.65-7.67 (m, 1H), 7.70-7.72 (m, 1H), 8.28-8.30 (m, 1H). |
| 4-2 | CF₃ | H | 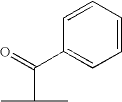 | 632[M + 1]⁺ | CDCl₃: 0.87-1.01 (m, 5H), 1.35-1.49 (m, 6H),1.90-2.10 (m, 1H), 2.90-3.18 (m, 4H), 4.30-4.50 (m, 2H), 4.65-4.82 (m, 2H), 7.39-7.50(m, 6H), 7.60-7.78 (m, 2H), 7.79-7.81 (m,1H), 8.44-8.46 (m, 1H). |
| 4-3 | CF₃ | H | 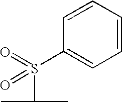 | 668[M + 1]⁺ | CDCl₃: 0.93-1.02 (m, 2H), 0.97 (t, 3H), 1.40-1.53 (m, 6H), 1.96-2.07 (m, 1H), 3.03-3.10(m, 4H), 4.28 (s, 2H), 4.41 (s, 2H), 7.37-7.40(m, 2H), 7.50-7.52 (m, 1H), 7.58-7.64 (m,2H), 7.66-7.72 (m, 2H), 7.90-7.93 (m, 2H),8.25-8.27 (m, 1H). |

### Example 5

The following compounds are prepared from [trans-4-({ethyl[3-hydroxymethyl-5-(trifluoromethyl)pyridin-2-yl]amino}methyl)cyclohexyl]acetic acid ethyl ester and corresponding amines following the procedure of example 1.

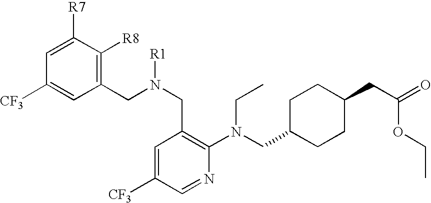

| No. | R₇ | R₈ | R₁ | MS | ¹H-NMR (400 MHz, CDCl₃), δ (ppm) |
|---|---|---|---|---|---|
| 5-1 | CF₃ | H | 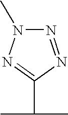 | 710[M + 1]⁺ | 0.82-0.91 (m, 4H), 1.05 (t, 3H), 1.24 (t, 3H),1.48-1.53 (m, 1H), 1.61-1.75 (m, 5H), 2.13 (d,2H), 3.10 (d, 2H), 3.15 (q, 2H), 4.12 (q, 2H),4.22 (s, 3H), 4.61 (s, 2H), 4.68 (s, 2H), 7.49 (d,2H), 7.59 (s, 2H), 7.75 (s, 1H), 8.39 (d, 1H). |
| 5-2 | NO₂ | H | 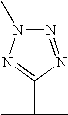 | 687[M + 1]⁺ | 0.82-0.96 (m, 4H), 1.06 (t, 3H), 1.24 (t, 3H),1.62-1.72 (m, 6H), 2.13 (d, 2H), 3.11 (d, 2H),3.17 (q, 2H), 3.49 (d, 2H), 4.11 (q, 2H), 4.22 (s,3H), 4.63 (s, 2H), 4.72 (s, 2H), 7.49 (d, 1H),7.71 (s, 1H), 8.20 (s, 1H), 8.36 (s, 1H), 8.38 (d,1H). |
| 5-3 | CN | H | 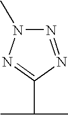 | 667[M + 1]⁺ | 0.80-0.95 (m, 4H), 1.06 (t, 3H), 1.23 (t, 3H),1.63-1.75 (m, 6H), 2.14 (d, 2H), 3.11 (d, 2H),3.17 (q, 2H), 4.11 (q, 2H), 4.23 (s, 3H), 4.57 (s,2H), 4.68 (s, 2H), 7.48 (d, 1H), 7.57 (s, 1H),7.65 (s, 1H), 7.79 (s, 1H), 8.40 (s, 1H). |
| 5-4 | Cl | F | 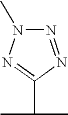 | Rf = 0.77[Hex: EA / 2:1] | 0.81-0.93 (m, 4H), 1.08 (t, 3H), 1.24 (t, 3H),1.43-1.53 (m, 1H), 1.63-1.75 (m, 5H), 2.14 (d,2H), 3.11 (d, 2H), 3.15 (q, 2H), 4.21 (s, 3H),4.63 (s, 2H), 4.70 (s, 2H), 7.44 (d, 1H), 7.50 (dd,1H), 7.56 (dd, 1H), 8.38 (d, 1H). |
| 5-5 | CF₃ | H | 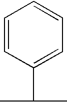 | Rf = 0.82[Hex: EA/ 9:1] | Mixture0.82-0.97 (m, 4H), 1.09 (t, 3H), 1.42-1.52 (m,1H), 1.63-1.77 (m, 5H), 2.14 (d, 2H), 3.13-3.18(m, 4H), 4.49 (s, 2H), 4.68 (s, 2H), 6.60-6.64 (m,2H), 6.75-6.84 (m, 1H), 7.17-7.23 (m, 2H), 7.64-7.67 (m, 2H), 7.79 (s, 1H), 7.84 (s, 1H), 8.42 (s,1H). |
| 5-6 | Cl | H | 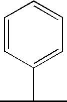 | Rf = 0.82[Hex: EA/ 9:1] | Mixture0.83-0.98 (m, 4H), 1.10 (t, 3H), 1.41-1.52 (m,1H), 1.64-1.77 (m, 5H), 2.15 (d, 2H), 3.14-3.19(m, 4H), 4.47 (s, 2H), 4.60 (s, 2H), 6.58-6.63 (m,2H), 6.74-7.00 (m, 1H), 7.16-7.22 (m, 2H), 7.36(s, 1H), 7.39 (s, 1H), 7.51 (s, 1H), 7.63 (s, 1H),8.42 (s, 1H). |
| 5-7 | CF₃ | H | 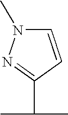 | 709[M + 1]⁺ | 0.8-0.93 (m, 4H), 1.01 (t, 3H), 1.24 (t, 3H), 1.38-1.5 (m, 1H), 1.6-1.8 (m, 5H), 2.13 (d, 2H), 3.08-3.18 (m, 4H), 3.75 (s, 3H), 4.11 (q, 2H), 4.4 (s,2H), 4.47 (s, 2H), 5.37 (d, 1H), 7.15 (d, 1H),7.67 (s, 2H), 7.72 (s, 2H), 8.36 (s, 1H) |
| 5-8 | Cl | H | 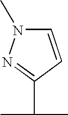 | 674[M + 1]⁺ | 0.8-0.93 (m, 4H), 1.02 (t, 3H), 1.24 (t, 3H), 1.38-1.5 (m, 1H), 1.6-1.75 (m, 5H), 2.13 (d, 2H), 3.07-3.17 (m, 4H), 3.76 (s, 3H), 4.11 (q, 2H), 4.39 (s,4H), 5.38 (d, 1H), 7.15 (d, 1H), 7.34 (s, 1H),7.38 (s, 1H), 7.45 (s, 1H), 7.72 (s, 1H), 8.36 (s,1H) |
| 5-9 | CF3 | H | 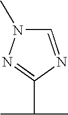 | 709[M + 1]⁺ | 0.75-0.9 (m, 4H), 1.03 (t, 3H), 1.24 (t, 3H), 1.38-1.5 (m, 1H), 1.6-1.7 (m, 5H), 2.12 (d, 2H), 3.08 (d,2H), 3.13 (q, 2H), 3.79 (s, 3H), 4.10 (q, 2H),4.55 (s, 2H), 4.62 (s, 2H), 7.57 (s, 1H), 7.61 (s,2H), 7.72 (s, 1H), 7.74 (s, 1H), 8.36(s, 1H) |
| 5-10 | Cl | H | 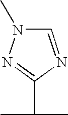 | 675[M + 1]⁺ | 0.8-0.95 (m, 4H), 1.03 (t, 3H), 1.24 (t, 3H), 1.4-1.8(m, 6H), 2.14 (d, 2H), 3.09 (d, 2H), 3.14 (q, 2H),3.8 (s, 3H), 4.12 (q, 2H), 4.48 (s, 2H), 4.59 (s, 2H),7.29 (s, 1H), 7.32 (s, 1H), 7.45 (s, 1H), 7.59 (s,1H), 7.74 (s, 1H), 8.37 (s, 1H) |
| 5-11 | Cl | H | 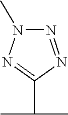 | 677[M + 1]⁺ | 0.83-0.91 (m, 4H), 1.05 (t, 3H), 1.24 (m, 4H),1.54-1.70 (m, 5H), 2.14 (d, 2H), 3.09 (d, 2H),3.12 (dd, 2H), 4.10 (q, 2H), 4.22 (s, 3H), 4.53 (s,2H), 4.64 (s, 2H), 7.31 (s, 1H), 7.48-7.50 (2H),8.39 (d, 1H). |

### Example 6

The following compounds are prepared from [4({[3-({[3,5-bis(trifluoromethyl)benzyl]amino}methyl)-5-(trifluoromethyl)pyridin-2-yl]ethylamino}methyl)cyclohexyl]acetic acid ethyl ester following the procedure of example 3 by use of appropriate bases and conditions.

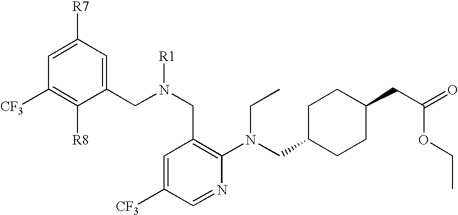

| No. | R₇ | R₈ | R₁ | MS | ¹H-NMR (400 MHz), δ (ppm) |
|---|---|---|---|---|---|
| 6-1 | CF₃ | H | 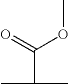 | 686[M + 1]⁺ | CDCl₃: 0.79-0.83 (m, 4H), 1.03 (t, 3H), 1.21 (t,3H), 1.36-1.45 (m, 1H), 1.50-1.67 (m, 5H), 2.12(d, 2H), 3.06 (d, 2H), 3.11 (q, 2H), 3.87 (s, 3H),4.10 (q, 2H), 4.33-4.54 (m, 4H), 7.40-7.65 (m,3H), 7.78 (s, 1H), 8.39 (d, 1H). |
| 6-2 | CF₃ | H | 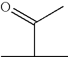 | 670[M + 1]⁺ | Isomer mixtureCDCl₃: 0.73-0.90 (m, 4H), 0.96 (t, 1.2H), 1.06 (t,1.8H), 1.22-1.28 (m, 3H), 1.33-1.75 (m, 6H),2.10 (d, 0.8H), 2.12 (d, 1.2H), 2.20 (s, 1.8H),2.28 (s, 1.2H), 3.00-3.15 (m, 4H), 4.10 (q, 2H),4.43 (s, 2H), 4.59 (s, 1.2H), 4.63 (s, 0.8H), 7.38(s, 0.6H), 7.52 (s, 1.2H), 7.62 (s, 1.2H), 7.78 (s,0.6H), 7.83 (s, 0.4H), 8.41 (s, 0.4H), 8.44 (s,0.6H). |
| 6-3 | CF₃ | H | 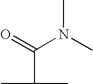 | 699[M + 1]⁺ | CDCl₃: 0.73-0.83 (m, 4H), 0.99 (t, 3H), 1.24 (t,3H), 1.36-1.45 (m, 1H), 1.50-1.67 (m, 5H), 2.11(d, 2H), 2.91 (s, 6H), 3.04 (d, 2H), 3.11 (q, 2H),4.12 (q, 2H), 4.30 (s, 2H), 4.32 (s, 2H), 7.60 (s,2H), 7.71 (d, 1H), 7.77 (s, 1H), 8.42 (d, 1H). |
| 6-4 | CF₃ | H | 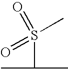 | 706[M + 1]⁺ | CDCl₃: 0.85-0.93 (m, 4H), 1.05 (t, 3H), 1.24 (t,3H), 1.36-1.45 (m, 1H), 1.64-1.89 (m, 5H), 2.14(d, 2H), 3.03 (s, 3H), 3.06 (d, 2H), 3.11 (q, 2H),4.10 (q, 2H), 4.31 (s, 2H), 4.45 (s, 2H), 7.48 (s,2H), 7.63 (d, 1H), 7.71 (s, 1H), 8.26 (d, 1H). |

### Example 7

### Synthesis of [trans-4-({[3-({[3,5-bis(trifluoromethyl)benzyl](2-methyl-2H-tetrazol-5-yl)amino}methyl)-5-phenylpyridin-2-yl]ethylamino}methyl)cyclohexyl]acetic acid ethyl ester

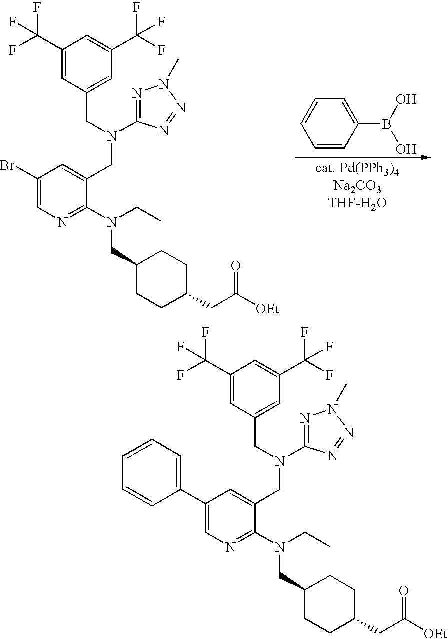

A mixture of [trans-4-({[3-({[3,5-bis(trifluoromethyl)benzyl](2-methyl-2H-tetrazol-5-yl)amino}methyl)-5-bromopyridin-2-yl]ethylamino}methyl)cyclohexyl]acetic acid ethyl ester (80 mg, 0.11 mmol), phenyl boronic acid (17 mg, 0.14 mmol), tetrakistriphenylphosphine palladium (12 mg, 0.01 mmol) and 2M sodium carbonate solution (210 μL, 0.42 mmol) in THF (2 mL) is stirred at 80° C. under argon atmosphere for 2 hours. After cooling to room temperature, the mixture is diluted with THF and filtered and then the filtrate is evaporated. The residue is purified by reverse phase HPLC (0.1% TFA to CH₃CN) to give [trans-4-({[3-({[3,5-bis(trifluoromethyl)benzyl](2-methyl-2H-tetrazol-5-yl)amino}methyl)-5-phenylpyridin-2-yl]ethylamino}methyl)cyclohexyl]acetic acid ethyl ester (40 mg, 51%) as colorless oil.

¹H-NMR (400 MHz, CDCl₃), δ (ppm): 0.82-0.92 (m, 4H), 1.03 (t, 3H), 1.24 (t, 3H), 1.45-1.55 (m, 1H), 1.54-1.80 (m, 5H), 2.12 (d, 2H), 3.03 (d, 2H), 3.09 (q, 2H), 4.10 (q, 2H), 4.20 (s, 3H), 4.64 (s, 2H), 4.79 (s, 2H), 7.32-7.41 (m, 5H), 7.56 (d, 1H), 7.64 (s, 2H), 7.72 (s, 1H), 8.45 (d, 1H).

ESI-MS m/z: 718 [M+1]⁺

### Example 8

The following compounds are prepared from [trans-4-({[3-({[3,5-bis(trifluoromethyl)benzyl](2-methyl-2H-tetrazol-5-yl)amino}methyl)-5-bromopyridin-2-yl]ethylamino}-methyl)cyclohexyl]acetic acid ethyl ester following the procedure of example 7 utilizing appropriate reagents and conditions.

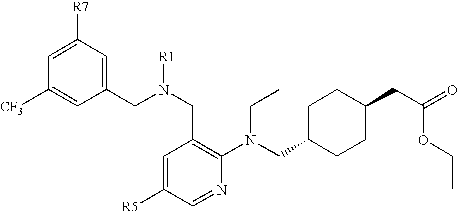

| | | | | MS or | |
|---|---|---|---|---|---|
| | | | | Rf | ¹H-NMR (400 MHz, CDCl₃), δ (ppm) or |
| No. | R₇ | R₁ | R5 | value | HPLC/UPLC Retention time |
| 8-1 | CF₃ | 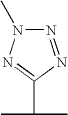 | Pyridine-3-yl | 719[M + 1]⁺ | 0.80-0.90 (m, 4H), 1.05 (t, 3H), 1.24 (t, 3H), 1.45-1.80 (m, 6H), 2.12 (d, 2H), 3.06 (d, 2H), 3.11 (q,2H), 4.10 (q, 2H), 4.21 (s, 3H), 4.64 (s, 2H), 4.78(s, 2H), 7.31-7.34 (m, 1H), 7.55 (d, 1H), 7.62 (s,2H), 7.66-7.69 (m, 1H), 7.72 (s, 1H), 8.43 (d, 1H),8.56 (d, 1H), 8.67 (d, 1H). |

### Example 9

### Synthesis of [5-({[3,5-bis(trifluoromethyl)benzyl](2-methyl-2H-tetrazol-5-yl)amino}methyl)-[3,3]bipyridinyl-6-yl](cyclopentylmethyl)ethylamine

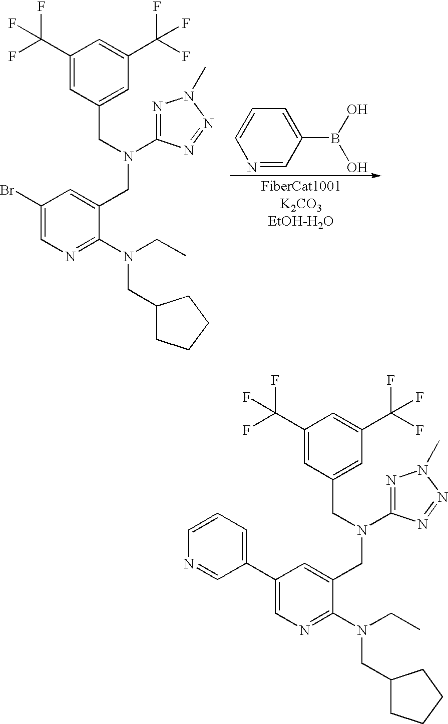

A mixture of [3-({[3,5-bis(trifluoromethyl)benzyl](2-methyl-2H-tetrazol-5-yl)amino}methyl)-5-bromopyridin-2-yl](cyclopentylmethyl)ethylamine (141 mg, 0.23 mmol), K₂CO₃ (94 mg, 0.68 mmol) and FibreCat® 1001 (35 mg, 0.011 mmol, CAS: 457645-05-5) in EtOH/H₂O (10:1, 1.2 mL) is heated at 80° C. overnight. After cooling down to room temperature, the reaction mixture is filtered and diluted with DMSO. H₂O is added to the reaction mixture. The mixture is extracted with EtOAc. The filtrate is purified by reverse phase preparative HPLC and silica gel flash chromatography to give [5-({[3,5-bis(trifluoromethyl)benzyl](2-methyl-2H-tetrazol-5-yl)amino}methyl)-[3,3]bipyridinyl-6-yl](cyclopentylmethyl)ethylamine as pale yellow oil (55 mg, 0.089 mmol; 39%); ESI-MS m/z: 619 [M+1]⁺, Retention time: 1.95 min.

### Example 10

### Synthesis of [3-({[3,5-bis(trifluoromethyl)benzyl](2-methyl-2H-tetrazol-5-yl)amino}methyl)-5-(furan-2-yl)pyridin-2-yl](cyclopentylmethyl)ethylamine

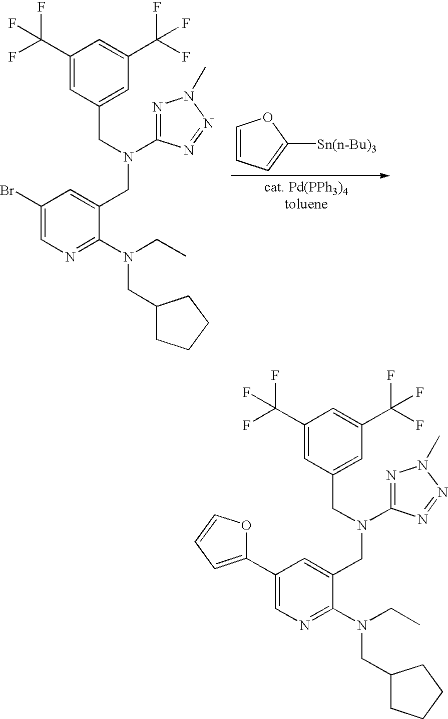

A mixture of [3-({[3,5-bis(trifluoromethyl)benzyl](2-methyl-2H-tetrazol-5-yl)amino}methyl)-5-bromopyridin-2-yl](cyclopentylmethyl)ethylamine (155 mg, 0.25 mmol), tributyl(2-furyl)stannane (107 mg, 0.30 mmol, CAS: 118486-94-51) and Pd(PPh₃)₄ (29 mg, 0.025 mmol,) in toluene (2.0 mL) is heated at 120° C. overnight. After cooling down to room temperature, the reaction mixture is diluted with EtOAc. The organic layer is washed with 10% NaF solution and brine, dried and concentrated under reduced pressure. The resulting residue is purified by silica gel flash chromatography to give [3-({[3,5-bis(trifluoromethyl)benzyl](2-methyl-2H-tetrazol-5-yl)amino}methyl)-5-(furan-2-yl)pyridin-2-yl](cyclopentylmethyl)ethylamine as pale yellow oil (129 mg, 0.21 mmol; 85%); ESI-MS m/z: 608 [M+1]⁺, Retention time: 2.13 min.

### Example 11

### Synthesis of [3-({[3,5-bis(trifluoromethyl)benzyl](2-methyl-2H-tetrazol-5-yl)amino}methyl)-5-(pyrrol-1-yl)pyridin-2-yl](cyclopentylmethyl)ethylamine

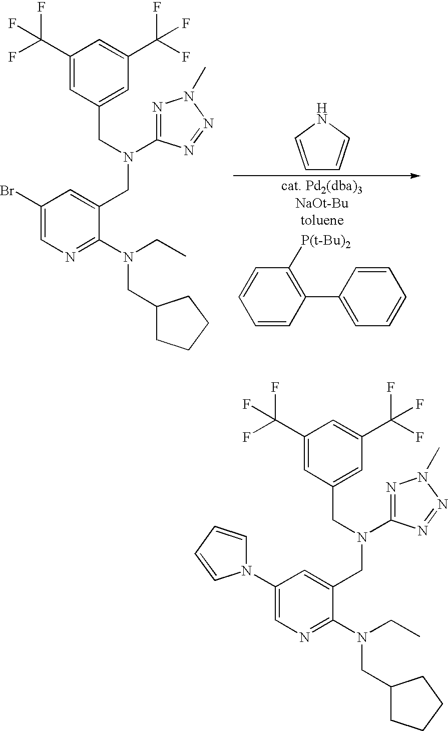

A mixture of [3-({[3,5-bis(trifluoromethyl)benzyl](2-methyl-2H-tetrazol-5-yl)amino}methyl)-5-bromopyridin-2-yl](cyclopentylmethyl)ethylamine (152 mg, 0.25 mmol), sodium tert-butoxide (35 mg, 0.36 mmol), pyrrole (33 mg, 0.49 mmol), Pd₂(dba)₃ (22 mg, 0.024 mmol,) and 2-(di-tert-butylphosphino)biphenyl (7.0 mg, 0.024 mmol, CAS: 224311-51-7) in toluene (2.0 mL) is heated at 80° C. for 2 h. H₂O is added to the reaction mixture. After filtration through Celite®, the mixture is extracted with EtOAc. The organic layer is washed with H₂O, dried and concentrated under reduced pressure. The resulting residue is purified by silica gel flash chromatography to give [3-({[3,5-bis(trifluoromethyl)benzyl](2-methyl-2H-tetrazol-5-yl)amino}methyl)-5-(pyrrol-1-yl)pyridin-2-yl](cyclopentylmethyl)ethylamine as pale yellow oil (61 mg, 0.10 mmol; 41%); ESI-MS m/z: 607 [M+1]⁺, Retention time: 2.28 min.

### Example 12

The following compounds are prepared following the procedure of Example 9-11.

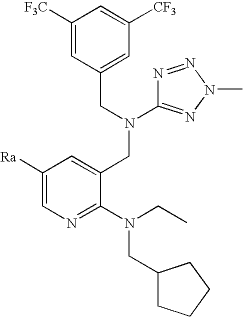

| | | | UPLC Retention |
|---|---|---|---|
| No. | Ra | MS | time |
| 12-1 | 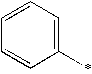 | 618[M + 1]⁺ | 2.08 min (UPLC). |
| 12-2 | 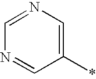 | 620[M + 1]⁺ | 2.08 min (UPLC). |
| 12-3 | 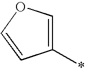 | 608[M + 1]⁺ | 2.04 min (UPLC). |
| 12-4 | 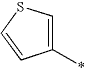 | 624[M + 1]⁺ | 2.09 min (UPLC). |
| 12-5 | 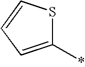 | 624[M + 1]⁺ | 2.18 min (UPLC). |
| 12-6 | 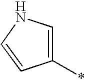 | 607[M + 1]⁺ | 1.98 min (UPLC). |

### Example 13

The following compounds are prepared from trans-4-{[(5-halo-3-hydroxymethyl-pyridin-2-yl)ethyl-amino]methyl}cyclohexyl)acetic acid ethyl ester following the procedure of example 2.

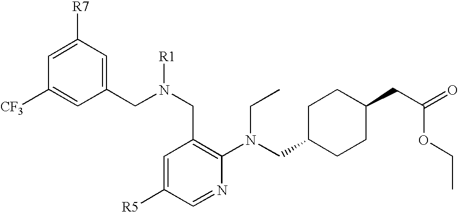

| | | | | MS or Rf | ¹H-NMR (400 MHz, CDCl₃), δ (ppm) or HPLC/UPLC |
|---|---|---|---|---|---|
| No. | R₇ | R₁ | R5 | value | Retention time |
| 13-1 | CF₃ | 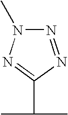 | Cl | 676[M + 1]⁺ | 0.80-0.87 (m, 4H), 0.98 (t, 3H), 1.24 (t, 3H), 1.48-1.53(m, 1H), 1.64-1.66 (m, 4H), 2.12 (d, 2H), 2.95 (d, 2H),3.01 (dd, 2H), 4.10 (q, 2H), 4.21 (s, 3H), 4.64 (s, 2H),4.68 (s, 2H), 7.32 (d, 1H), 7.63 (s, 2H), 7.70 (s, 1H),8.13 (d, 1H). |
| 13-2 | CF₃ | 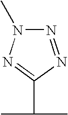 | Br | 720, 722[M + 1]⁺ | 0.79-0.87 (m, 4H), 0.99 (t, 3H), 1.25 (t, 3H), 1.48-1.53(m, 1H), 1.64-1.66 (m, 5H), 2.12 (d, 2H), 2.96 (d, 2H),3.02 (dd, 2H), 4.10 (q, 2H), 4.21 (s, 3H), 4.64 (s, 2H),4.67 (s, 2H), 7.44 (d, 1H), 7.63 (s, 2H), 7.77 (s, 1H),8.21 (d, 1H). |

### Example 14

### Synthesis of [trans-4-({[3-({[3,5-bis(trifluoromethyl)benzyl](2-methyl-2H-tetrazol-5-yl)amino}methyl)-5-(trifluoromethyl)pyridin-2-yl]ethylamino}methyl)cyclohexyl]acetic acid

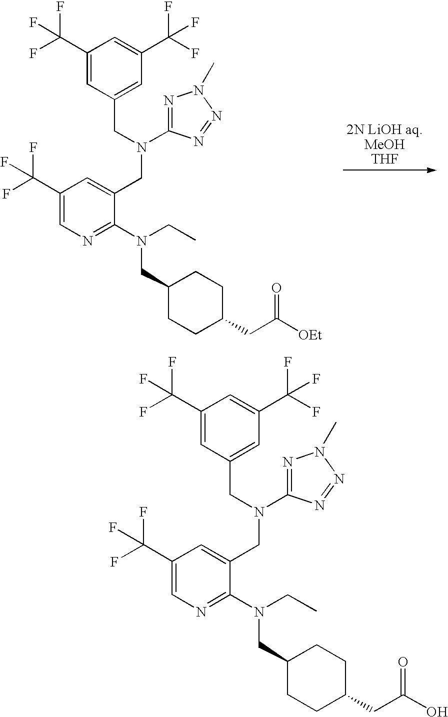

To a solution of [trans-4-({[3-({[3,5-bis(trifluoromethyl)benzyl](2-methyl-2H-tetrazol-5-yl)amino}methyl)-5-(trifluoromethyl)pyridin-2-yl]ethylamino}methyl)cyclohexyl]acetic acid ethyl ester (1.22 g, 1.72 mmol) in THF-MeOH (7:3, 10.0 mL) is added 2N LiOH (5.1 mL) and the mixture is stirred at room temperature for 16 hours. The mixture is diluted with 1N HCl and ethyl acetate, and the organic layer is washed with brine, dried over magnesium sulfate, filtered and concentrated. The residue is purified by silica gel column chromatography to give [trans-4-({[3-({[3,5-bis(trifluoromethyl)benzyl](2-methyl-2H-tetrazol-5-yl)amino}methyl)-5-trifluoromethylpyridin-2-yl]ethylamino}methyl)cyclohexyl]acetic acid (0.84 g, 70% yield).

¹H-NMR (400 MHz, CDCl₃), δ (ppm): 0.81-0.94 (m, 4H), 1.05 (t, 3H) 1.43-1.53 (m, 1H), 1.63-1.75 (m, 5H), 2.18 (d, 2H), 3.10 (d, 2H), 3.15 (q, 2H), 4.22 (s, 3H), 4.61 (s, 2H), 4.68 (s, 2H), 7.49 (d, 1H), 7.59 (s, 2H), 7.75 (s, 1H), 8.39 (d, 1H).

ESI-MS m/z: 682 [M+1]⁺

### Example 15

The following compounds are prepared from corresponding esters following the procedure of example 13.

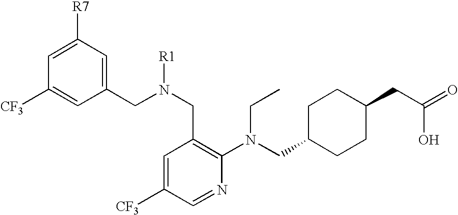

| No. | R₇ | R₈ | R₁ | MS | ¹H-NMR (400 MHz), δ (ppm) |
|---|---|---|---|---|---|
| 15-1 | Cl | H | 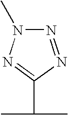 | 648[M + 1]⁺ | 0.84-0.91 (m, 4H), 1.05 (t, 3H), 1.45-1.53 (m,3H), 1.65-1.76 (m, 6H), 2.19 (d, 2H), 3.09 (d,2H), 3.15 (dd, 2H), 4.22 (s, 3H), 4.53 (s, 2H),4.65 (s, 2H), 7.31 (s, 1H), 7.48 (s, 1H), 7.50(d, 1H), 8.39 (d, 1H). |
| 15-2 | NO₂ | H | 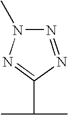 | 659[M + 1]⁺ | CDCl₃: 0.82-0.89 (m, 4H), 1.05 (t, 3H), 1.26(t, 3H),1.43-1.49 (m, 1H), 1.50-1.90 (m, 5H),2.15 (d, 2H), 3.11 (d, 2H), 3.16 (q, 2H), 4.23(s, 3H), 4.72 (s, 2H), 7.50 (d, 1H), 7.72 (s,1H), 8.19 (s, 1H), 8.36 (s, 1H), 8.38 (s, 1H). |
| 15-3 | CN | H | 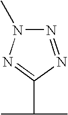 | 639[M + 1]⁺ | CDCl₃: 0.81-0.94 (m, 4H), 1.05 (t, 3H) 1.43-1.53 (m, 1H), 1.63-1.75 (m, 5H), 2.18 (d, 2H),3.10 (d, 2H), 3.15 (q, 2H), 4.22 (s, 3H), 4.61(s, 2H), 4.68 (s, 2H), 7.49 (d, 1H), 7.59 (s,2H), 7.75 (s, 1H), 8.44 (s, 1H). |
| 15-4 | Cl | F | 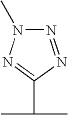 | 667[M + 1]⁺ | CDCl₃: 0.82-0.98 (m, 4H), 1.09 (t, 3H), 1.47-1.59 (m, 1H), 1.62-1.81 (m, 5H), 2.22 (d, 2H),3.16 (d, 2H), 3.24 (q, 2H), 4.23 (s, 3H), 4.61(s, 2H), 4.67 (s, 2H), 7.56 (d, 1H), 7.61 (s,1H), 7.66 (s, 1H), 7.81 (s, 1H), 8.38 (s, 1H). |
| 15-5 | CF₃ | H | 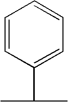 | 676[M + 1]⁺ | CDCl₃: 0.87-0.93 (m, 4H), 1.09 (t, 3H), 1.26(t, 3H), 1.23-1.34 (m, 1H), 1.65-1.80 (m, 5H),2.20 (d, 2H), 3.15 (q, 4H), 4.49 (s, 2H), 4.67(s, 2H), 6.63 (d, 2H), 6.82 (t, 1H), 7.21 (dd,2H), 7.65-7.68 (m, 3H), 7.79 (s, 1H), 8.42 (s,1H). |
| 15-6 | Cl | H | 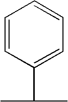 | 642[M⁺] | CDCl₃: 0.86-0.93 (m, 4H), 1.09 (t, 3H), 1.30-1.57 (m, 1H), 1.63-1.82 (m, 5H), 2.18 (d, 2H),3.14-3.16 (m, 4H), 4.47 (s, 2H), 4.60 (s, 2H),6.62 (d, 2H), 6.80 (t, 1H), 7.20 (dd, 2H), 7.36(s, 1H), 7.39 (s, 1H), 7.51 (s, 1H), 7.64 (d,1H), 8.42 (d, 1H). |
| 15-7 | CF₃ | H | 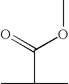 | 658[M + 1]⁺ | CDCl₃: 0.75-0.95 (m, 4H), 1.03 (t, 3H), 1.42-1.80 (m, 6H), 2.18 (d, 2H), 3.06 (d, 2H), 3.11(q, 2H), 3.87 (s, 3H), 4.35-4.55 (m, 4H), 7.44-7.60 (m, 3H), 7.77 (s, 1H), 8.39 (d, 1H). |
| 15-8 | CF₃ | H | 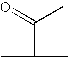 | 642[M + 1]⁺ | mixture of isomersCDCl₃: 0.68-1.02 (m, 4H), 0.96 (t,1.2H), 1.06 (t, 1.8H), 1.22-1.28 (m, 3H), 1.35-1.93 (m, 6H), 2.15-2.20 (m, 2H), 2.21 (s,1.8H), 2.28 (s, 1.2H), 3.01-3.14 (m, 4H), 4.10(q, 2H), 4.43 (s, 2H), 4.59 (s, 1.2H), 4.64 (s,0.8H), 7.38 (s, 0.6H), 7.52-7.53 (m, 1.2H),7.62 (s, 1.2H), 7.78 (s, 0.6H), 7.83 (s, 0.4H),8.42 (s, 0.4H), 8.44 (s, 0.6H). |
| 15-9 | CF₃ | H | 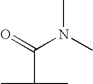 | 671[M + 1]⁺ | CDCl₃: 0.70-0.85 (m, 4H), 0.99 (t, 3H), 1.36-1.75 (m, 6H), 2.17 (d, 2H), 2.91 (s, 6H), 3.04(d, 2H), 3.10 (q, 2H), 4.30 (s, 2H), 4.32 (s,2H), 7.59 (s, 2H), 7.72 (d, 1H), 7.77 (s, 1H),8.43 (d, 1H). |
| 15-10 | CF₃ | H | 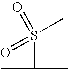 | 678[M + 1]⁺ | CDCl₃: 0.83-0.98 (m, 4H), 1.05 (t, 3H), 1.66-1.83 (m, 5H), 2.20 (d, 2H), 3.03 (s, 3H), 3.04(d, 2H), 3.10 (q, 2H), 4.31 (s, 2H), 4.46 (s,2H), 7.48 (s, 2H), 7.63 (d, 1H), 7.71 (s, 1H),8.27 (s, 1H). |
| 15-11 | CF₃ | H | 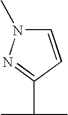 | 680[M + 1]⁺ | 0.78-0.92 (m, 4H), 1.03 (t, 3H), 1.4-1.8 (m, 6H),2.18 (d, 2H), 2.98-3.18 (m, 4H), 3.76 (s, 3H),4.4 (q, 2H), 4.47 (s, 4H), 5.37 (d, 1H), 7.15 (d,1H), 7.66 (s, 2H), 7.72 (s, 2H), 8.37 (s, 1H) |
| 15-12 | Cl | H | 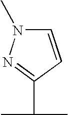 | 646[M + 1]⁺ | 0.78-0.92 (m, 4H), 1.04 (t, 3H), 1.4-1.8 (m, 6H),2.19 (d, 2H), 3.08-3.18 (m, 4H), 3.76 (s, 3H),4.38 (s, 2H), 4.39 (s, 2H), 5.38 (d, 1H), 7.15 (d,1H), 7.33 (s, 1H), 7.37 (s, 1H), 7.45 (s, 2H),7.72 (s, 2H), 8.37 (s, 1H) |
| 15-13 | CF₃ | H | 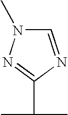 | 681[M + 1]⁺ | 0.76-0.9 (m, 4H), 1.03 (t, 3H), 1.4-1.8 (m, 6H),2.17 (d, 2H), 3.08 (d, 2H), 3.14 (q, 2H), 3.79 (s,3H), 4.55 (s, 2H), 4.62 (s, 2H), 7.58 (s, 1H),7.61 (s, 2H), 7.72 (s, 1H), 7.76 (s, 1H), 8.36 (s,1H) |
| 15-14 | Cl | H | 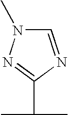 | 647[M + 1]⁺ | 0.8-0.95 (m, 4H), 1.03 (t, 3H), 1.4-1.8 (m, 6H),2.19 (d, 2H), 3.08 (d, 2H), 3.12 (q, 2H), 3.8 (s,3H), 4.47 (s, 2H), 4.59 (s, 2H), 7.28 (s, 1H),7.32 (s, 1H), 7.45 (s, 1H), 7.59 (s, 1H), 7.75 (s,1H), 8.37 (s, 1H) |

### Example 16

The following compounds are prepared from the corresponding esters by hydrolysis following the procedure of example 14.

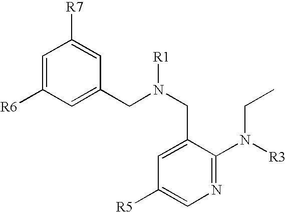

| | | | | | | | ¹H-NMR (400 MHz, CDCl₃), δ |
|---|---|---|---|---|---|---|---|
| No. | R7 | R6 | R1 | R3 | R5 | MS | (ppm) |
| 16-1 | CF₃ | CF₃ | 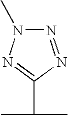 | 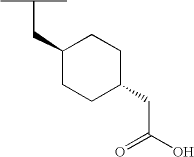 | Br | 692, 694[M + 1]⁺ | 0.79-0.92 (m, 4H), 0.99 (t, 3H),1.45-1.80 (m, 6H), 2.17 (d, 2H),2.96 (d, 2H), 3.02 (q, 2H), 4.21(s, 3H), 4.64 (s, 2H), 4.67 (s, 2H),7.44 (d, 1H), 7.63 (s, 2H), 7.77(s, 1H), 8.22 (d, 1H). |
| 16-2 | CF₃ | CF₃ | 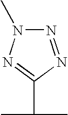 | 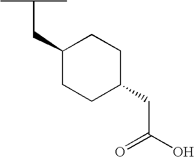 | Ph | 690[M + 1]⁺ | 0.80-0.92 (m, 4H), 1.03 (t, 3H),1.45-1.80 (m, 6H), 2.18 (d, 2H),3.04 (d, 2H), 3.09 (q, 2H), 4.20(s, 3H), 4.65 (s, 2H), 4.80 (s, 2H),7.26-7.45 (m, 5H), 7.57 (d, 1H),7.64 (s, 2H), 7.72 (s, 1H), 8.45(d, 1H). |
| 16-3 | CF₃ | CF₃ | 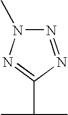 | 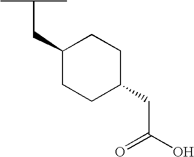 | Pyridine-3-yl | 691[M + 1]⁺ | 0.82-0.92 (m, 4H), 1.05 (t, 3H),1.45-1.80 (m, 6H), 2.18 (d, 2H),3.07 (d, 2H), 3.13 (q, 2H), 4.21(s, 3H), 4.64 (s, 2H), 4.78 (s, 2H),7.34-7.37 (m, 1H), 7.56 (d, 1H),7.62 (s, 2H), 7.71-7.73 (m, 2H),8.44 (d, 1H), 8.57 (d, 1H), 8.68(d, 1H). |
| 16-4 | CF₃ | CF₃ | 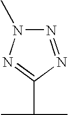 | 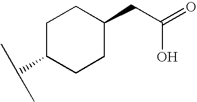 | Cl | 648[M + 1]⁺ | 0.75-0.92 (m, 4H), 0.99 (t, 3H),1.57-1.71 (m, 5H), 2.17 (d, 2H),2.95 (d, 2H), 3.01 (dd, 2H), 3.71(s, 1H), 4.21 (s, 3H), 4.64 (s, 2H),4.68 (s, 2H), 7.32 (d, 2H), 7.63(s, 2H), 7.77 (s, 1H), 8.13 (d, 1H). |
| 16-5TAK334 | CF₃ | CF₃ | 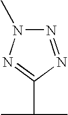 | 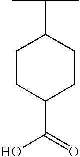 | CF₃ | 654[M + 1]⁺ | 2.27 min (UPLC) |
| 16-6TAK468 | CF₃ | CF₃ | 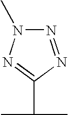 | 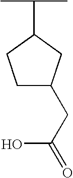 | CF₃ | 654[M + 1]⁺ | 2.26 min (UPLC) |

### Example 17

### Preparation of [trans-4-({[3-({[3,5-bis(trifluoromethyl)benzyl](2-methyl-2H-tetrazol-5-yl)amino}methyl)-5-trifluoromethylpyridin-2-yl]ethylamino}methyl)cyclohexyl]-acetamide

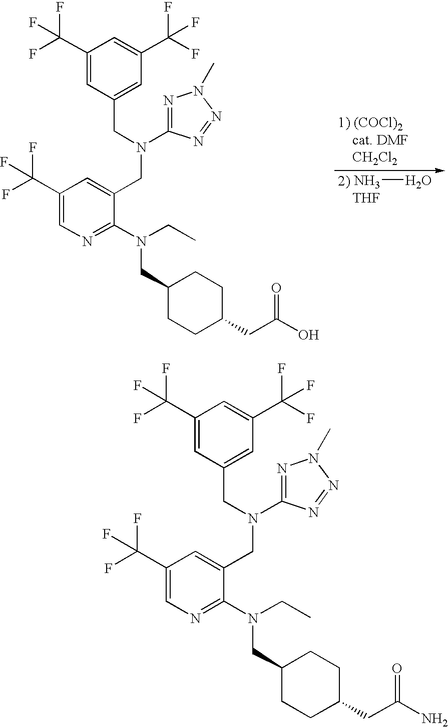

A mixture of [trans-4-({[3-({[3,5-bis(trifluoromethyl)benzyl](2-methyl-2H-tetrazol-5-yl)amino}-methyl)-5-trifluoromethylpyridin-2-yl]ethylamino}methyl)cyclohexyl]acetic acid (68 mg, 0.10 mmol), oxalyl chloride and catalytic amount of DMF in dichloromethane is stirred at ambient temperature for 2 hours. After the mixture is concentrated in vacuo, 2 mL of THF is added. To the solution, ammonia solution (1 mL) in THF (1 mL) is added. After stirring at ambient temperature for 1 hour, ethyl acetate and water are added, and partitioned. The combined organic layer is washed with brine, dried over magnesium sulfate, filtrated and concentrated. The residue is purified by reverse phase HPLC (0.1% TAF-H₂O to CH₃CN) to give [trans-4-({[3-({[3,5-bis(trifluoromethyl)benzyl](2-methyl-2H-tetrazol-5-yl)amino}methyl)-5-trifluoromethylpyridin-2-yl]ethylamino}methyl)cyclohexyl]acetamide.

¹H-NMR (400 MHz, CDCl₃), δ (ppm): 0.83-0.92 (m, 4H), 1.05 (t, 3H), 1.45-1.80 (m, 6H), 2.04 (d, 2H), 3.10 (d, 2H), 3.15 (q, 2H), 4.22 (s, 3H), 4.61 (s, 2H), 4.69 (s, 2H), 5.31 (brs, 2H), 7.48 (d, 1H), 7.59 (s, 2H), 7.75 (s, 1H), 8.38 (d, 1H). ESI-MS m/z: 681 [M+1]⁺

### Example 18

### Preparation of trans-2-(4-({[(3-({[(3,5-bis(trifluoromethyl)benzyl](2-methyl-2H-tetrazol-5-yl)-amino}methyl)-5-trifluoromethyl-pyridin-2-yl]ethylamino}methyl)-cyclohexyl)-ethanol

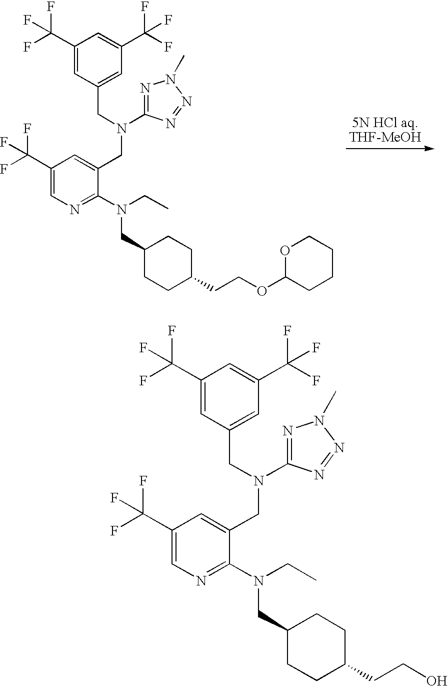

A mixture of trans-[3-({[3,5-bis(trifluoromethyl)benzyl](2-methyl-2H-tetrazol-5-yl)amino}methyl)-5-trifluoromethylpyridin-2-yl](ethyl){4-[2-(tetrahydropyran-2-yloxy)ethyl]cyclohexylmethyl}amine (1.10 g, 1.5 mmol), 5N HCl aq (1.6 mL) in THF-MeOH [8:1, 9 mL] is stirred for 18 hours at ambient temperature. After addition of sat. NaHCO₃ aqueous solution, the mixture is extracted with ethyl acetate. The organic layer is washed with brine, dried over magnesium sulfate, filtrated and concentrated to give trans-2-[4-({[3-({[3,5-bis(trifluoro-methyl)benzyl](2-methyl-2H-tetrazol-5-yl)amino}methyl)-5-trifluoromethyl-pyridin-2-yl]ethylamino}methyl)cyclohexyl]ethanol (0.87 g, 89% yield).

¹H-NMR (400 MHz, CDCl₃), δ (ppm): 0.75-0.87 (m, 4H), 1.05 (t, 3H), 1.13 (t, 1H), 1.15-1.25 (m, 1H), 1.43 (q, 2H), 1.43-1.53 (m, 1H), 1.60-1.70 (m, 4H), 3.09 (d, 2H), 3.16 (q, 2H), 3.62-3.69 (m, 2H), 4.22 (s, 3H), 4.60 (s, 2H), 4.69 (s, 2H), 7.49 (d, 1H), 7.60 (s, 2H), 7.76 (s, 1H), 8.39 (d, 1H). ESI-MS m/z: 668 [M+1]⁺

### Example 19

The following compounds are prepared by use of appropriate starting materials, reagents and conditions following the procedure of Example 18.

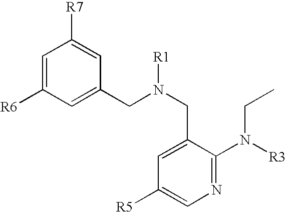

| No. | R7 | R6 | R₁ | R3 | R5 | MS | ¹H-NMR (400 MHz), δ (ppm) |
|---|---|---|---|---|---|---|---|
| 19-1 | CF₃ | CN | 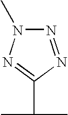 | 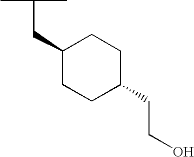 | CF₃ | 625[M + 1]⁺ | CDCl₃: 0.80-0.91 (m, 4H), 1.06(t, 3H), 1.18 (t, 1H), 1.24-1.38 (m,1H), 1.46 (q, 2H), 1.60-1.78 (m,5H), 3.10 (d, 2H), 3.17 (q, 2H),3.66 (q, 2H), 4.23 (s, 3H), 4.57 (s,2H), 4.68 (s, 2H), 4.49 (d, 1H),7.60 (s, 1H), 7.64 (s, 1H), 7.79 (s,1H), 8.40 (s, 1H). |

### Example 20

### Preparation of trans-[4-(2-aminoethyl)cyclohexylmethyl][3-({[3,5-bis(trifluoromethyl)benzyl](2-methyl-2H-tetrazol-5-yl)amino}methyl)-5-trifluoromethylpyridin-2-yl]ethylamine

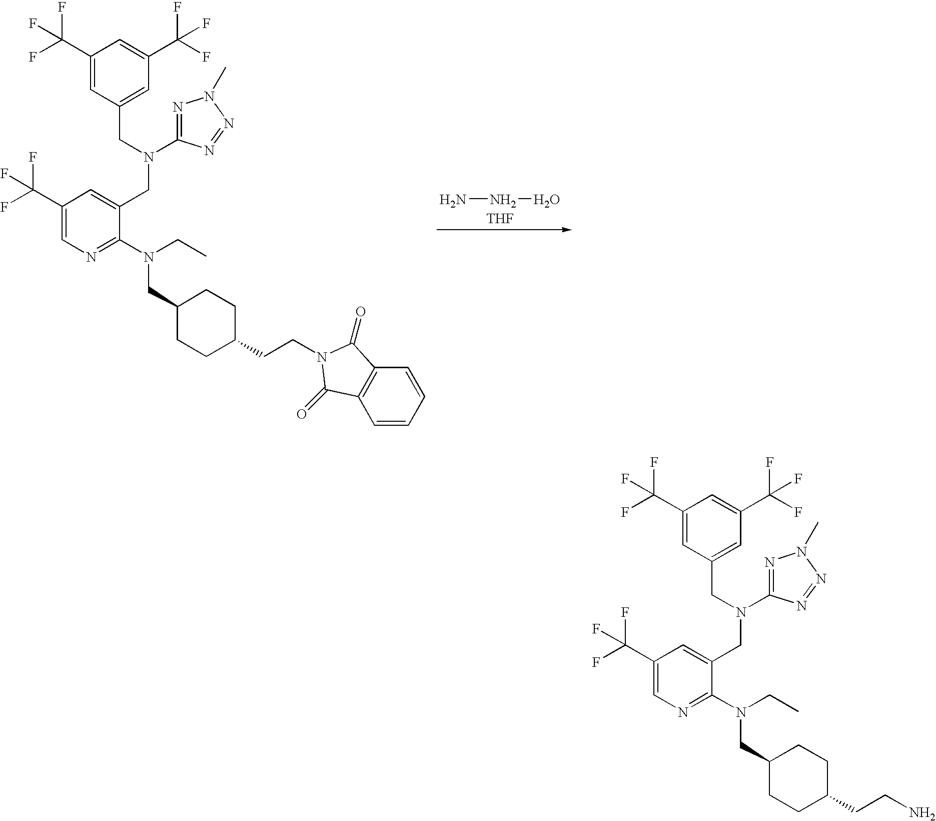

To a solution of trans-2-{2-[4-({[3-({[3,5-bis(trifluoromethyl)benzyl](2-methyl-2H-tetrazol-5-yl)amino}methyl)-5-trifluoromethylpyridin-2-yl]ethylamino}methyl)cyclohexyl]ethyl}isoindole-1,3-dione (0.22 g, 0.28 mmol) in THF (5 mL), hydrazine hydrate (0.309) is added and stirred at 50° C. for 4 hours. After adding hydrazine hydrate (0.30 g), the mixture is further stirred at 50° C. for 12 hours. The mixture is filtered and the filtrate is concentrated in vacuo. The residue is purified by reverse phase HPLC (0.1% TFA-H₂O to CH₃CN). After The residue is dissolved in ethyl acetate, the mixture is washed with sat. sodium bicarbonate and the brine, dried over magnesium sulfate, filtrated and concentrated in vacuo to give trans-[4-(2-aminoethyl)cyclohexylmethyl][3-({[3,5-bis(trifluoromethyl)benzyl](2-methyl-2H-tetrazol-5-yl)amino}methyl)-5-trifluoromethylpyridin-2-yl]ethylamine (0.086 g, 47% yield).

¹H-NMR (400 MHz, CDCl₃), δ (ppm): 0.75-0.86 (m, 4H), 1.05 (t, 3H), 1.15-1.25 (m, 1H), 1.35 (q, 2H), 1.45-1.75 (m, 5H), 2.72 (t, 2H), 3.08 (d, 2H), 3.16 (q, 2H), 4.22 (s, 3H), 4.60 (s, 2H), 4.68 (s, 2H), 7.49 (d, 1H), 7.59 (s, 2H), 7.75 (s, 1H), 8.38 (d, 1H). ESI-MS m/z: 667 [M+1]⁺

### Example 21

### Synthesis of [3-({[3,5-bis(trifluoromethyl)benzyl](2-methyl-2H-tetrazol-5-yl)amino}methyl)-5-trifluoromethylpyridin-2-yl]cyclopentylethylamine

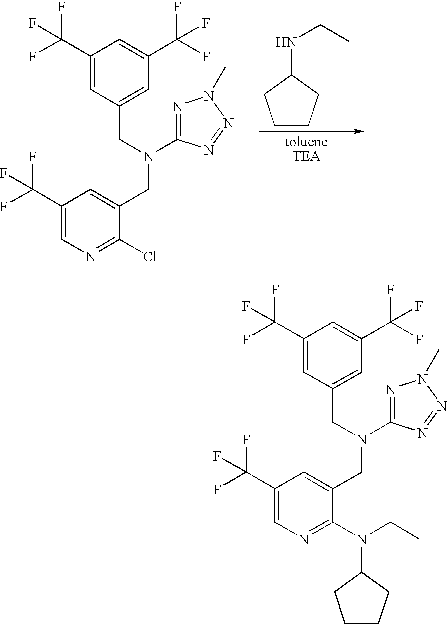

A suspension of [3,5-bis(trifluoromethyl)benzyl](2-chloro-5-trifluoromethylpyridin-3-ylmethyl)(2-methyl-2H-tetrazol-5-yl)amine (100 mg, 0.19 mmol), cyclopenthylethylamine (43 mg, 0.38 mmol), triethylamine (TEA; 270 μL) in toluene (1 mL) is stirred at 150° C. for 4 days in sealed tube. The reaction mixture is cooled to room temperature, diluted with water and dichloromethane. The organic layer is filtered through phase separator and concentrated. The crude product is purified by silica gel column chromatography to give [3-({[3,5-bis(trifluoromethyl)benzyl](2-methyl-2H-tetrazol-5-yl)amino}methyl)-5-trifluoromethylpyridin-2-yl]cyclopentylethylamine (8 mg, 7%).

¹H-NMR (400 MHz, CDCl₃), δ (ppm): 0.92 (t, 3H), 1.40-1.60 (m, 4H), 1.61-1.68 (m, 2H), 1.72-1.82 (m, 2H), 3.23 (dd, 2H), 3.65-3.75 (m, 1H), 4.22 (s, 3H), 4.60 (s, 2H), 4.71 (s, 2H), 7.59 (d, 1H), 7.65 (s, 2H), 7.77 (s, 1H), 8.45 (s, 1H).

ESI-MS m/z: 596 [M+1]⁺.

### Example 22

### Synthesis of [3-({[3,5-bis(trifluoromethyl)benzyl](2-methyl-2H-tetrazol-5-yl)amino}methyl)-5-trifluoromethylpyridin-2-yl]cyclohexylmethylethylamine

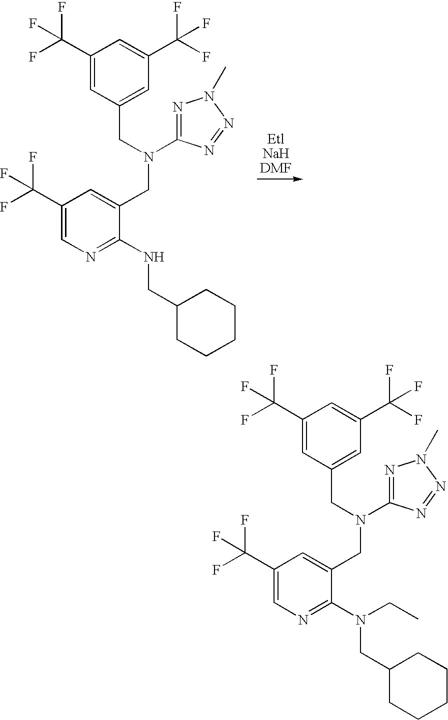

A mixture of [3-({[3,5-bis(trifluoromethyl)benzyl](2-methyl-2H-tetrazol-5-yl)amino}methyl)-5-trifluoromethylpyridin-2-yl]cyclohexylmethylamine (50 mg, 0.084 mmol), sodium hydride (60% dispersion in mineral oil; 4 mg, 0.10 mmol) and ethyl iodide (8.0 μL, 0.10 mmol) in DMF (0.50 mL) is stirred at ambient temperature for 2 hours. After addition of sodium hydride (60% dispersion in mineral oil; 40 mg, 1.0 mmol) and ethyl iodide (80 μL, 1.0 mmol), the mixture is stirred at 70° C. for 3 hours. After cooling to room temperature, sat. ammonium chloride is added to the mixture and the mixture is extracted with dichloromethane. The organic layer is filtrated through phase separator and concentrated. The resulting mixture is purified by silica gel column chromatography to give [3-({[3,5-bis(trifluoromethyl)benzyl](2-methyl-2H-tetrazol-5-yl)amino}methyl)5-trifluoromethylpyridin-2-yl]cyclohexylmethylethylamine (24 mg, 46% yield).

¹H-NMR (400 MHz, CDCl3), δ (ppm): 0.70-0.83 (m, 2H), 1.05 (t, 3H), 1.21-1.02 (m, 4H), 1.61-1.70 (m, 5H), 3.08 (d, 2H), 3.17 (dd, 2H), 4.22 (s, 3H), 4.60 (s, 2H), 4.68 (s, 2H), 7.49 (d, 1H), 7.60 (s, 2H), 7.75 (s, 1H), 8.39 (d, 1H). ESI-MS m/z: 624 [M+1]⁺.

### Example 23

The following compounds are prepared from [2-(substituted amino)-5-(substituted)pyridin-3-yl]methanol and [3-(trifluoromethyl)-5-(substituted)benzyl])(2-methyl-2H-tetrazol-5-yl)amine following the procedure of example 1 or [3,5-bis(trifluoromethyl)benzyl](2-chloro-5-trifluoromethylpyridin-3-ylmethyl)(2-methyl-2H-tetrazol-5-yl)amine and corresponding amines following the procedure of example 21 or the alkylation/acylation of 3-({[3,5-bis(trifluoromethyl)benzyl](2-methyl-2H-tetrazol-5-yl)amino}methyl)-5-trifluoromethylpyridin-2-yl](alkyl)amines following the procedure of example 22.

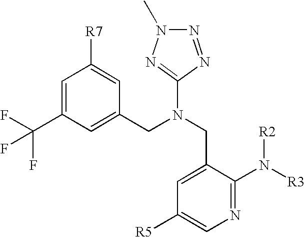

| | | | | | | ¹H-NMR (400 MHz, CDCl₃), |
|---|---|---|---|---|---|---|
| | | | | | MS or | δ (ppm) or HPLC/UPLC |
| No. | R₂ | R₃ | R₅ | R₇ | Rf value | Retention time |
| 23-1 | Et | n-Pr | CF₃ | CF₃ | 570 | 2.39 min (UPLC). |
| | | | | | [M + 1]⁺ | |
| 23-2 | Ac | n-Pr | CF₃ | CF₃ | Rf = 0.73 | 0.93 (t, 3H), 1.66 (ddd, 2H), 1.93 |
| | | | | | (Hex/EA = | (s, 3H), 4.02 (t, 2H), 4.19 (s, 3H), |
| | | | | | 1/1) | 4.65 (s, 2H), 4.77 (s, 2H), 7.54 (s, |
| | | | | | | 3H), 7.77 (s, 1H), 8.52 (d, 1H). |
| 23-3 | Et | n-Bu | CF₃ | CF₃ | 584 | 2.44 min (UPLC). |
| | | | | | [M + 1]⁺ | |
| 23-4 | Et | n-Pen | CF₃ | CF₃ | 598 | 2.59 min (UPLC). |
| | | | | | [M + 1]⁺ | |
| 23-4 | Et | 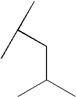 | CF₃ | CF₃ | 584[M + 1]⁺ | 2.43 min (UPLC). |
| 23-5 | Et | 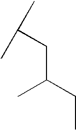 | CF₃ | CF₃ | 598[M + 1]⁺ | 2.49 min (UPLC). |
| 23-6 | Et | 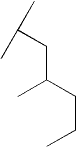 | CF₃ | CF₃ | 612[M + 1]⁺ | 2.53 min (UPLC). |
| 23-7 | Et | 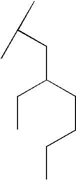 | CF₃ | CF₃ | 640[M + 1]⁺ | 2.61 min (UPLC). |
| 23-8 | Et | 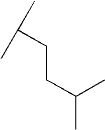 | CF₃ | CF₃ | 598[M + 1]⁺ | 2.49 min (UPLC). |
| 23-9 | Me | 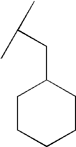 | CF₃ | CF₃ | 610[M + 1]⁺ | 0.71-0.85 (m, 2H), 1.28-1.55 (m,3H), 1.55-2.71 (m, 5H), 2.86 (s,3H), 3.03 (d, 2H), 4.23 (s, 3H),4.58 (s, 2H), 4.68 (s, 2H), 7.49 (d,1H), 7.57 (s, 2H), 7.75 (s, 1H),8.36 (s, 1H). |
| 23-10 | Et | 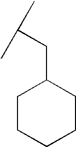 | CF₃ | CF₃ | 624[M + 1]⁺ | 0.70-0.83 (m, 2H), 1.05 (t, 3H),1.21-1.02 (m, 4H), 1.61-1.70 (m,5H), 3.08 (d, 2H), 3.17 (dd, 2H),4.22 (s, 3H), 4.60 (s, 2H), 4.68 (s,2H), 7.49 (d, 1H), 7.60 (s, 2H),7.75 (s, 1H), 8.39 (d, 1H). |
| 23-11 | n-Pr | 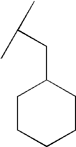 | CF₃ | CF₃ | 638[M + 1]⁺ | 0.74-0.88 (m, 7H) 1.07-1.18 (m,4H), 1.20-1.32 (m, 1H), 1.42-1.66(m, 4H), 1.61-1.70 (m, 5H), 3.08(d, 2H), 3.07-3.13 (m, 4H), 4.22(s, 3H), 4.60 (s, 2H), 4.68 (s, 2H),7.47 (s, 1H), 7.59 (s, 2H), 7.75 (s,1H), 8.38 (s, 1H). |
| 23-12 | | 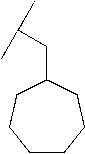 | CF₃ | CF₃ | 638[M + 1]⁺ | 1.03-1.10 (m, 2H), 1.05 (t, 3H),1.22-1.37 (m, 3H), 1.38-1.76 (m,8H), 3.08 (d, 2H), 3.15 (dd, 2H),4.22 (s, 3H), 4.60 (s, 2H), 4.69 (s,2H), 7.49 (d, 1H), 7.60 (s, 2H),7.75 (s, 1H), 8.40 (d, 1H). |
| 23-13 | Et | 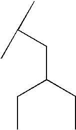 | CF₃ | CF₃ | 612[M + 1]⁺ | 2.55 min (UPLC). |
| 23-14 | Et | 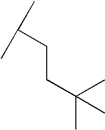 | CF₃ | CF₃ | 613[M + 1]⁺ | 2.53 min (UPLC). |
| 23-15 | Et | c-Pen | CF₃ | CF₃ | 596 | 0.92 (t, 3H), 1.40-1.60 (m, 4H), |
| | | | | | [M + 1]⁺ | 1.61-1.68 (m, 2H), 1.72-1.82 (m, |
| | | | | | | 2H), 3.23 (dd, 2H), 3.65-3.75 (m, |
| | | | | | | 1H), 4.22 (s, 3H), 4.60 (s, 2H), |
| | | | | | | 4.71 (s, 2H), 7.59 (d, 1H), 7.65 (s, |
| | | | | | | 2H), 7.77 (s, 1H), 8.45 (s, 1H). |
| 23-16 | Et | c-Hex | CF₃ | CF₃ | 610 | 2.53 min (UPLC). |
| | | | | | [M + 1]⁺ | |
| 23-17 | Et | c-Hep | CF₃ | CF₃ | 624 | 0.92 (t, 3H), 1.22-1.37 (m, 2H), |
| | | | | | [M + 1]⁺ | 1.40-1.60 (m, 4H), 1.43-1.58 (m, |
| | | | | | | 4H), 1.60-1.74 (m, 6H), 2.94-2.97 |
| | | | | | | (m, 1H), 3.31 (dd, 1H), 4.23 (s, |
| | | | | | | 3H), 4.58 (s, 2H), 4.66 (s, 2H), |
| | | | | | | 7.60 (d, 1H), 7.64 (s, 2H), 7.76 (s, |
| | | | | | | 1H), 8.43 (s, 1H). |
| 23-18 | Et | 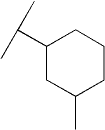 | CF₃ | CF₃ | 624[M + 1]⁺ | 2.53 min (UPLC). |
| 23-19 | Et | 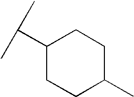 | CF₃ | CF₃ | 624[M + 1]⁺ | 2.54 min (UPLC). |
| 23-20 | Et | 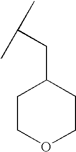 | CF₃ | CF₃ | 626[M + 1]⁺ | 2.32 min (UPLC). |
| 23-21 | Et | 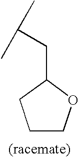 | CF₃ | CF₃ | 612[M + 1]⁺ | 2.31 min (UPLC). |
| 23-22 | Et | 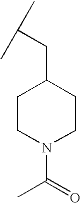 | CF₃ | CF₃ | 667[M + 1]⁺ | 0.80-1.10 (m, 2H), 1.07 (t, 3H),1.65-1.70 (m, 2H), 1.72-1.85 (m,1H), 2.05 (s, 3H), 2.44 (ddd, 1H),2.91 (ddd, 1H), 3.10-3.25 (m,4H), 3.70-3.75 (m, 1H), 4.22 (s,3H), 4.52-4.72 (m, 5H), 7.50 (dd,1H), 7.62 (s, 2H), 7.78 (s, 1H),8.40 (dd, 1H). |
| 23-23 | Et | 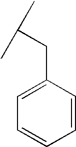 | CF₃ | CF₃ | 618[M + 1]⁺ | 2.43 min (UPLC). |
| 23-24 | Et | 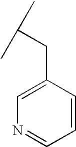 | CF₃ | CF₃ | 619[M + 1]⁺ | 1.11 (t, 3H), 3.30 (q, 2H), 4.20 (s,3H), 4.58 (s, 2H), 4.63 (s, 2H),4.74 (s, 2H), 7.09 (t, 1H), 7.17 (d,1H), 7.49 (t, 1H), 7.54 (s, 1H),7.59 (s, 1H), 7.75 (s, 1H), 8.38 (s,1H), 8.45 (d, 1H). |
| 23-25TAJ955 | Et | 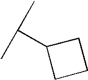 | CF₃ | CF₃ | 582[M + 1]⁺ | 2.41 min (UPLC) |
| 23-26TAJ956 | Et | 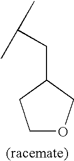 | CF₃ | CF₃ | 612[M + 1]⁺ | 2.28 min (UPLC) |
| 23-27TAJ957 | Et | 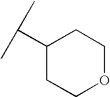 | CF₃ | CF₃ | 612[M + 1]⁺ | 2.33 min (UPLC) |
| 23-28TAK180 | Et | 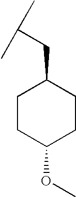 | CF₃ | CF₃ | 654[M + 1]⁺ | 2.40 min (UPLC) |
| 23-29 | Et | 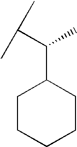 | CF₃ | CF₃ | Rf = 0.31(Hex/EA =5/1) | 0.39-0.53 (m, 1H), 0.72-0.84 (m,1H), 0.95 (t. 3H), 1.00-1.11 (m,1H), 1.08 (d. 3H), 1.12-1.24 (m,1H), 1.42-1.75 (m, 7H), 2.81-2.88(m, 1H), 3.17-3.25 (m, 1H), 3.34-3.43 (m, 1H), 4.24 (s, 3H), 4.45(d, 1H), 4.55 (q, 2H), 4.80 (d, 1H),7.53 (d, 1H), 7.60 (s, 2H), 7.77 (s,1H), 8.40 (s. 1H). |

### Example 24

The following compounds are prepared from [3,5-bis(trifluoromethyl)benzyl](2-chloro-5-trifluoromethylpyridin-3-ylmethyl)(2-methyl-2H-tetrazol-5-yl)amine and corresponding amines following the procedure of example 21.

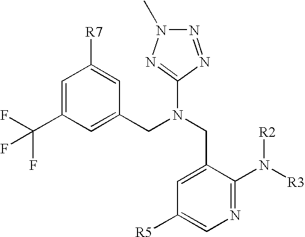

| | | | | | ¹H-NMR (400 MHz, CDCl₃), δ (ppm) |
|---|---|---|---|---|---|
| No. | NR₂R₃ | R₅ | R₇ | MS | or HPLC/UPLC Retention time |
| 24-1 | 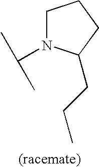 | CF₃ | CF₃ | 596[M + 1]⁺ | 0.89 (t, 3H), 1.15-1.34 (m, 4H),1.59-1.76 (m, 3H), 1.87-1.91 (m,1H), 2.07-2.13 (m, 1H), 3.20-3.24(m, 1H), 3.54-3.61 (m, 1H), 4.22 (s,3H), 4.23-4.34 (m, 1H), 4.46 (d,2H), 4.73 (d, 1H), 4.86 (d, 1H),7.42 (s, 1H), 7.58 (s, 2H), 7.75 (s,1H), 8.32 (s, 1H). |
| 24-2 | 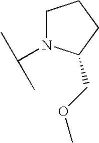 | CF₃ | CF₃ | 598[M + 1]⁺ | 1.72-1.98 (m, 3H), 2.05-2.27 (m,1H), 3.29 (s, 3H), 3.24-3.32 (m,2H), 3.47-3.50 (dd, 1H), 3.58-3.64(m, 1H), 4.22 (s, 3H), 4.49 (d, 1H),4.53 (d, 1H), 4.58-4.61 (m, 1H),4.23-4.34 (m, 1H), 4.46 (d, 2H),4.71 (d, 1H), 4.91 (d, 1H), 7.46 (d,1H), 7.61 (s, 2H), 7.75 (s, 1H),8.32 (s, 1H). |
| 24-3 | 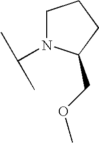 | CF₃ | CF₃ | 598[M + 1]⁺ | 1.72-1.98 (m, 3H), 2.05-2.27 (m,1H), 3.29 (s, 3H), 3.24-3.32 (m,2H), 3.47-3.50 (dd, 1H), 3.58-3.64(m, 1H), 4.22 (s, 3H), 4.49 (d, 1H),4.53 (d, 1H), 4.58-4.61 (m, 1H),4.23-4.34 (m, 1H), 4.46 (d, 2H),4.71 (d, 1H), 4.91 (d, 1H), 7.46 (d,1H), 7.61 (s, 2H), 7.75 (s, 1H),8.32 (s, 1H). |
| 24-4 | 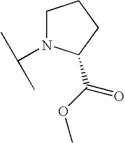 | CF₃ | CF₃ | 612[M + 1]⁺ | 1.89-2.01 (m, 2H), 2.02-2.13 (m,1H), 2.22-2.33 (m, 1H), 3.52-3.58(m, 1H), 3.71 (s, 3H), 3.71-3.80 (m,1H), 4.23 (s, 3H), 4.58-4.70 (m,3H), 4.70 (t, 1H), 5.00 (d, 1H), 7.43(d, 1H), 7.63 (s, 2H), 7.74 (s, 1H),8.22 (d, 1H). |
| 24-5 | 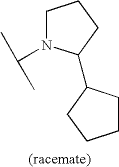 | CF₃ | CF₃ | 622[M + 1]⁺ | 2.42 min (UPLC). |
| 24-6 | 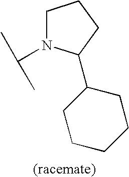 | CF₃ | CF₃ | 638[M + 1]⁺ | 0.90-1.25 (m, 5H), 1.43-1.46 (m,1H), 1.62-1.76 (m, 7H), 1.86-1.96(m, 1H), 1.99-2.07 (m, 1H), 3.16-3.21 (m, 1H), 3.44-3.53 (m, 1H),4.21 (s, 3H), 4.43-4.49 (m, 1H),4.45 (d, 1H), 4.57 (d, 1H), 4.69 (d,1H), 4.89 (d, 1H), 7.38 (d, 1H),7.60 (s, 2H), 7.76 (s, 1H), 8.31 (s,1H). |
| 24-7 | 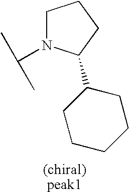 | CF₃ | CF₃ | — | 5.30 min (Condition B) |
| 24-8 | 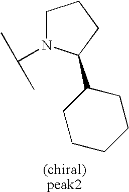 | CF₃ | CF₃ | — | 6.10 min (Condition B) |
| 24-9 | 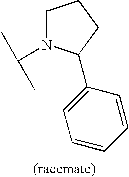 | CF₃ | CF₃ | 630[M + 1]⁺ | 1.83-2.00 (m, 3H), 3.58-3.64 (m,1H), 2.34-2.40 (m, 1H), 3.50-3.55(m, 1H), 3.88-3.94 (m, 1H), 4.22 (s,3H), 4.52 (dd, 2H), 4.61 (d, 1H),4.74 (d, 1H), 5.34 (t, 1H), 7.12-7.22(m, 5H), 7.30 (dd, 1H), 7.48 (s,2H), 7.75 (s, 1H), 8.24 (s, 1H). |
| 24-10 | 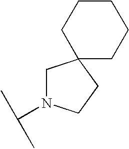 | CF₃ | CF₃ | 622[M + 1]⁺ | 2.26 min (UPLC). |
| 24-11 | 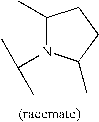 | CF₃ | CF₃ | 582[M + 1]⁺ | 1.16 (s, 3H), 1.17 (s, 3H), 1.64-1.70 (m, 2H), 1.92-1.99 (m, 2H),4.22 (s, 2H), 4.52 (dd, 2H), 4.05-4.09 (m, 1H), 4.22 (s, 3H), 4.63 (s,2H), 4.75 (s, 2H), 7.45 (d, 1H),7.57 (s, 2H), 7.74 (s, 1H), 8.33 (s,1H). |
| 24-12 | 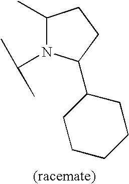 | CF₃ | CF₃ | 650[M + 1]⁺ | 0.86-1.19 (m, 5H), 1.19 (d, 3H),1.34-1.41 (m, 1H), 1.62-1.76 (m,8H), 1.55-1.81 (m, 1H), 1.99-1.84(m, 1H), 2.00-2.06 (m, 1H), 3.50-3.53 (m, 1H), 4.21 (s, 3H), 4.43-4.49 (m, 1H), 4.54 (d, 1H), 4.57 (d,1H), 4.64 (q, 1H), 4.80 (d, 1H),4.94 (d, 1H), 7.44 (d, 1H), 7.64 (s,2H), 7.77 (s, 1H), 8.33 (s, 1H). |
| 24-13 | 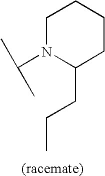 | CF₃ | CF₃ | 610[M + 1]⁺ | 0.77 (t, 3H), 1.03-1.14 (m, 3H),1.35-1.55 (m, 3H), 1.56-1.77 (m,2H), 3.11-3.14 (m, 2H), 3.39-3.46(m, 1H), 4.23 (s, 3H), 4.51 (dd,2H), 4.67 (d, 1H), 4.80 (d, 1H),7.57 (s, 1H), 7.61 (s, 2H), 7.76 (s,1H), 8.42 (s, 1H). |
| 24-14 | 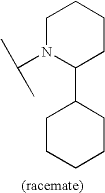 | CF₃ | CF₃ | 650[M + 1]⁺ | 0.40-0.51 (m, 1H), 0.75-0.88 (m,1H), 0.97-1.19 (m, 3H), 1.34-1.72(m, 10H), 1.77-1.93 (m, 2H), 3.08-3.20 (m, 1H), 3.29-3.39 (m, 2H),4.23 (s, 3H), 4.33 (d, 1H), 4.45 (d,1H), 4.67 (d, 1H), 4.94 (d, 1H),7.46 (s, 1H), 7.58 (s, 2H), 7.75 (s,1H), 8.35 (d, 1H). |
| 24-15 | 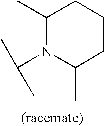 | CF₃ | CF₃ | 596[M + 1]⁺ | 0.61 (s, 3H), 0.63 (s, 3H), 1.24-1.38 (m, 2H), 1.68-1.82 (m, 4H),3.01-3.13 (m, 2H), 4.19 (s, 3H),4.78 (s, 2H), 4.89 (s, 2H), 7.63 (s,1H), 7.73 (s, 2H), 7.78 (s, 1H),8.67 (s, 1H). |
| 24-16 | 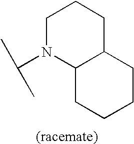 | CF₃ | CF₃ | 622[M + 1]⁺ | 0.66-0.77 (m, 1H), 1.07-1.37 (m,6H), 1.52-1.70 (m, 6H), 2.62-2.68(m, 1H), 2.77-2.80 (m, 1H), 2.84-2.90 (m, 1H), 4.21 (s, 3H), 4.66 (d,2H), 4.78-4.82 (m, 3H), 7.71-7.73(m, 3H), 7.78 (s, 1H), 8.59 (d, 1H). |
| 24-17 | 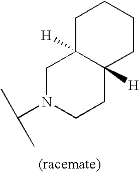 | CF₃ | CF₃ | 622[M + 1]⁺ | 0.93-1.31 (m, 8H), 1.52-1.66 (m,2H), 1.72-1.75 (m, 2H), 2.51 (dd,1H), 2.83 (ddd, 1H), 3.21 (m, 1H),3.35 (m, 1H), 4.23 (s, 3H), 4.54 (d,1H), 4.61 (d, 1H), 4.62 (d, 1H),4.71 (d, 1H), 7.58 (s, 2H), 7.60 (d,1H), 7.76 (s, 1H), 8.41 (d, 1H). |
| 24-18 | 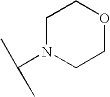 | CF₃ | CF₃ | 570[M + 1]⁺ | 3.15 (t, 4H), 3.74 (t, 4H), 4.23 (s,3H), 4.62 (s, 2H), 4.69 (s, 2H),7.59 (s, 2H), 7.62 (d, 1H), 7.76 (s,1H), 8.44 (d, 1H). |
| 24-19 | 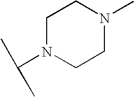 | CF₃ | CF₃ | 583[M + 1]⁺ | 2.31 (s, 3H), 2.39-2.41 (m, 4H),3.18 (t, 4H), 4.23 (s, 3H), 4.60 (s,2H), 4.67 (s, 2H), 7.59 (s, 2H),7.61 (d, 1H), 7.76 (s, 1H), 8.42 (s,1H). |
| 24-20 | 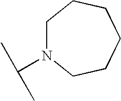 | CF₃ | CF₃ | 582[M + 1]⁺ | 149-1.60 (m, 4H), 1.69-1.76 (m,4H), 3.43 (t, 4H), 4.23 (s, 3H), 4.57(s, 2H), 4.67 (s, 2H), 7.46 (d, 1H),7.54 (s, 2H), 7.74 (s, 1H), 8.30 (s,1H). |
| 24-21TAJ948 | 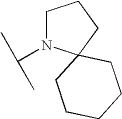 | CF₃ | CF₃ | 622[M + 1]⁺ | 2.52 min (UPLC) |
| 24-22TAK169 | 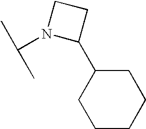 | CF₃ | CF₃ | 622[M + 1]⁺ | 2.42 min (UPLC) |

### Example 25

### Synthesis of [3-({[3,5-bis(trifluoromethyl)benzyl](2-methyl-2H-tetrazol-5-yl)amino}methyl)5-trifluoromethylpyridin-2-yl][(1,1-dioxohexahydro-2H-thiopyran-4-yl)methyl]ethylamine

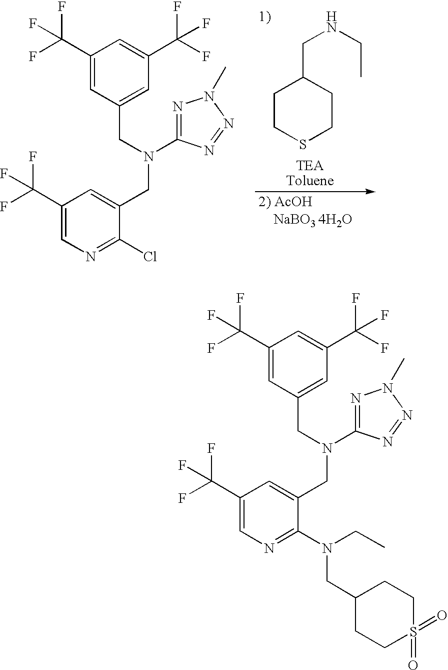

A suspension of [3,5-bis(trifluoromethyl)benzyl](2-chloro-5-trifluoromethylpyridin-3-ylmethyl)(2-methyl-2H-tetrazol-5-yl)amine (100 mg, 0.19 mmol), ethyl(tetrahydro-thiopyran-4-ylmethyl)amine (153 mg, 0.96 mmol), triethylamine (268 μL, 1.93 mmol) in toluene (1 mL) is stirred at 150° C. for 1 day in sealed tube. The reaction mixture is cooled to room temperature, and diluted with water and dichloromethane. The organic layer is filtered through phase separator and concentrated. The residue is dissolved in AcOH (5 mL) and sodium perborate tetrahydride (148 mg, 0.965 mmol) is added to the solution. After stirring at 55° C. for 15 hours, AcOH is removed by evaporation. Water is added to the residue and the mixture is extracted with dichloromethane. The organic layer is washed with brine, dried over magnesium sulfate and concentrated in vacuo. The crude product is purified by reverse-phase HPLC to give [3-({[3,5-bis(trifluoromethyl)benzyl](2-methyl-2H-tetrazol-5-yl)amino}methyl)-5-trifluoromethylpyridin-2-yl][(1,1-dioxohexahydro-2H-thiopyran-4-yl)methyl]ethylamine (12 mg, 9%).

ESI-MS m/z: 674 [M+1]⁺. UPLC retention time: 2.23 min.

### Example 26

### Synthesis of [3-({[3,5-bis(trifluoromethyl)benzyl](5-bromopyrimidin-2-yl)amino}methyl)-5-trifluoromethylpyridine-2-yl](cyclopentylmethyl)ethylamine

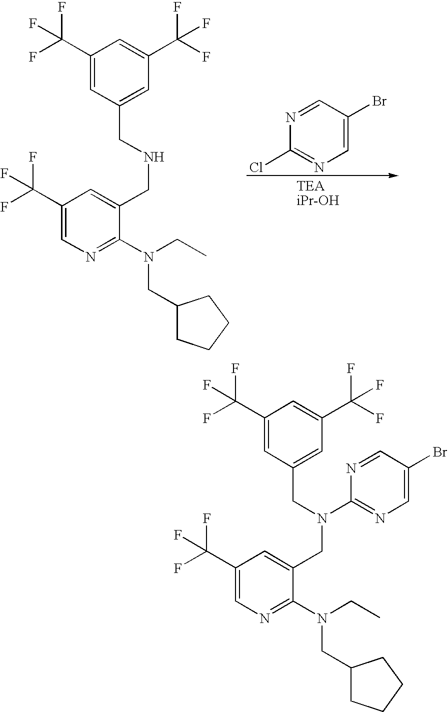

A suspension of [3-({[3,5-bis(trifluoromethyl)benzyl]amino}methyl)-5-trifluoromethylpyridine-2-yl](cyclopentylmethyl)ethylamine (959 mg, 1.8 mmol), 5-bromo-2-chloropyrimidine (854 mg, 4.4 mmol), and triethylamine (607 μL, 4.4 mmol) in i-PrOH (8.5 mL) is irradiated with microwaves at 200° C. for 40 min. After cooling to room temperature, the reaction mixture is diluted with water and EtOAc. The organic layer is washed with water, brine, dried over sodium sulfate, filtered, and concentrated. The crude product is purified by silica gel column chromatography to give [3-({[3,5-bis(trifluoromethyl)benzyl](5-bromopyrimidin-2-yl)amino}methyl)-5-trifluoromethylpyridine-2-yl](cyclopentylmethyl)ethylamine (541 mg).

¹H-NMR (400 MHz, CDCl₃), δ (ppm): 1.03-1.10 (m, 2H), 1.07 (t, 3H), 1.44-1.58 (m, 6H), 2.05-2.15 (m, 1H), 3.15-3.21 (m, 4H), 4.77 (s, 2H), 4.84 (s, 2H), 7.38 (d, 1H), 7.63 (s, 2H), 7.76 (s, 1H), 8.41 (s, 3H).

ESI-MS m/z: 684, 686 [M+1]⁺

### Example 27

### Synthesis of [3-({[3,5-bis(trifluoromethyl)benzyl][5-(4-methylpiperazine-1-yl)pyrimidin-2-yl]amino}methyl)-5-trifluoromethylpyridin-2-yl](cyclopentylmethyl)ethylamine (TAK166)

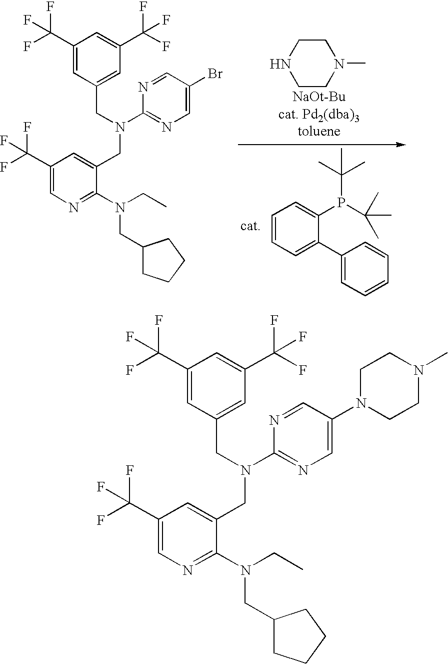

A suspension of [3-({[3,5-bis(trifluoromethyl)benzyl](5-bromopyrimidin-2-yl)amino}methyl)-5-trifluoromethylpyridine-2-yl](cyclopentylmethyl)ethylamine (99 mg, 0.15 mmol), 1-methylpiperazine (23 mg, 0.23 mmol), NaOt-Bu (21 mg, 0.22 mmol), Pd₂(dba)₃ (15 mg, 0.02 mmol), and 2-(di-t-butylphosphino)biphenyl (4 mg, 0.01 mmol) in toluene (1.5 mL) is stirred and refluxed for 4 hours. The reaction mixture is cooled to room temperature, and then diluted with water and ethyl acetate. The organic layer is washed with brine, dried over magnesium sulfate, filtered and concentrated. The crude product is purified by silica gel column chromatography to give [3-({[3,5-bis(trifluoromethyl)benzyl][5-(4-methylpiperazine-1-yl)pyrimidin-2-yl]amino}methyl)-5-trifluoromethylpyridin-2-yl](cyclopentylmethyl)ethylamine (48 mg).

¹H-NMR (400 MHz, CDCl₃), δ (ppm): 1.01-1.10 (m, 2H), 1.05 (t, 3H), 1.44-1.58 (m, 6H), 2.04-2.14 (m, 1H), 2.37 (s, 3H), 2.60-2.62 (m, 4H), 3.12-3.14 (m, 4H), 3.15-3.20 (m, 4H), 4.76 (s, 2H), 4.82 (s, 2H), 7.44 (d, 1H), 7.63 (s, 2H), 7.73 (s, 1H), 8.18 (s, 2H), 8.39 (d, 1H). ESI-MS m/z: 704 [M+1]⁺

### Example 28

The following compounds are prepared from [3-({[3,5-bis(trifluoromethyl)benzyl](5-bromopyrimidin-2-yl)amino}methyl)-5-trifluoromethylpyridine-2-yl](cyclopentylmethyl)ethylamine and corresponding amines following the procedure of example 27.

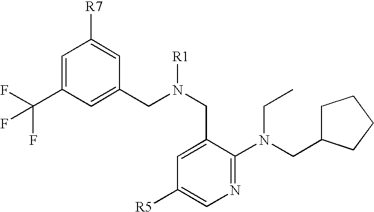

| | | | | | ^{H-NMR (400 MHz, CDCl}₃), δ (ppm) |
|---|---|---|---|---|---|
| No. | R₁ | R₅ | R₇ | MS | or HPLC/UPLC Retention time |
| 28-1TAJ987 | 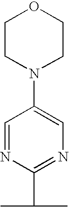 | CF₃ | CF₃ | 691[M + 1]⁺ | 1.01-1.10 (m, 2H), 1.06 (t, 3H),1.44-1.58 (m, 6H), 2.04-2.14 (m,1H), 3.06-3.09 (m, 4H), 3.14-3.21(m, 4H), 3.87-3.90 (m, 4H), 4.77 (s,2H), 4.83 (s, 2H), 7.44 (d, 1H),7.64 (s, 2H), 7.74 (s, 1H), 8.16 (s,2H), 8.39 (d, 1H). |
| 28-2TAK092 | 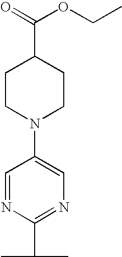 | CF₃ | CF₃ | 761[M + 1]⁺ | 1.01-1.09 (m, 2H), 1.06 (t, 3H),1.28 (t, 3H), 1.43-1.58 (m, 6H),1.87-1.97 (m, 2H), 2.04-2.14 (m,3H), 2.40-2.45 (m, 1H), 2.75-2.82(m, 2H), 3.15-3.20 (m, 4H), 3.40-3.45 (m, 2H), 4.17 (q, 2H), 4.76 (s,2H), 4.82 (s, 2H), 7.44 (d, 1H),7.63 (s, 2H), 7.73 (s, 1H), 8.17 (s,2H), 8.39 (d, 1H). |
| 28-3TAK179 | 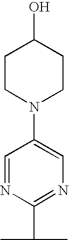 | CF₃ | CF₃ | 705[M + 1]⁺ | 1.01-1.10 (m, 2H), 1.06 (t, 3H),1.40-1.58 (m, 6H), 1.71-1.80 (m,2H), 2.02-2.15 (m, 3H), 2.86-2.93(m, 2H), 3.15-3.21 (m, 4H), 3.34-3.40 (m, 2H), 3.83-3.91 (m, 1H),4.76 (s, 2H), 4.82 (s, 2H), 7.44 (d,1H), 7.64 (s, 2H), 7.73 (s, 1H),8.19 (s, 2H), 8.39 (d, 1H). |

### Example 29

[3,5-bis(trifluoromethyl)benzyl)][2-((R)-2-cyclohexylpyrrolidin-1-yl)-5-trifluoromethylpyridin-3-ylmethyl](5-morpholin-4-yl-pyrimidin-2-yl)amine is prepared by following the procedure of example 27.

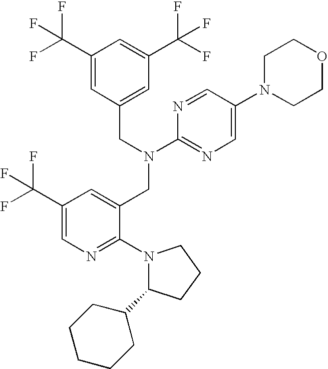

¹H-NMR (400 MHz, CDCl₃), δ (ppm): 0.87-2.09 (m, 15H), 3.07 (t, 4H), 3.22-3.29 (m, 1H), 3.43-3.55 (m, 1H), 3.88 (t, 4H), 4.32 (d, 1H), 4.44-4.54 (m, 1H), 4.57 (d, 1H), 5.03 (d, 1H), 5.27 (d, 1H), 7.30 (s, 1H), 7.64 (s, 2H), 7.74 (s, 1H), 8.14 (s, 2H), 8.29 (s, 1H).

Rf value=0.20 (Hexane/AcOEt=1/1)

### Example 30

### Synthesis of 1-[2-([3,5-bis(trifluoromethyl)benzyl]{[2-(cyclopentylmethyl)ethylamino-5-trifluoromethylpyridine-5-yl]methyl}amino)pyrimidine-4-yl]-piperidine-4-carboxylic acid (TAK093)

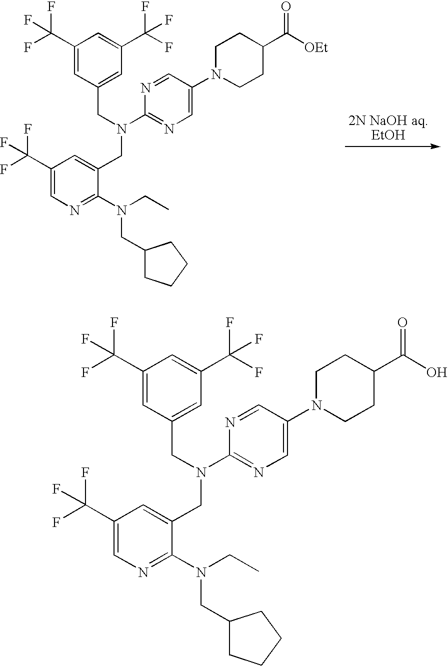

To a solution of 1-[2-([3,5-bis(trifluoromethyl)benzyl]{[2-(cyclopentylmethyl)ethylamino-5-trifluoromethylpyridine-5-yl]methyl}amino)pyrimidine-4-yl]-piperidine-4-carboxylic acid ethyl ester (31 mg, 0.040 mmol) in EtOH (1.0 mL) is added 2N NaOH (80 μL) and the mixture is stirred at room temperature for 19 hours. The mixture is diluted with 1N HCl and ethyl acetate, and the organic layer is washed with brine, dried over magnesium sulfate, filtered and concentrated to give 1-[2-([3,5-bis(trifluoromethyl)benzyl]{[2-(cyclopentylmethyl)ethylamino-5-trifluoromethylpyridine-5-yl]methyl}amino)pyrimidine-4-yl]-piperidine-4-carboxylic acid (29 mg).

¹H-NMR (400 MHz, CDCl₃), δ (ppm): 1.01-1.10 (m, 2H), 1.06 (t, 3H), 1.40-1.58 (m, 6H), 1.90-2.03 (m, 2H), 2.05-2.15 (m, 3H), 2.43-2.55 (m, 1H), 2.78-2.87 (m, 2H), 3.15-3.22 (m, 4H), 3.40-3.47 (m, 2H), 4.77 (s, 2H), 4.83 (s, 2H), 7.44 (d, 1H), 7.63 (s, 2H), 7.73 (s, 1H), 8.19 (s, 2H), 8.40 (d, 1H).

ESI-MS m/z: 733 [M+1]⁺

### Example 31

### Synthesis of trans-(4-{(R)-1-[3-({[3,5-bis(trifluoromethyl)benzyl](2-methyl-2H-tetrazol-5-yl)amino}methyl)-5-trifluoromethylpyridine-2-yl]pyrrolidin-2-yl}cyclohexyl)acetic acid (TAK472)

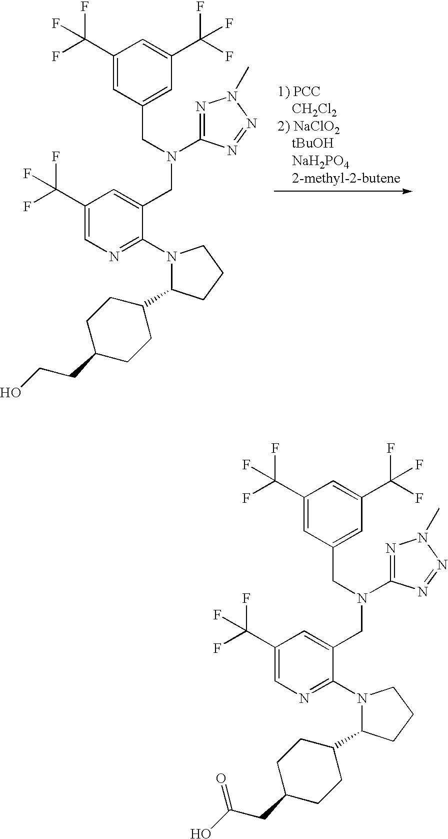

### Step 1:

A suspension of trans-(4-{(R)-1-[3-({[3,5-bis(trifluoromethyl)benzyl](2-methyl-2H-tetrazol-5-yl)amino}methyl)-5-trifluoromethylpyridine-2-yl]pyrrolidin-2-yl}cyclohexyl)ethanol (61 mg, 0.090 mmol) and PCC (97 mg, 0.045 mmol) in CH₂Cl₂ (0.5 mL) is stirred at room temperature for 3 hours. The reaction mixture is quenched by addition of ethanol, filtered and concentrated. The crude product is purified by silica gel column chromatography to give trans-(4-{(R)-1-[3-({[3,5-bis(trifluoromethyl)benzyl](2-methyl-2H-tetrazol-5-yl)amino}methyl)-5-trifluoromethylpyridine-2-yl]pyrrolidin-2-yl}cyclohexyl)acetoaldehyde (11.8 mg).

### Step 2:

A mixture of NaClO₂ (6.3 mg, 0.070 mmol) and 0.42 M NaH₂PO₄ aqueous solution (125 μL, 0.63 mmol) is added dropwise to a solution of trans-(4-{(R)-1-[3-({[3,5-bis(trifluoromethyl)benzyl](2-methyl-2H-tetrazol-5-yl)amino}methyl)-5-trifluoromethylpyridine-2-yl]pyrrolidin-2-yl}cyclohexyl)acetoaldehyde (11.8 mg, 0.015 mmol) and 2-methyl-2-butene (0.30 mL) in t-BuOH (0.30 mL), and the resulting mixture is stirred at room temperature for 7 hours. The reaction mixture is quenched by addition of sat. NH₄Cl aq. and extracted with CH₂Cl₂. The organic layer is filtered through phase separator and concentrated to give trans-(4-{(R)-1-[3-({[3,5-bis(trifluoromethyl)benzyl](2-methyl-2H-tetrazol-5-yl)amino}methyl)-5-trifluoromethylpyridine-2-yl]pyrrolidin-2-yl}cyclohexyl)acetic acid (8.7 mg).

Rf value=0.10 (Hexane/AcOEt=3/1)

¹H-NMR (400 MHz, CDCl₃), δ (ppm): 0.76-1.96 (m, 13H), 2.19 (d, 1H), 3.17-3.23 (m, 1H), 3.29 (s, 2H), 3.46-3.57 (m, 1H), 4.20 (s, 3H), 4.46 (d, 1H), 4.42-4.53 (m, 1H), 4.57 (d, 1H), 4.67 (d, 1H), 4.88 (d, 1H), 7.38 (s, 1H), 7.60 (s, 2H), 7.76 (s, 1H), 8.31 (s, 1H).

Rf value=0.17 (Hexane/AcOEt=5/1)

### Example 32

### Synthesis of trans-4-{(R)-1-[(3-({[3,5-bis(trifluoromethyl)benzyl](5-morpholin-4-yl-pyrimidin-2-yl)amino}methyl)-5-trifluoromethylpyridin-2-yl]pyrrolidin-2-yl}cyclohexanecarboxylic acid (TAK779)

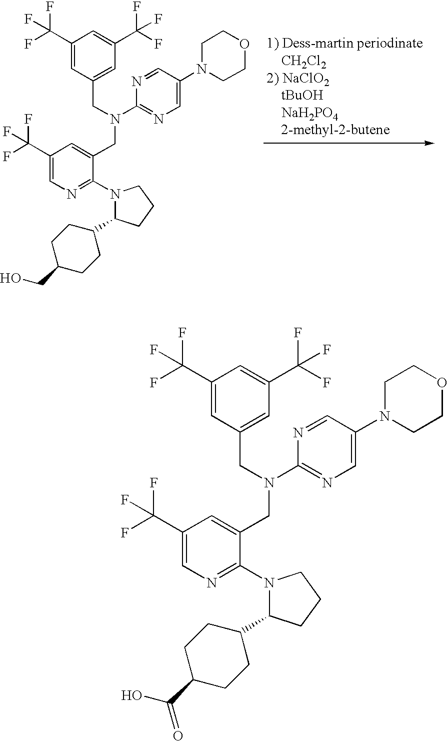

### Step 1:

A suspension of trans-(4-{(R)-1-[3-({[3,5-bis(trifluoromethyl)benzyl](5-morpholin-4-yl-pyrimidin-2-yl)amino}methyl)-5-trifluoromethylpyridin-2-yl]pyrrolidin-2-yl}cyclohexyl)methanol (304 mg, 0.41 mmol) and Dess-Martin periodinane (190 mg, 0.45 mmol) in CH₂Cl₂ (3.0 mL) is stirred at room temperature for 1.5 hours. Dess-Martin periodinane (90 mg, 0.22 mmol) is added to the reaction mixture and the resulting solution is further stirred at room temperature for 1 hours. The reaction mixture is quenched by addition of 1N NaOH and extracted with CH₂Cl₂ twice. The combined organic layers are washed with brine, dried over magnesium sulfate, filtered and concentrated. The crude product is purified by silica gel column chromatography to give trans-4-{(R)-1-[3-({[3,5-bis(trifluoromethyl)benzyl](5-morpholin-4-yl-pyrimidin-2-yl)amino}methyl)-5-trifluoromethylpyridin-2-yl]pyrrolidin-2-yl}cyclohexanecarbardehyde (228 mg).

### Step 2:

A mixture of NaClO₂ (130 mg, 1.4 mmol) and 0.36 M NaH₂PO₄ aqueous solution (3.0 mL, 0.63 mmol) is added dropwise to a solution of trans-4-{(R)-1-[3-({[3,5-bis(trifluoromethyl)benzyl](5-morpholin-4-yl-pyrimidin-2-yl)amino}methyl)-5-trifluoromethylpyridin-2-yl]pyrrolidin-2-yl}cyclohexanecarbardehyde (228 mg, 0.31 mmol) and 2-methyl-2-butene (3.0 mL) in t-BuOH (3.0 mL), and the resulting mixture is stirred at room temperature for 1.5 hours. The reaction mixture is quenched by addition of sat. NH₄Cl aq. and extracted with CH₂Cl₂. The organic layer is filtered through phase separator and concentrated. The crude product is purified by silica gel column chromatography to give trans-4-{(R)-1-[3-({[3,5-bis(trifluoromethyl)benzyl](5-morpholin-4-yl-pyrimidin-2-yl)amino}methyl)-5-trifluoromethylpyridin-2-yl]pyrrolidin-2-yl}cyclohexanecarboxylic acid.

¹H-NMR (400 MHz, CDCl₃), δ (ppm): 1.10-2.28 (m, 14H), 3.07 (t, 4H), 3.18-3.27 (m, 1H), 3.46-3.56 (m, 1H), 3.87 (t, 4H), 4.37 (d, 1H), 4.49-4.57 (m, 1H), 4.61 (d, 1H), 5.03 (d, 1H), 5.22 (d, 1H), 7.32 (s, 1H), 7.64 (s, 2H), 7.75 (s, 1H), 8.14 (s, 2H), 8.29 (s, 1H).

Rf value=0.20 (Hexane/AcOEt=1/1)

### General UPLC Condition

Column: Waters ACQUITY UPLC BEH C18, 1.7 μM

Mobile phase: CH₃CN/H₂O (0.1% TFA)

### HPLC Condition B

Column: Chiralpak OD-H, 4.6×150 mm

Mobile phase: 1% IPA/Hexane

The starting materials can be prepared, for example, as follows:

### Example A

### Preparation of {2-[(cyclopentylmethyl)ethylamino]-5-trifluoromethylpyridin-3-yl}methanol

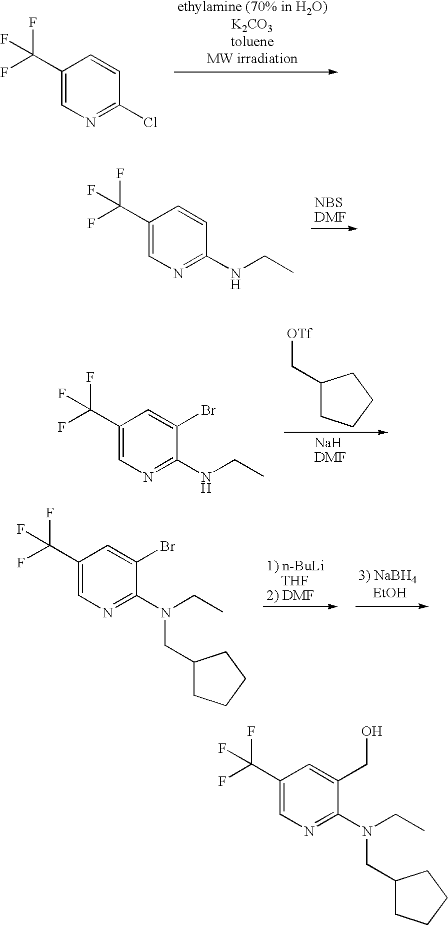

### Step 1:

A suspension of 2-chloro-5-trifluoromethylpyridine (2.16 g, 0.012 mmol), 70% ethylamine in water (3 mL), potassium carbonate (3.29 g, 0.023 mmol) in toluene is irradiated in a microwave reactor for 30 min. After adding water, the mixture is extracted with ethyl acetate. The combined organic layer is washed with brine, dried over magnesium sulfate, filtrated and concentrated to give ethyl(5-trifluoromethyl-pyridin-2-yl)amine (1.88 g, 83% yield).

¹H-NMR (400 MHz, CDCl₃), δ (ppm): 1.28 (t, 3H), 3.32-3.39 (m, 2H), 4.82 (br, 1H), 6.38 (d, 1H), 7.58 (dd, 1H), 8.32 (d, 1H).

### Step 2:

A solution of ethyl(5-trifluoromethylpyridin-2-yl)amine (1.87 g, 9.8 mmol) in DMF (20 mL) is treated with N-bromosuccinimide (2.10 g, 11.8 mmol) for 2 hours at ambient temperature. After adding water, the mixture is extracted with ethyl acetate. The combined organic layer is washed with water (3 times) and brine, dried over magnesium sulfate, filtrated and concentrated in vacuo to give (3-bromo-5-trifluoromethyl-pyridin-2-yl)ethylamine (2.55 g, 95% yield).

¹H-NMR (400 MHz, CDCl₃), δ (ppm): 1.29 (t, 3H), 3.51-3.57 (m, 2H), 5.33 (br, 1H), 7.78 (d, 1H), 8.31 (d, 1H).

### Step 3:

To a solution of cyclopentylmethanol (0.40 g, 4.0 mmol) in dichloromethane, pyridine (0.35 g, 4.4 mmol) and trifluoromethansulfonic anhydride (0.90 mL, 4.2 mmol) are added at 0° C., successively, and the mixture is stirred at the same temperature for 1 hour. After adding water, the mixture is extracted with dichloromethane. The organic layer is washed with brine, dried over magnesium sulfate, filtrated and concentrated in vacuo to give crude cyclopenthylmethyl trifluoromethanesulfonate.

To a stirring solution of (3-bromo-5-trifluoromethylpyridin-2-yl)ethylamine (0.27 g, 1.0 mmol) in DMF, sodium hydride (0.080 g, 2.0 mmol) is added and the reaction mixture is stirred at room temperature over 25 min. A solution of the crude cyclopenthylmethyl trifluoromethane-sulfonate prepared above in DMF is added dropwise to the mixture, which is allowed to stir at ambient temperature for 30 min. After adding sat. sodium hydrogen carbonate solution, the mixture is extracted with ethyl acetate. The combined organic layer is washed with water and then brine, dried over magnesium sulfate, filtrated and concentrated in vacuo. The residue is purified by silica gel column chromatography (AcOEt/hexane=5/95 to 50/50) to give (3-bromo-5-trifluoromethylpyridin-2-yl)(cyclopentylmethyl)ethylamine (0.17 g, 48%).

¹H-NMR (400 MHz, CDCl₃), δ (ppm): 1.11-1.20 (m, 2H), 1.18 (t, 3H), 1.45-1.70 (m, 6H), 2.15-2.22 (m, 1H), 3.42 (d, 2H), 3.52 (q, 2H), 7.90 (d, 1H), 8.37 (d, 1H).

### Step 4:

A solution of (3-bromo-5-trifluoromethylpyridin-2-yl)(cyclopentylmethyl)ethylamine (0.17 g, 0.48 mmol) in THF is treated with n-butyl lithium (1.5M in hexane, 1.2 mL, 1.8 mmol) at −78° C. for 5 min. To the mixture, DMF (0.5 mL) is added. After stirring, sat. ammonium chloride solution and ethyl acetate are added and the mixture is warmed to room temperature. After extraction with ethyl acetate, the organic layer is washed with brine, dried over magnesium sulfate, filtrated and concentrated in vacuo to give crude 2-[(cyclopentyl methyl)ethylamino]-5-trifluoromethylpyridine-3-carbaldehyde.

To a mixture of crude 2-[(cyclopentyl methyl)ethylamino]-5-trifluoromethylpyridine-3-carbaldehyde obtained above in ethanol (1 mL), 30 mg (0.80 mmol) of sodium borohydride is added and the mixture is stirred for 5 hours at room temperature. After addition of sat. ammonium chloride, the mixture is extracted with ethyl acetate. The combined organic layer is washed with brine, dried over magnesium sulfate, filtrated and concentrated under reduced pressure. The residue is purified by silica gel column chromatography to give [2-[(cyclopentylmethyl)ethylamino]-5-trifluoromethylpyridin-3-yl]methanol (0.055 g, 2 steps 38%).

¹H-NMR (400 MHz, CDCl₃), δ (ppm): 1.08-1.16 (m, 2H), 1.11 (t, 3H), 1.45-1.70 (m, 6H), 2.05-2.14 (m, 1H), 3.04 (br, 1H), 3.24 (d, 2H), 3.27 (q, 2H), 4.71 (s, 2H), 7.83 (d, 1H), 8.46 (d, 1H).

### Example B

(3-bromo-5-trifluoromethylpyridin-2-yl)(cyclopentylmethyl)ethylamine can alternatively be prepared as follows

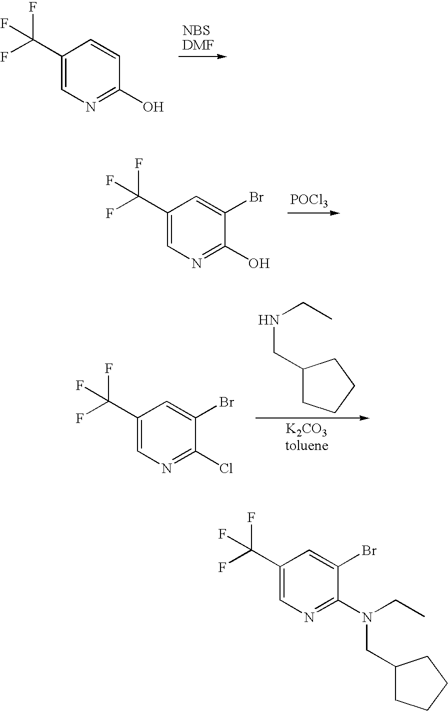

### Step 1:

N-bromosuccinimide (NBS, 39.00 g, 0.22 mol) is added portionwise to a solution of 5-(trifluoromethyl)pyridin-2-ol (30.00 g, 0.18 mol) in DMF (180 mL), and the resulting mixture is stirred for 2 hours. The mixture is poured into water (1200 mL) and the precipitate was collected by filtration. The crystal is dried in vacuo to give the product as a white solid (1st crystal: 28.10 g). The filtrate is extracted with EtOAc, and the organic layer is concentrated. The residue is poured into water and the precipitate is collected by filtration. The crystal is dried in vacuo to give 3-bromo-5-(trifluoromethyl)pyridin-2-ol (2nd crystal: 9.65 g, total: 37.75 g, 85% yield) as a yellow solid.

¹H-NMR (400 MHz, CDCl₃), δ (ppm): 7.86 (d, 1H), 8.02 (d, 1H), 13.17 (br, 1H).

### Step 2:

A mixture of 3-bromo-5-(trifluoromethyl)pyridin-2-ol (37.75 g, 0.16 mol) and phosphorus(III)oxychloride (POCl₃; 75 mL) is stirred at 10° C. for 5 hours. After cooling to room temperature, the mixture is poured into ice-water, and extracted with CH₂Cl₂ twice. The combined organic layer is washed with NaHCO₃ aq., brine, dried over MgSO₄, filtered and concentrated in vacuo. The crude mixture is purified by flash column chromatography to give 3-bromo-2-chloro-5-trifluoromethylpyridine (31.90 g, 79% yield) as a white solid.

¹H-NMR (400 MHz, CDCl₃), δ (ppm): 8.17 (m, 1H), 8.62 (d, 1H).

### Step 3:

A suspension of 3-bromo-2-chloro-5-trifluoromethylpyridine (1.00 g, 3.8 mmol), (cyclopenthylmethyl)ethylamine (0.63 g, 4.6 mmol), potassium carbonate (1.06 g, 7.7 mmol) in toluene is irradiated in a microwave reactor for 30 min. After adding water, the mixture is extracted with ethyl acetate. The combined organic layer is washed with brine, dried over magnesium sulfate, filtrated and concentrated to give (3-bromo-5-trifluoromethylpyridin-2-yl)(cyclopentylmethyl)ethylamine (1.32 g, 98%), which is used for the next reaction without further purification.

¹H-NMR (400 MHz, CDCl₃), δ (ppm): 1.11-1.20 (m, 2H), 1.18 (t, 3H), 1.45-1.70 (m, 6H), 2.15-2.22 (m, 1H), 3.42 (d, 2H), 3.52 (q, 2H), 7.90 (d, 1H), 8.37 (d, 1H).

### Example C

### Preparation of (4-{[ethyl-(3-hydroxymethyl-5-trifluoromethyl-pyridin-2-yl)amino]methyl}cyclohexyl)acetic acid ethyl ester

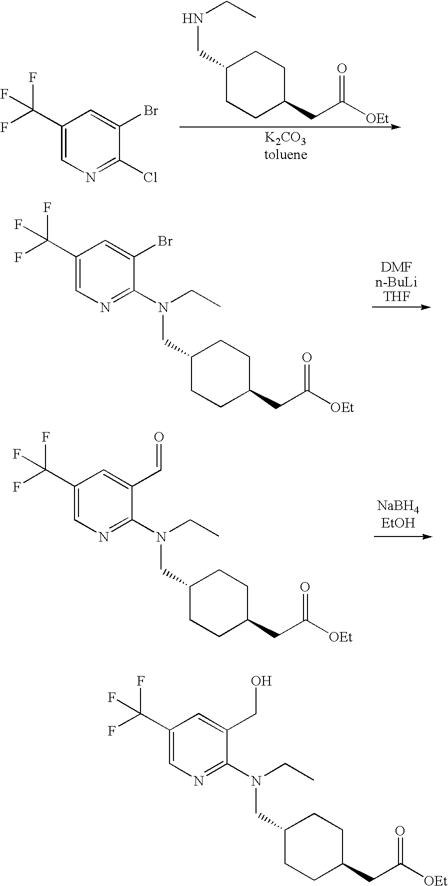

### Step 1:

A mixture of 3-bromo-2-chloro-5-trifluoromethylpyridine (12.5 g, 44 mmol), trans-[4-(ethylaminomethyl)cyclohexyl]acetic acid ethyl ester (10 g, 44 mmol), potassium carbonate (15.2 g. 0.11 mol) in toluene (88 mL) is stirred under reflux condition for 2 days. After cooling to room temperature, water and ethyl acetate are added and the mixture is extracted with ethyl acetate. The combined organic layer is washed with brine, dried over magnesium sulfate, filtered and concentrated. The residue is purified by silica gel column chromatography (hexane:AcOEt=95:5-1:1) to give trans-(4-{([(3-bromo-5-trifluoromethyl-pyridin-2-yl)ethylamino]methyl}cyclohexyl)acetic acid ethyl ester (13.0 g, 65% yield).

¹H-NMR (400 MHz, CDCl₃), δ (ppm): 0.88-0.95 (m, 4H), 1.18 (t, 3H), 1.25 (t, 3H), 1.48-1.53 (m, 1H), 1.65-1.79 (m, 5H), 2.15 (d, 2H), 3.34 (d, 2H), 3.50 (q, 2H), 4.11 (q, 2H), 7.89 (m, 1H), 8.36 (m, 1H).

### Step 2:

A solution of trans-(4-{[(3-bromo-5-trifluoromethylpyridin-2-yl)ethylamino]methyl}cyclohexyl)acetic acid ethyl ester (8.7 g, 19 mmol) and DMF (2.11 g, 29 mmol) in THF (60 mL) is cooled to −78° C. n-Butyl lithium solution (1.5 M in hexane, 14.1 mL, 21 mmol) is added dropwise over 12 min at −78-−68° C. to the mixture and the mixture is warmed to room temperature. After the mixture is stirred for 5 min at the same temperature, 1N HCl aq and ethyl acetate are added. The organic layer is washed with brine, dried over magnesium sulfate, filtered and concentrated in vacuo to give crude trans-(4-{[ethyl(3-formyl-5-trifluoromethylpyridin-2-yl)amino]methyl}cyclohexyl)acetic acid ethyl ester.

To a solution of crude trans-(4-{[ethyl(3-formyl-5-trifluoromethylpyridin-2-yl)amino]methyl}cyclohexyl)acetic acid ethyl ester in ethanol (60 mL), sodium borohydride (0.50 g, 13 mmol) is added and the mixture is stirred at ambient temperature for 12 hours. After addition of sat. ammonium chloride solution, water and ethyl acetate are added. After partition, the combined organic layer is washed with brine, dried over magnesium sulfate, filtered and concentrated in vacuo. The residue is purified by silica gel column chromatography (hexane:AcOEt=95:5-4:6) to afford trans-(4-{[ethyl(3-hydroxymethyl-5-trifluoromethylpyridin-2-yl)amino]methyl}cyclohexyl)acetic acid ethyl ester (3.34 g, 43% yield, 2 steps).

¹H-NMR (400 MHz, CDCl₃), δ (ppm): 0.88-0.95 (m, 4H), 1.10 (t, 3H), 1.24 (t, 3H), 1.45-1.55 (m, 1H), 1.65-1.79 (m, 5H), 2.15 (d, 2H), 2.66 (t, 1H), 3.18 (d, 2H), 3.25 (q, 2H), 4.10 (q, 2H), 4.69 (d, 2H), 7.86 (d, 1H), 8.43 (d, 1H).

### Example D

### Preparation of (4-{[(5-bromo-3-hydroxymethylpyridin-2-yl)ethylamino]methyl}cyclohexyl)acetic acid ethyl ester

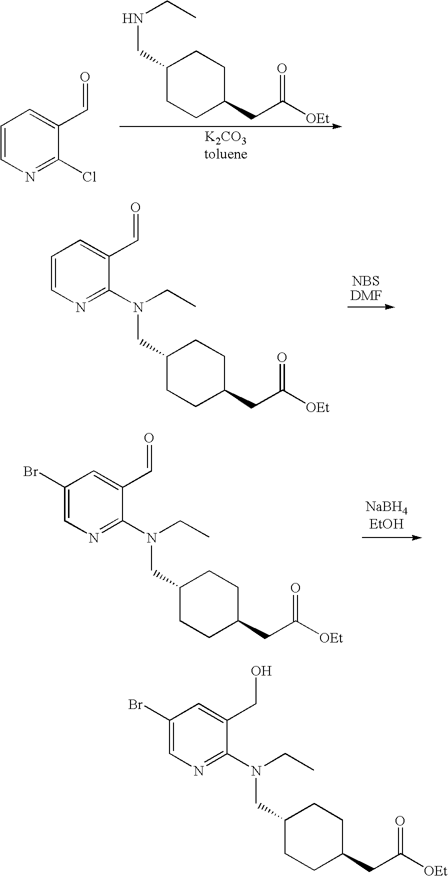

### Step 1:

To a solution of 2-chloro-pyridine-3-carbaldehyde (500 mg, 3.5 mmol) in toluenel (3 mL), trans-[4-(ethylaminomethyl)cyclohexyl]acetic acid ethyl ester (0.97 g, 4.3 mmol) and potassium carbonate (700 mg, 5.1 mmol) are added and the mixture is stirred at 120° C. for 12 hours. After cooling, ethyl acetate is added and the solution is washed with water, dried and concentrated in vacuo. The residue is purified by silica gel column chromatography (hexane: AcOEt=3:1) to give trans-(4-{[ethyl(3-formylpyridin-2-yl)amin]methyl}cyclohexyl-acetic acid ethyl ester as yellow oil (0.63 g, 54% yield).

¹H-NMR (400 MHz, CDCl₃), δ (ppm): 0.88-0.93 (m, 4H), 1.18 (t, 3H), 1.22-1.28 (m, 4H), 1.60-1.75 (m, 7H), 2.15 (d, 2H), 3.36 (d, 2H), 3.46 (q, 2H), 4.10 (q, 2H), 6.81 (dd, 1H), 7.96 (d, 1H), 8.32 (d, 1H), 9.98 (s, 1H).

### Step 2:

To a solution of trans-(4-{[ethyl-(3-formylpyridin-2-yl)amino]methyl}cyclohexyl)acetic acid ethyl ester (630 mg, 1.89 mmol) in DMF (6 mL), N-bromosuccinimide (401 mg, 2.3 mmol) is added. After stirring for 2 hours at ambient temperature, water is added. The mixture is extracted with ethyl acetate, washed with water, dried and concentrated under reduced pressure. The residue is purified by silica gel column chromatography (hexane:AcOEt=5:1) to give trans-(4-{[(5-bromo-3-formylpyridin-2-yl)ethylamino]methyl}cyclohexyl)acetic acid ethyl ester as pale yellow oil (0.58 g, 75% yield).

¹H-NMR (400 MHz, CDCl₃), δ (ppm): 0.85-0.99 (m, 4H), 1.18 (t, 3H), 1.22-1.28 (m, 4H), 1.60-1.75 (m, 7H), 2.15 (d, 2H), 3.34 (d, 2H), 3.44 (q, 2H), 4.10 (q, 2H), 8.03 (d, 1H), 8.31 (d, 1H), 9.90 (s, 1H).

### Step 3:

Sodium borohydride (80 mg, 2.1 mmol) is added to a solution of trans-(4-{[(5-bromo-3-formylpyridin-2-yl)ethylamino]methyl}cyclohexyl)acetic acid ethyl ester (580 mg, 1.4 mmol) in EtOH (3 mL) at ambient temperature and the mixture is stirred for 3 hours at the same temperature. After adding ammonium chloride solution, the mixture is extracted with AcOEt, washed twice with water, dried and concentrated under reduced pressure to give trans-(4-{[(5-bromo-3-hydroxymethylpyridin-2-yl)ethylamino]methyl}cyclohexyl)acetic acid ethyl ester (0.53 g, 91% yield).

¹H-NMR (400 MHz, CDCl₃), δ (ppm): 0.88-0.97 (m, 4H), 1.15 (t, 3H), 1.24 (t, 3H), 1.45-1.55 (m, 1H), 1.65-1.79 (m, 5H), 2.14 (d, 2H), 3.00 (d, 2H), 3.09 (q, 2H), 3.73 (br, 1H), 4.11 (q, 2H), 4.67 (s, 2H), 7.73 (s, 1H), 8.28 (s, 1H).

### Example E

### Preparation of [3,5-bis(trifluoromethyl)benzyl](2-chloro-5-trifluoromethylpyridin-3-ylmethyl)(2-methyl-2H-tetrazol-5-yl)amine

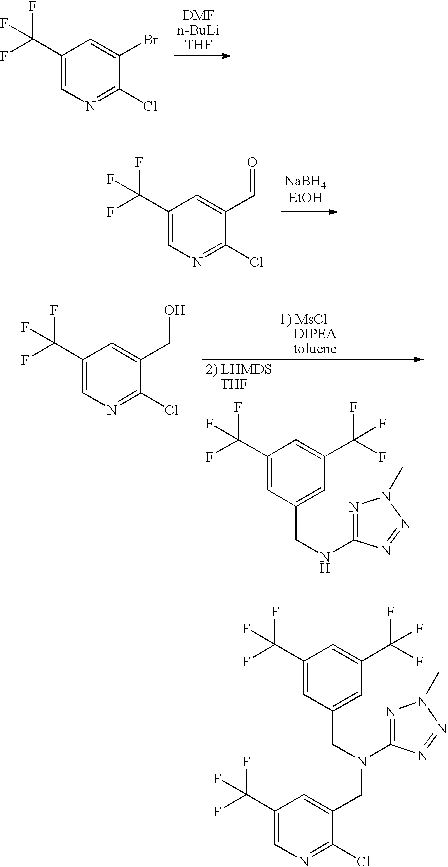

### Step 1:

n-BuLi (1.57M solution in hexane; 64 mL, 0.10 mol) is added dropwise to a solution of 3-bromo-2-chloro-5-trifluoromethylpyridine (20.00 g, 0.077 mol), DMF (7.72 mL, 0.10 mol) in toluene (400 mL) at −65° C. After stirring at the same temperature for 30 min, the mixture is quenched by addition of 1N HCl and extracted with ethyl acetate. The organic layer is washed with water, brine, dried over magnesium sulfate, filtered and concentrated to give crude 2-chloro-5-trifluoromethylpyridine-3-carbardehyde.

To a solution of crude 2-chloro-5-trifluoromethylpyridine-3-carbardehyde in ethanol (60 mL), sodium tetraborohydride (2.90 g, 0.077 mol) is added portionwise and stirred for 30 min at room temperature. After adding sat. ammonium chloride solution, the mixture is extracted with ethyl acetate. The organic layer is washed with sat. ammonium chloride solution, brine, dried over magnesium sulfate, filtered and concentrated. The residue is purified by silica gel column chromatography to give 2-chloro-5-trifluoromethylpyridin-3-ylmethanol (12.3 g, 76%).

### Step 2:

Methanesulfonyl chloride (3.4 mL, 0.044 mol) and N,N-diisopropylethylamine (7.8 mL, 0.045 mol) are added dropwise to a solution of 2-chloro-5-trifluoromethylpyridin-3-ylmethanol (3.72 g g, 0.018 mol) in toluene (90 mL) at 0° C. and the mixture is stirred for 12 hours at room temperature. The mixture is diluted with water, and sat. NaHCO₃ aqueous solution, the mixture is extracted with ethyl acetate. The combined organic layer is washed with brine, dried over magnesium sulfate, filtered and concentrated to give crude 2-chloro-3-chloromethyl-5-trifluoromethylpyridine.

Lithium bis(trimethylsilyl)amide (LHMDS, 1.0M in THF; 25.2 mL, 0.025 mol) is added dropwise to a solution of N-[3,5-bis(trifluoromethyl)phenylmethyl]-N-(2-methyl-2H-tetrazol-5-yl)amine (7.15 g, 0.022 mmol) in THF (60 mL) and the mixture is stirred for 30 min at room temperature. This solution is added dropwise to a solution of crude 2-chloro-3-chloromethyl-5-trifluoromethylpyridine in DMF (60 mL) at −40° C. and the mixture is stirred for 3 hours at same temperature. After warming up to room temperature, the mixture is quenched by addition of sat. ammonium chloride solution and extracted with ethyl acetate twice. The combined organic layer is washed with water, brine, dried over magnesium sulfate, filtered and concentrated. The residue is purified by silica gel column chromatography to give 3,5-bis(trifluoromethyl)benzyl](2-chloro-5-trifluoromethylpyridin-3-ylmethyl)(2-methyl-2H-tetrazol-5-yl)amine (4.21 g, 45%).

### Example F

### Preparation of [3,5-bis(trifluoromethyl)benzyl](2-chloro-5-trifluoromethylpyridin-3-ylmethyl)(2-methyl-2H-tetrazol-5-yl)amine

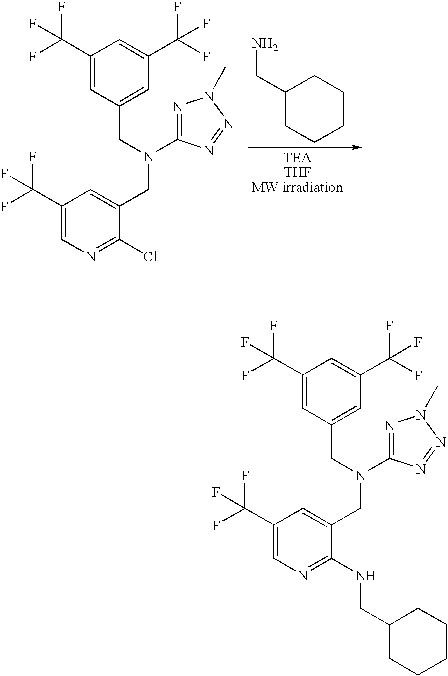

A suspension of 3,5-bis(trifluoromethyl)benzyl](2-chloro-5-trifluoromethylpyridin-3-ylmethyl)(2-methyl-2H-tetrazol-5-yl)amine (0.40 g, 0.77 mmol), cyclohexylmethylamine (0.13 g, 1.1 mmol), triethylamine (0.16 g, 1.6 mmol) in THF (4.0 mL) is irradiated in a microwave reactor for 30 min. After adding water, the mixture is extracted with dichloromethane. The organic layer is filtrated through phase separator and concentrated. The resulting mixture is purified by silica gel column chromatography to give [3,5-bis(trifluoromethyl)benzyl](2-chloro-5-trifluoromethylpyridin-3-ylmethyl)(2-methyl-2H-tetrazol-5-yl)amine (0.34 g, 74%).

### Example G

### Preparation of trans-[2-(ethyl{4-[2-(tetrahydropyran-2-yloxy)ethyl]cyclohexylmethyl}amino)-5-trifluoromethylpyridin-3-yl]methanol

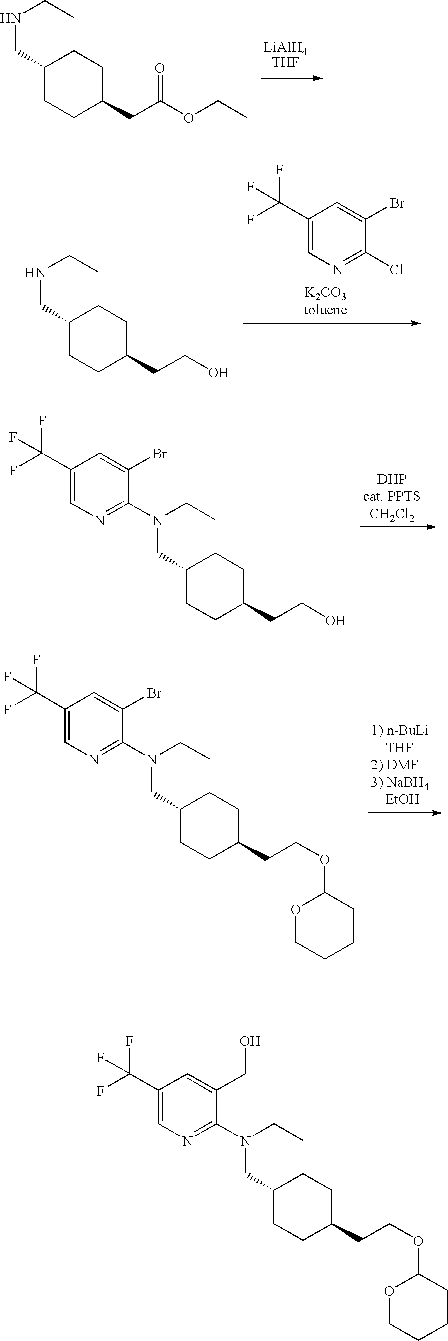

### Step 1:

trans-[4-(Ethylaminomethyl)cyclohexyl)acetic acid ethyl ester (5.5 g, 24 mmol) in THF (50 mL) is added dropwise to a suspension of lithium aluminum hydride (0.92 g, 24 mmol) in THF (15 mL) at 0-13° C. over 20 min and the resulting mixture is stirred at room temperature for 12 hours. After adding sodium sulfate hydrate (Na₂SO₄-10H₂O, 10 g) at 0° C., the mixture is filtered and concentrated in vacuo to give trans-[4(ethylaminomethyl)cyclohexyl]methanol (4.0 g, 89%).

¹H-NMR (400 MHz, CDCl₃), δ (ppm): 0.92-0.99 (m, 4H), 1.10 (t, 3H), 1.36-1.57 (m, 4H), 1.74-1.80 (m, 4H), 2.45 (d, 2H), 2.63 (q, 2H), 3.69 (t, 2H).

### Step 2:

A mixture of 3-bromo-2-chloro-5-trifluoromethylpyridine (0.38 g, 1.5 mmol) and trans-[4-(ethylaminomethyl)cyclohexyl]methanol (0.44 g, 2.4 mol), potassium carbonate (0.66 g, 4.8 mmol) in toluene (3.0 mL) is stirred under reflux condition for 12 hours. After cooling to room temperature, water is added and then the mixture is extracted with ethyl acetate. The combined organic layer is washed with brine, dried over magnesium sulfate, filtrated and concentrated in vacuo. The residue is purified by silica gel column chromatography to afford trans-2-(4-{[(3-bromo-5-trifluoromethylpyridin-2-yl)ethylamino]methyl}cyclohexyl)ethanol (0.21 g, 35% yield).

¹H-NMR (400 MHz, CDCl₃), δ (ppm): 0.86-0.95 (m, 4H), 1.14 (t, 1H), 1.18 (t, 3H), 1.30-1.37 (m, 1H), 1.45 (dt, 2H), 1.55-1.63 (m, 1H), 1.72-1.76 (m, 4H), 3.34 (d, 2H), 3.50 (q, 2H), 3.67 (dt, 2H), 7.89 (m, 1H), 8.37 (m, 1H).

### Step 3:

To a solution of trans-2-(4-{[(3-bromo-5-trifluoromethylpyridin-2-yl)ethylamino]methyl}cyclohexyl)ethanol (0.20 g, 0.49 mmol) in dichloromethane (3.0 mL), dihydropyrane (DHP, 0.10 g, 1.2 mmol) and pyridinium p-toluene sulfonate (12 mg) are added and the mixture is stirred at ambient temperature for 12 hours. After addition of sat. sodium bicarbonate solution, the mixture is partitioned and the organic layer is concentrated in vacuo. The residue is purified by silica gel column chromatography to afford trans-(3-bromo-5-trifluoromethyl-pyridin-2-yl)ethyl{4-[2-(tetrahydropyran-2-yloxy)ethyl]cyclohexylmethyl}amine (0.20 g, 83% yield).

¹H-NMR (400 MHz, CDCl₃), δ (ppm): 0.85-0.92 (m, 4H), 1.18 (t, 3H), 1.28-1.37 (m, 1H), 1.45-1.60 (m, 7H), 1.65-1.84 (m, 6H), 3.34 (d, 2H), 3.36-3.43 (m, 1H), 3.46-3.52 (m, 1H), 3.51 (q, 2H), 3.73-3.78 (m, 1H), 3.79-3.85 (m, 1H), 4.54-4.56 (m, 1H), 7.89 (m, 1H), 8.36 (m, 1H).

### Step 4:

After THF (30 mL) is cooled to −78° C. under argon atmosphere, sec-BuLi (1.0 M in pentane, 18.5 mL) is added over 10 min. A solution of trans-(3-bromo-5-trifluoromethyl-pyridin-2-yl)ethyl{4-[2-(tetrahydropyran-2-yloxy)ethyl]cyclohexylmethyl}amine (3.65 g, 7.4 mmol) in THF (12 mL) is added dropwise over 10 min at −72˜−60° C. After stirring for 1 min, dry DMF is added and the mixture is stirred for 30 min at same temperature. After adding sat. ammonium chloride and then water, the mixture is extracted with ethyl acetate. The combined organic layer is washed with brine, dried over magnesium sulfate, filtrated and concentrated in vacuo to give crude trans-2-(ethyl-{4-[2-(tetrahydropyran-2-yloxy)ethyl]cyclohexylmethyl}amino)-5-trifluoromethylpyridine-3-carbaldehyde.

The obtained crude trans-2-(ethyl-{4-[2-(tetrahydropyran-2-yloxy)ethyl]cyclohexylmethyl}amino)-5-trifluoromethylpyridine-3-carbaldehyde is dissolved in ethanol (40 mL) and sodium tetraborohydride (0.39 g, 10 mmol) is added to the solution. After the mixture is stirred for 2 hours at ambient temperature, sat. ammonium chloride is slowly added and then water and ethyl acetate are added. After partition, the combined organic layer is washed with brine, dried over magnesium sulfate, filtrated and concentrated in vacuo. The residue is purified by silica gel column chromatography to give trans-[2-(ethyl{4-[2-(tetrahydropyran-2-yloxy)ethyl]cyclohexylmethyl}amino)-5-trifluoromethylpyridin-3-yl]methanol (2.25 g, 2 steps 54% yield).

¹H-NMR (400 MHz, CDCl₃), δ (ppm): 0.83-0.93 (m, 4H), 1.10 (t, 3H), 1.28-1.35 (m, 1H), 1.44-1.60 (m, 7H), 1.66-1.84 (m, 6H), 2.68 (t, 1H), 3.17 (d, 2H), 3.26 (q, 2H), 3.36-3.43 (m, 1H), 3.46-3.52 (m, 1H), 3.73-3.79 (m, 1H), 3.82-3.87 (m, 1H), 4.54-4.56 (m, 1H), 4.69 (d, 2H), 7.85 (d, 1H), 8.43 (d, 1H).

### Example H

### Preparation of trans-2-{2-[4-({[3-({[3,5-bis(trifluoromethyl)benzyl](2-methyl-2H-tetrazol-5-yl)amino}methyl)-5-trifluoromethylpyridin-2-yl]ethylamino}methyl)cyclohexyl]ethyl}isoindole-1,3-dione

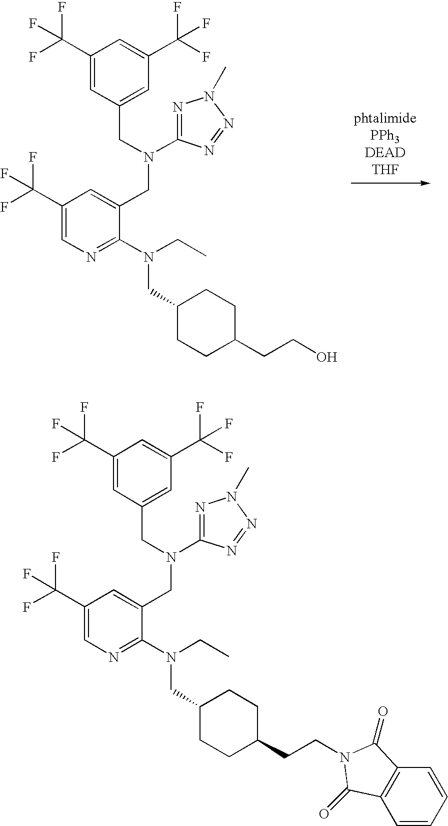

To a solution of trans-2-[4-({[3-({[3,5-bis(trifluoromethyl)benzyl](2-methyl-2H-tetrazol-5-yl)amino}methyl)-5-trifluoromethylpyridin-2-yl]ethylamino}methyl)cyclohexyl]ethanol (0.20 g, 0.30 mmol), phtalimide (0.066 g, 0.45 mmol), triphenylphosphine (0.12 g, 0.46 mmol) in THF (2 mL), diethyl azadicarboxylate (DEAD 40% in toluene, 0.19 g) is added dropwise and the mixture is stirred at ambient temperature for 15 hours. After the mixture is concentrated in vacuo, the residue is purified by silica gel column chromatography to give trans-2-{2-[4-({[3-({[3,5-bis(trifluoromethyl)benzyl](2-methyl-2H-tetrazol-5-yl)amino}methyl)-5-trifluoromethylpyridin-2-yl]ethylamino}methyl)cyclohexyl]ethyl}isoindole-1,3-dione (0.23 g, 96% yield).

¹H-NMR (400 MHz, CDCl₃), δ (ppm): 0.76-0.88 (m, 4H), 1.05 (t, 3H), 1.12-1.20 (m, 1H), 1.50-1.55 (m, 3H), 1.64-1.68 (m, 2H), 1.74-1.79 (m, 2H), 3.08 (d, 2H), 3.16 (q, 2H), 3.67 (t, 2H), 4.22 (s, 3H), 4.61 (s, 2H), 4.68 (s, 2H), 7.48 (d, 1H), 7.59 (s, 2H), 7.70 (dd, 2H), 7.76 (s, 1H), 7.83 (dd, 2H), 8.38 (d, 1H).

### Example I

### Preparation of trans-(4-{[(3-{([3,5-bis(trifluoromethyl)benzylamino]methyl}-5-trifluoromethylpyridin-2-yl)ethylamino]methyl}cyclohexyl)acetic acid ethyl ester

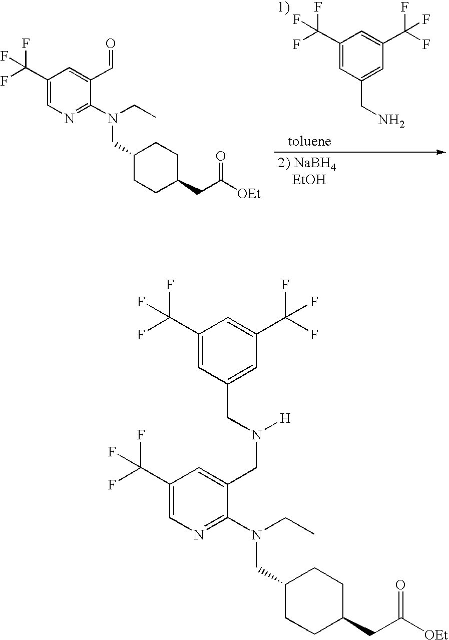

A solution of (4-{[ethyl-(3-formyl-5-trifluoromethylpyridin-2-yl)amino]methyl}cyclohexyl)acetic acid ethyl ester (2.16 g, 6.6 mmol), 3,5-bis(trifluoromethyl)benzylamine (0.96 g) in toluene (30 mL) is stirred under reflux conditions removing water for 3 hours. After cooling to room temperature, the mixture is concentrated in vacuo. The crude residue is dissolved in ethanol (23 mL) and then sodium borohydride (0.17 g) is added portionwise. After stirring at ambient temperature, the mixture is purified by silica gel column chromatography (twice) to give trans-(4-{[(3-{[3,5-bis(trifluoromethyl)benzylamino]methyl}-5-trifluoromethylpyridin-2-yl)ethylamino]methyl}cyclohexyl)acetic acid ethyl ester.

### Example J

### Preparation of (3-{[3,5-bis(trifluoromethyl)benzylamino]methyl}-5-trifluoromethylpyridin-2-yl}(cyclopentylmethyl)ethylamine

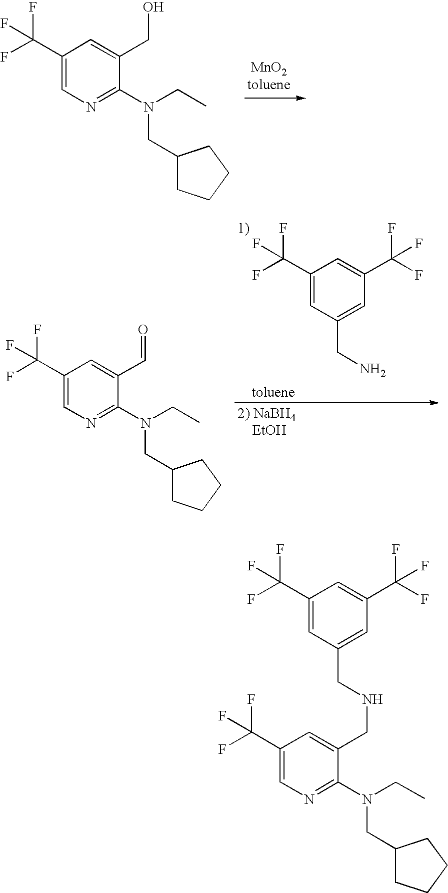

### Step 1:

A suspension of [2-[(cyclopentylmethyl)ethylamino]-5-trifluoromethylpyridin-3-yl]methanol (0.10 g, 0.33 mmol) and manganese(IV)oxide (0.72 g, 8.4 mmol) in toluene is stirred at room temperature for 20 hours. The mixture is filtered and then the residue is washed with ethyl acetate. The filtrate is concentrated to give 2-[(cyclopentylmethyl)ethylamino]-5-trifluoromethylpyridine-3-carbaldehyde (0.087 g, 88%), which is used for the next reaction without further purification.

¹H-NMR (400 MHz, CDCl₃), δ (ppm): 1.09-1.17 (m, 2H), 1.24 (t, 3H), 1.48-1.75 (m, 6H), 2.26-2.34 (m, 1H), 3.55 (d, 2H), 3.60 (q, 2H), 8.13 (m, 1H), 8.49 (m, 1H), 9.91 (s, 1H).

### Step 2:

A suspension of 2-[(cyclopentylmethyl)ethylamino]-5-trifluoromethylpyridine-3-carbaldehyde (0.92 g, 3.1 mmol) and 3,5-bis(trifluoromethyl)benzylamine (1.12 g, 4.6 mmol) in toluene is stirred at 100° C. for 3 hours. After cooling to room temperature, the mixture is concentrated. The crude residue is dissolved with EtOH and the mixture is treated with sodium tetraborohydride (0.12 g, 3.1 mmol). The resulting mixture is stirred at room temperature for 20 hours. After adding sat. ammonium chloride solution, the mixture is extracted with dichloromethane. The organic layer is washed with water and brine, dried over sodium sulfate, filtrated and concentrated in vacuo. The residue is purified by silica gel column chromatography to give (3-{[3,5-bis(trifluoromethyl)benzylamino]methyl}-5-trifluoromethylpyridin-2-yl}(cyclopentylmethyl)ethylamine (0.66 g, 41%).

¹H-NMR (400 MHz, CDCl₃), δ (ppm): 1.06-1.10 (m, 2H), 1.07 (t, 3H), 1.45-1.65 (m, 6H), 2.05-2.13 (m, 1H), 3.24 (d, 2H), 3.28 (q, 2H), 3.80 (s, 2H), 3.87 (s, 2H), 7.78 (s, 1H), 7.80 (s, 2H), 7.85 (d, 1H), 8.41 (d, 1H), 9.91.

### Example K

### Preparation of (R)-2-cyclohexylpyrrolidine

(R)-2-cyclohexylpyrrolidine is prepared using the same procedures for (S)-2-cyclopentylpyrrolidine (see J. Org. Chem., 1992, 57, 1656-1662.) as shown below.

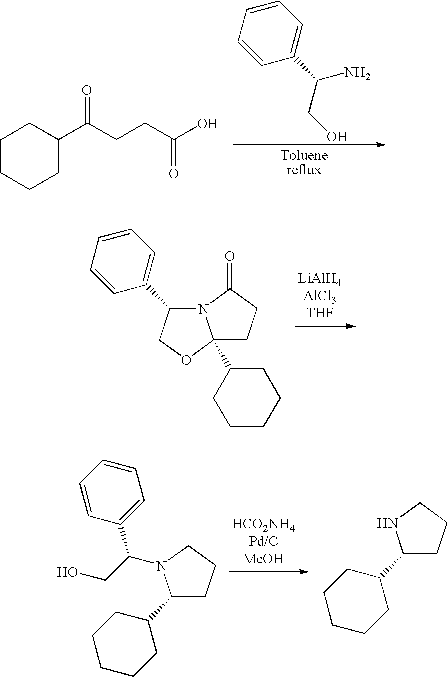

### Example L

### Preparation of trans-(R)-2-[(4-benzyloxymethyl)cyclohexyl]pyrrolidine

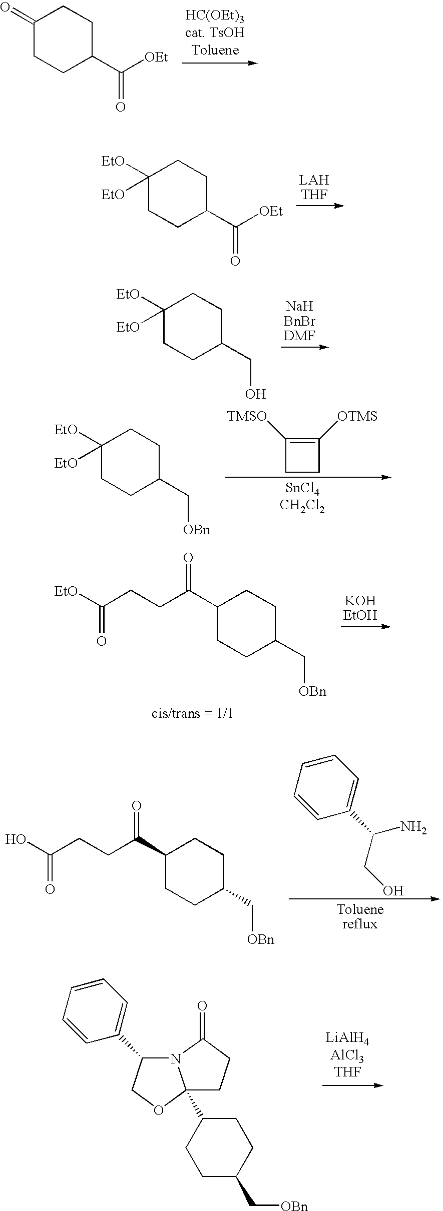

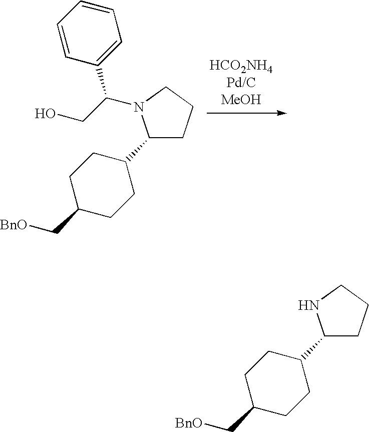

### Step 1:

4-ethoxycarbonyl cyclohexanone (10 g, 58.8 mmol) is dissolved in toluene (150 mL). Triethylorthoformate (39 mL, 235 mmol) and p-toluenesulfonic acid (1.0 g, 5.8 mmol) are added and the resulting mixture is stirred at 130° C. for 3 hours. After addition of triethylamine (1 mL) at room temperature, the mixture is extracted with EtOAc. The water layer is extracted with EtOAc and the combined organic layer is washed with sat. NaHCO₃ aq. and brine, dried over magnesium sulfate, and concentrated under reduced pressure to afford crude 4,4-diethoxycyclohexanecarboxylic acid ethyl ester. The crude product is used without further purification.

### Step 2:

To a solution of lithium aluminum hydride (3.3 g, 88.1 mmol) in THF (80 mL) is carefully added crude 4,4-diethoxycyclohexanecarboxylic acid ethyl ester in THF solution (25 mL) at 0° C. After stirring for 10 min at ambient temperature, Na₂SO₄-10H₂O is added at 0° C. and the mixture is stirred for additional 10 min. Insoluble matter is filtered and the filtrate is concentrated in vacuo to afford crude (4,4-diethoxycyclohexyl)methanol, which is used without further purification.

To a solution of crude (4,4-diethoxycyclohexyl)methanol in DMF (80 mL) is carefully added NaH (60% in oil, 3.5 g, 88 mmol) at room temperature and the mixture is stirred for 15 min at the same temperature. To the mixture is dropped benzyl bromide (10.5 mL, 88.1 mmol) at room temperature and stirring is continued for 30 min at the same temperature. After addition of H₂O, the mixture is extracted with EtOAc. The water layer is extracted with EtOAc and the combined organic layer is washed with brine, dried over magnesium sulfate, and concentrated under reduced pressure. The crude mixture is purified by silica gel column chromatography (hexane/EtOAc=10/1) to afford (4,4-diethoxycyclohexylmethoxymethyl)benzene.

¹H-NMR (400 MHz, CDCl₃), δ (ppm): 1.21 (m, 8H), 1.38 (m, 2H), 1.67 (m, 3H), 2.01 (m, 2H), 3.30 (d, 2H), 3.40 (q, 2H), 3.50 (q, 2H), 4.49 (s, 2H), 7.29 (m, 5H).

### Step 3:

To a solution of tin tetrachloride (1.1 mL, 10 mmol) in CH₂Cl₂ (30 mL) are added (4,4-diethoxycyclohexylmethoxymethyl)benzene (3.3 g, 10 mmol) and 1,2-bis(trimethylsiloxy)cyclobutene (3.0 mL, 11 mmol) in CH₂Cl₂ (18 mL) at −70° C. by cannula. The mixture is stirred for 10 min at −70° C. and 15 min at −40° C. After addition of H₂O, the mixture is extracted with EtOAc. The water layer is extracted with EtOAc and the combined organic layer is washed with 1N HCl, sat. NaHCO₃ aq. and brine, dried over magnesium sulfate, and concentrated under reduced pressure. The crude mixture is purified by silica gel column chromatography (hexane/EtOAc=8/1) to afford 4-(4-benzyloxycyclohexyl)-4-oxobutyric acid (cis/trans=1/1).

¹H-NMR (400 MHz, CDCl₃), δ (ppm): 1.03 (dq, 1H), 1.14 (t, 3H), 1.37 (m, 2H), 1.59 (m, 2H), 1.80-1.96 (m, 4H), 2.35 (m, 0.5H), 2.54 (m, 2.5H), 2.74 (m, 2H), 3.29 (dd, 2H), 4.09 (q, 2H), 4.49 (s, 2H), 7.29 (m, 5H).

### Step 4:

Potassium hydroxide (4.24 g, 76 mmol) is added to an EtOH (50 mL) solution of 4-(4-benzyloxycyclohexyl)-4-oxobutyric acid (cis/trans=1/1, 5.0 g, 15 mmol), and the mixture is stirred at 80° C. for 5 hours. After addition of 5N HCl (to reach pH 3-4) at 0° C., the mixture is extracted with EtOAc. The water layer is extracted with EtOAc and the combined organic layer is washed with water and brine, dried over magnesium sulfate, and concentrated under reduced pressure to afford trans-4-{[4-(2-benzyloxy)methyl]cyclohexyl}-4-oxobutyric acid.

¹H-NMR (400 MHz, CDCl₃), δ (ppm): 0.97-1.10 (m, 2H), 1.31-1.45 (m, 3H), 1.56-1.68 (m, 1H), 1.89-1.99 (m, 4H), 2.30-2.38 (m, 1H), 2.62 (t, 2H), 2.77 (t, 2H), 3.29 (d, 2H), 4.49 (s, 2H), 7.26-7.40 (m, 5H).

### Step 5:

To a stirred solution of (S)-(+)-phenylglycinol (1.8 g, 13 mmol) in toluene (40 mL) is added trans-4-{[4-(2-benzyloxy)methyl]cyclohexyl}-4-oxobutyric acid (4.0 g, 13 mmol) and the resulting solution is heated to reflux for 7 hours. The resulting solution is cooled to room temperature and then water and EtOAc are added to the solution. The water layer is extracted with EtOAc, and combined organic layer is washed with water, brine, dried over magnesium sulfate, filtered and concentrated. The crude residue is purified by silica gel column chromatography to afford trans-(3S,7aS)-7a-[4-(benzyloxymethyl)cyclohexyl]-3-phenyltetrahydropyrrolo[2,1-b]oxazol-5-one.

Rf value: 0.44 (Hexane/EtOAc=3/1)

¹H-NMR (400 MHz, CDCl₃), δ (ppm): 0.73-0.89 (m, 2H), 1.08-1.21 (m, 2H), 1.45-1.68 (m, 2H), 1.83-2.06 (m, 5H), 2.40-2.46 (m, 1H), 2.58 (ddd, 1H), 2.75 (dt, 1H), 3.23 (d, 2H), 4.07 (dd, 1H), 4.46 (s, 2H), 4.65 (t, 1H), 5.19 (t, 1H), 7.21 (d, 2H), 7.25-7.38 (m, 8H).

### Step 6:

To a cooled (0° C.) quantity of anhydrous AlCl₃ (1.47 g, 11 mmol) is added THF (70 mL) via syringe under a static nitrogen atmosphere. The resulting solution is allowed to stir at 0° C. for 5 min, and lithium aluminum hydride (1.0 M in THF, 36 mL) solution is added via syringe, and the mixture is stirred at the same temperature for 20 min. To a stirred, cooled (−78° C.) solution of the resulting THF solution is added a solution of trans-(3S,7aS)-7a-[4-(benzyloxymethyl)cyclohexyl]-3-phenyltetrahydropyrrolo[2,1-b]oxazol-5-one (4.97 g, 12 mmol) in THF (100 mL) via syringe, and the resulting solution is stirred at the same temperature for 2 hours, and then warmed to room temperature and stirred for an additional 1 hour. The resulting solution is recooled to 0° C. and quenched with careful addition of 1N HCl via syringe and extracted with CH₂Cl₂ 3 times. Combined organic layers are washed with 1N NaOH, brine, dried over magnesium sulfate, filtered and concentrated. The crude residue is purified by silica gel column chromatography to afford trans-(S)-2-{(R)-2-[4-benzyloxymethyl)cyclohexyl]pyrrolidin-1-yl}-2-phenylethanol.

¹H-NMR (400 MHz, CDCl₃), δ (ppm): 0.95-1.16 (m, 4H), 1.40-1.66 (m, 6H), 1.67-1.76 (m, 1H), 1.77-1.85 (m, 1H), 1.86-1.94 (m, 2H), 2.20-2.38 (m, 1H), 2.58-2.66 (m, 1H), 2.87-2.95 (m, 1H), 3.31 (dd, 2H), 3.59-3.65 (m, 1H), 3.72-3.77 (m, 1H), 3.96-4.04 (m, 2H), 4.51 (s, 2H), 7.15-7.17 (m, 2H), 7.25-7.49 (m, 8H).

### Step 7:

To a stirred solution of anhydrous ammonium formate (17.2 g, 0.27 mol) and trans-(S)-2-{(R)-2-[4-benzyloxymethyl)cyclohexyl]pyrrolidin-1-yl}-2-phenylethanol (4.75 g, 0.012 mol) in MeOH (200 mL) is added 10% palladium on carbon (7.5 g). The resulting mixture is stirred at room temperature under an argon atmosphere for 3 hours. The reaction mixture is filtered and the filtrate is concentrated. The residue is dissolved with 1N HCl and extracted with ether to remove phenethylalcohol. The water layer is neutrized by addition of 1 N NaOH, and extracted with CH₂Cl₂ 3 times. Combined organic layer is washed with brine, dried over magnesium sulfate, filtered and concentrated to give trans-(R)-2-[4-benzyloxymethyl)cyclohexyl]pyrrolidine. The crude product is used without further purification.

Rf value: 0.14 (CH₂Cl₂/MeOH=9/1)

¹H-NMR (400 MHz, CDCl₃), δ (ppm): 0.89-1.06 (m, 4H), 1.12-1.21 (m, 1H), 1.23-1.35 (m, 1H), 1.46-1.78 (m, 4H), 1.82-1.90 (m, 4H), 1.95-2.03 (m, 1H), 2.64 (q, 1H), 2.80-2.90 (m, 1H), 2.97-3.03 (m, 1H), 3.27 (d, 2H), 4.49 (s, 2H), 7.22-7.37 (m, 5H).

### Example M

### Preparation of trans-2-{(R)-2-[4-(2-benzyloxyethyl)cyclohexyl]pyrrolidin-1-yl}-5-trifluoromethylpyridine-3-carboxaldehyde

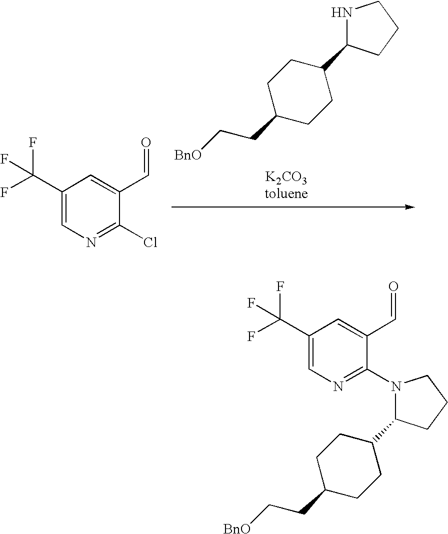

A mixture of 2-chloro-5-trifluoromethylpyridine-3-carboxaldehyde (330 mg, 1.6 mmol), trans-(R)-2-[4-(benzyloxyethyl)cyclohexyl]pyrrolidine (410 mg, 1.5 mmol), potassium carbonate (310 mg. 2.2 mmol) in toluene (3.5 mL) is stirred under reflux condition for 5 hours. After cooling to room temperature, water and dichloromethane are added and the mixture is extracted with dichloromethane. The combined organic layer is filtered through phase separator and concentrated. The residue is purified by silica gel column chromatography to give trans-2-{(R)-2-[4-(2-benzyloxyethyl)cyclohexyl]pyrrolidin-1-yl}-5-trifluoromethylpyridine-3-carboxaldehyde (527 mg).

0.82-1.23 (m, 4H), 1.69-1.55 (m, 4H), 1.81-1.91 (m, 3H), 1.92-2.11 (m, 3H), 2.98-3.03 (m, 1H), 3.23 (d, 2H), 3.65-3.72 (m, 1H), 4.48 (s, 2H), 4.52-4.70 (m, 1H), 7.32 (s, 1H), 7.25-7.37 (m, 5H), 8.11 (d, 1H), 8.50 (d, 1H), 9.93 (s, 1H).

Rf value: 0.41 (Hexane/EtOAc=9/1)

### Example N

### Preparation of trans-(4-{(R)-1-[3-({[3,5-bis(trifluoromethyl)benzyl](5-morpholin-4-yl-pyrimidin-2-yl)amino}methyl)-5-trifluoromethylpyridin-2-yl]pyrrolidin-2-yl}cyclohexyl)methanol

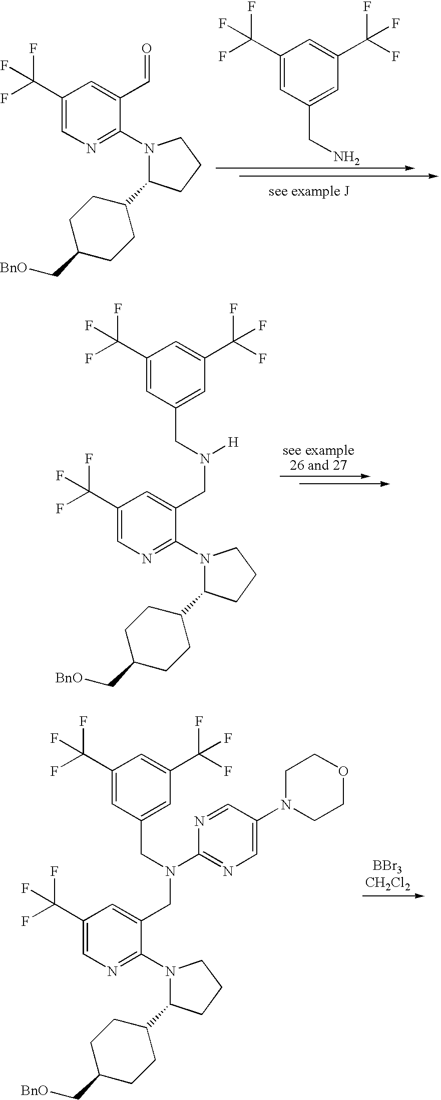

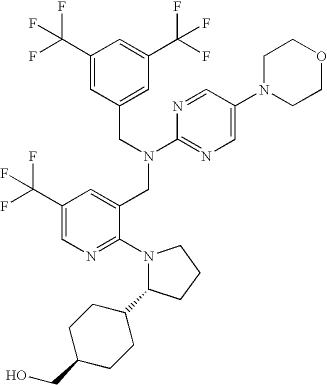

### Step 1

trans-(2-{(R)-2-[4-(benzyloxymethyl)cyclohexyl]pyrrolidin-1-yl}-5-trifluoromethylpyridin-3-ylmethyl)[3,5-bis(trifluoromethyl)benzyl](5-morpholin-4-yl-pyrimidin-2-yl)amine is prepared from 3-bromo-2-chloro-5-trifluoromethylpyridine, trans-(R)-2-[4-benzyloxymethyl)cyclohexyl]pyrrolidine and corresponding reagents following the procedures of example C, J, 26, and 27.

¹H-NMR (400 MHz, CDCl₃), δ (ppm): 0.78-1.14 (m, 4H), 1.50-1.62 (m, 2H), 1.63-1.76 (m, 4H), 1.77-1.91 (m, 3H), 1.94-2.04 (m, 1H), 3.05-3.08 (m, 4H), 3.20-3.29 (m, 3H), 3.46-3.57 (m, 1H), 3.87 (t, 4H), 4.34 (d, 1H), 4.47 (s, 2H), 4.45-4.55 (m, 1H), 4.57 (d, 1H), 5.01 (d, 1H), 5.26 (d, 1H), 7.24-7.37 (m, 6H), 7.63 (s, 2H), 7.74 (s, 1H), 8.13 (s, 2H), 8.28 (s, 1H).

Rf value: 0.27 (Hexane/EtOAc=9/1)

### Step 2:

To a stirred solution of trans-(2-{(R)-2-[4-(Benzyloxymethyl)cyclohexyl]pyrrolidin-1-yl}-5-trifluoromethylpyridin-3-ylmethyl)[3,5-bis(trifluoromethyl)benzyl](5-morpholin-4-yl-pyrimidin-2-yl)amine (0.48 g, 0.57 mol) in CH₂Cl₂ (5.0 mL) is added dropwise BBr₃ (1.0 M CH₂Cl₂ solution, 0.69 mL, 0.69 mmol) at 0° C., and the mixture is stirred at room temperature for 2 hours. The reaction mixture is quenched by addition of water and extracted with CH₂Cl₂. Combined organic layer is filtered through phase separator and concentrated. The crude product is purified by silica gel column chromatography to give of trans-(4-{(R)-1-[3-({[3,5-bis(trifluoromethyl)benzyl](5-morpholin-4-yl-pyrimidin-2-yl)amino}methyl)-5-trifluoromethylpyridin-2-yl]pyrrolidin-2-yl}cyclohexyl)methanol (304 mg).

¹H-NMR (400 MHz, CDCl₃), δ (ppm): 0.75-1.14 (m, 4H), 0.19-1.29 (m, 1H), 1.35-1.47 (m, 1H), 1.50-1.83 (m, 7H), 1.84-1.92 (m, 1H), 1.94-2.04 (m, 1H), 3.06-3.08 (m, 4H), 3.20-3.28 (m, 1H), 3.41 (t, 2H), 3.46-3.55 (m, 1H), 3.86-3.89 (m, 4H), 4.33 (d, 1H), 4.48-4.56 (m, 1H), 4.56 (d, 1H), 5.02 (d, 1H), 5.28 (d, 1H), 7.31 (s, 1H), 7.63 (s, 2H), 7.74 (s, 1H), 8.14 (s, 2H), 8.29 (s, 1H).

Rf value: 0.20 (Hexane/EtOAc=2/1)

### Example O

### Preparation of trans-(R)-2-[4-(2-benzyloxyethyl)cyclohexyl]pyrrolidine

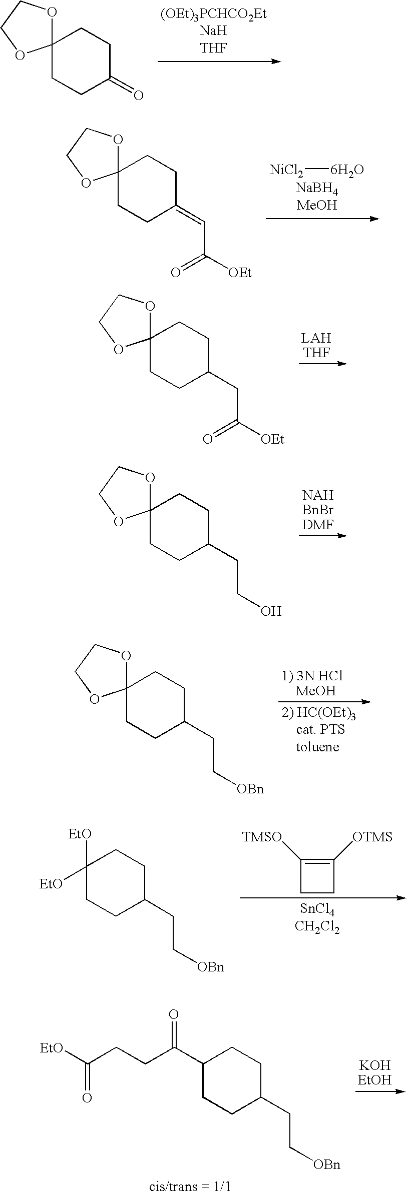

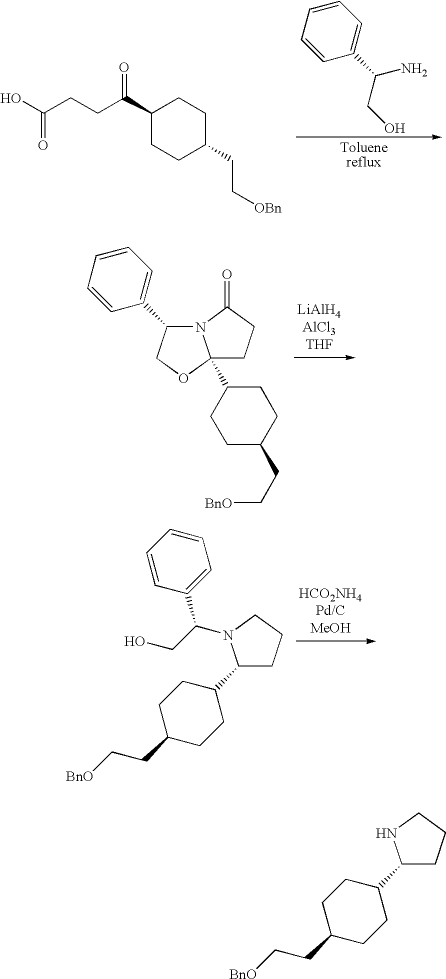

### Step 1:

Triethylphosphonoacetate (14 mL, 70.6 mmol) is added to a suspension of NaH (60% in oil, 2.8 g, 70.8 mmol) in THF (270 mL) at 0° C. and the mixture is stirred for 30 min at the same temperature. To the mixture is added dropwise 1,4-cyclohexanedione monoethylene acetal (10 g, 64.4 mmol) in THF (65 mL) at 0° C. and stirring is continued for 40 min at the same temperature. After addition of H₂O, the mixture is extracted with EtOAc. The water layer is extracted with EtOAc and combined organic layer is washed with brine, dried over magnesium sulfate, and concentrated under reduced pressure. The crude mixture is purified by silica gel column chromatography (hexane/EtOAc=9/1) to afford 8-ethoxycarbonylmethylidene-1,4-dioxaspiro[4.5]decane.

¹H-NMR (400 MHz, CDCl₃), δ (ppm): 1.27 (t, 3H), 1.76 (m, 4H), 2.37 (t, 2H), 3.00 (t, 2H), 3.98 (s, 4H), 4.15 (q, 2H), 5.66 (s, 1H).

### Step 2:

To a solution of 8-ethoxycarbonylmethylidene-1,4-dioxaspiro[4.5]decane (380 mg, 1.68 mmol) and nickel dichloride hexahydrate (40 mg, 0.16 mmol) in MeOH (3 mL) is carefully added sodium borohydride (450 mg, 11.8 mmol) at 0° C. After stirring for 10 min, H₂O is added and the mixture is extracted with EtOAc. The water layer is extracted with EtOAc and the combined organic layer is washed with brine, dried over magnesium sulfate, and concentrated under reduced pressure to afford crude 8-ethoxycarbonylmethyl-1,4-dioxaspiro[4.5]decane.

¹H-NMR (400 MHz, CDCl₃), δ (ppm): 1.25 (t, 3H), 1.32 (m, 2H), 1.55 (td, 2H), 1.74 (m, 4H), 1.84 (m, 1H), 2.20 (d, 2H), 3.94 (s, 4H), 4.12 (q, 2H).

### Step 3:

To a solution of lithium aluminum hydride (110 mg, 2.80 mmol) in THF (3 mL) is carefully added crude 8-ethoxycarbonylmethyl-1,4-dioxaspiro[4.5]decane (320 mg) in THF solution (2 mL) at 0° C. After stirring for 10 min at ambient temperature, Na₂SO₄-10H₂O is added at 0° C. and the mixture is stirred for additional 10 min. Insoluble matter is filtered and the filtrate is concentrated in vacuo to afford crude 2-(1,4-dioxaspiro[4.5]dec-8-yl)ethanol.

¹H-NMR (400 MHz, CDCl₃), δ (ppm): 1.28 (m, 2H), 1.48 (m, 5H), 1.64 (bs, 1H), 1.74 (m, 4H), 3.69 (t, 2H), 3.94 (s, 4H).

### Step 4:

To a solution of crude 2-(1,4-dioxaspiro[4.5]dec-8-yl)ethanol (180 mg) in DMF (4 mL) is carefully added NaH (60% in oil, 80 mg, 1.93 mmol) at room temperature and the mixture is stirred for 15 min at the same temperature. To the mixture is added dropwise benzyl bromide 230 μL, 1.93 mmol) at room temperature and stirring is continued for 30 min at the same temperature. After addition of H₂O, the mixture is extracted with EtOAc. The water layer is extracted with EtOAc and the combined organic layer is washed with brine, dried over magnesium sulfate, and concentrated under reduced pressure. The crude mixture is purified by silica gel column chromatography (hexane/EtOAc=5/1) to afford 8-[(2-(benzyloxy)ethyl]-1,4-dioxaspiro[4.5]decane.

¹H-NMR (400 MHz, CDCl₃), δ (ppm): 1.25 (m, 2H), 1.52 (m, 5H), 1.74 (m, 4H), 3.50 (t, 2H), 3.93 (s, 4H), 4.49 (s, 2H), 7.34 (m, 5H).

### Step 5:

To a solution of 8-[2-(benzyloxy)ethyl]-1,4-dioxaspiro[4.5]decane (270 mg, 0.97 mmol) in THF (3 mL) is added 3N HCl (3 mL) at room temperature and the mixture is stirred for 3 hours at the same temperature. After addition of sat. NaHCO₃ aq., the mixture is extracted with EtOAc. The water layer is extracted with EtOAc and the combined organic layer is washed with brine, dried over magnesium sulfate, and concentrated under reduced pressure.

The resulting crude material is dissolved in toluene (7 mL), and then triethylorthoformate (1.5 mL, 6.83 mmol) and p-toluenesulfonic acid (20 mg, 0.10 mmol) are added. The resulting mixture is stirred at 130° C. for 3 hours. After addition of triethylamine (1 mL) at room temperature, the mixture is extracted with EtOAc. The water layer is extracted with EtOAc and the combined organic layer is washed with sat. NaHCO₃ aq. and brine, dried over magnesium sulfate, and concentrated under reduced pressure. The crude mixture is purified by silica gel column chromatography (hexane/EtOAc=10/1) to afford [2-(4,4-diethoxycyclohexyl)ethoxymethyl]benzene.

¹H-NMR (400 MHz, CDCl₃), δ (ppm): 1.14 (t, 6H), 1.16-1.40 (m, 5H), 1.59 (m, 4H), 1.99 (m, 2H), 3.39 (q, 2H), 4.48 (q, 2H), 4.50 (s, 2H), 7.34 (m, 5H).

### Step 6:

To a solution of tin tetrachloride (1.1 mL, 10 mmol) in CH₂Cl₂ (30 mL) are added by cannula a solution of [2-(4,4-diethoxycyclohexyl)ethoxymethyl]benzene (3.3 g, 10 mmol) and 1,2-bis(trimethylsiloxy)cyclobutene (3.0 mL, 11 mmol) in CH₂Cl₂ (18 mL) at −70° C. The mixture is stirred for 10 min at −70° C. and 15 min at −40° C. After addition of H₂O, the mixture is extracted with EtOAc. The organic layer is extracted with EtOAc and the combined organic layer is washed with 1N HCl, sat. NaHCO₃ aq. and brine, dried over magnesium sulfate, and concentrated under reduced pressure to afford crude ethyl trans-4-{[4-(2-benzyloxy)ethyl]cyclohexyl}-4-oxobutyrate.

Potassium hydroxide (1.5 g, 26 mmol) is added to an EtOH (30 mL) solution of crude ethyl trans-4-{[4-(2-benzyloxy)ethyl]cyclohexyl}-4-oxobutyrate and the resulting mixture is stirred at 80° C. for 3 hours. After addition of 5N HCl (to reach pH 34) at 0° C., the mixture is extracted with EtOAc. The water layer is extracted with EtOAc and the combined organic layer is washed with 1N HCl, sat. NaHCO₃ aq. and brine, dried over magnesium sulfate, and concentrated under reduced pressure to obtain brown solid. The solid is suspended in Et₂O (5 mL) and hexane (3 mL), and collected by filtration to afford trans-4-{[4-(2-benzyloxy)ethyl]cyclohexyl}-4-oxobutyric acid.

¹H-NMR (400 MHz, CDCl₃), δ (ppm): 0.96 (q, 2H), 1.35 (m, 3H), 1.41 (q, 2H), 1.83 (m, 4H), 2.32 (m, 1H), 2.62 (t, 2H), 2.76 (t, 2H), 3.49 (m, 2H), 7.35 (m, 5H).

### Step 7:

To a stirred solution of (S)-(+)-phenylglycinol (215 mg, 1.6 mmol) in toluene (5.0 mL) is added trans-4-{[4-(2-benzyloxy)ethyl]cyclohexyl}-4-oxobutyric acid (0.50 g, 1.6 mmol). The resulting solution is heated to reflux for 5 hours. The resulting solution is cooled to room temperature and then water and CH₂Cl₂ are added to the solution. The organic layer is filtrated by phase separator and concentrated. The crude residue is purified by silica gel column chromatography to afford trans-(3S,7aS)-7a-[4-(benzyloxyethyl)cyclohexyl]-3-phenyltetrahydropyrrolo[2,1-b]oxazol-5-one.

Rf value: 0.48 (Hexane/EtOAc=3/1)

¹H-NMR (400 MHz, CDCl₃), δ (ppm): 0.67-0.84 (m, 2H), 1.06-1.21 (m, 2H), 1.30-1.52 (m, 4H), 1.73-2.04 (m, 5H), 2.40-2.46 (m, 1H), 2.58 (ddd, 1H), 2.75 (dt, 1H), 3.47 (t, 2H), 4.07 (dd, 1H), 4.48 (s, 2H), 4.64 (t, 1H), 5.19 (t, 1H), 7.21 (d, 2H), 7.24-7.36 (m, 8H).

### Step 8:

To a cooled (0° C.) quantity of anhydrous AlCl₃ (157 mg, 1.2 mmol) is added THF (7.0 mL) via syringe under a static nitrogen atmosphere. The resulting solution is allowed to stir at 0° C. for 5 min, and lithium aluminum hydride (1.0 M in THF, 3.8 mL) solution is added via syringe, and the mixture is stirred at the same temperature for 20 min. To a stirred, cooled (−78° C.) solution of the resulting THF solution is added a solution of trans-(3S,7aS)-7a-[4-(benzyloxyethyl)cyclohexyl]-3-phenyltetrahydropyrrolo[2,1-b]oxazol-5-one (537 mg, 1.3 mmol) in THF (10 mL) via syringe, and the resulting solution is stirred at the same temperature for 1 hour, and then warmed to room temperature and stirred for additional 1 hour. The resulting solution is cooled to 0° C. and quenched with careful addition of 1N HCl via syringe and extracted with CH₂Cl₂ 3 times. Combined organic layer is washed with 1N NaOH, brine, dried over magnesium sulfate, filtered and concentrated. The crude residue is purified by silica gel column chromatography to afford trans-(S)-2-{(R)-2-[4-(benzyloxyethyl)cyclohexyl]pyrrolidin-1-yl}-2-phenylethanol.

Rf value: 0.32 (Hexane/EtOAc=3/1)

¹H-NMR (400 MHz, CDCl₃), δ (ppm): 0.88-1.16 (m, 4H), 1.35-1.90 (m, 12H), 2.19-2.29 (m, 1H), 2.58-2.66 (m, 1H), 2.84-2.94 (m, 1H), 3.52 (t, 2H), 3.60-3.66 (m, 1H), 3.72-3.77 (m, 1H), 3.95-4.04 (m, 2H), 4.51 (s, 2H), 7.16 (d, 2H), 7.28-7.52 (m, 8H).

### Step 9:

To a stirred solution of anhydrous ammonium formate (3.24 g, 0.051 mmol) and trans-(S)-2-{(R)-2-[4-(benzyloxyethyl)cyclohexyl]pyrrolidin-1-yl}-2-phenylethanol (0.90 g, 2.2 mmol) in MeOH (27 mL) is added 10% palladium on carbon (765 mg). The resulting mixture is stirred at room temperature under an argon atmosphere for 3 hours. The reaction mixture is filtered and the filtrate is concentrated. The residue is dissolved with 1N HCl and the mixture is extracted with ether to remove phenethylalcohol. The water layer is neutrized by addition of 1N NaOH, and extracted with CH₂Cl₂ 3 times. Combined organic layer is washed with brine, dried over magnesium sulfate, filtered and concentrated to give trans-(R)-2-[4-(benzyloxyethyl)cyclohexyl]pyrrolidine. The crude product is used without further purification.

¹H-NMR (400 MHz, CDCl₃), δ (ppm): 0.86-1.07 (m, 4H), 1.11-1.18 (m, 1H), 1.24-1.43 (m, 3H), 1.47-1.64 (m, 2H), 1.66-1.97 (m, 7H), 2.62 (q, 1H), 2.78-2.90 (m, 1H), 2.97-3.02 (m, 1H), 3.50 (t, 2H), 4.49 (s, 2H), 7.22-7.37 (m, 5H).

### Example P

### Preparation of trans-2-(4-{(R)-1-[3-({[3,5-bis(trifluoromethyl)benzyl](2-methyl-2H-tetrazol-5-yl)amino}methyl)-5-trifluoromethylpyridin-2-yl]pyrrolidin-2-yl}cyclohexyl)ethanol

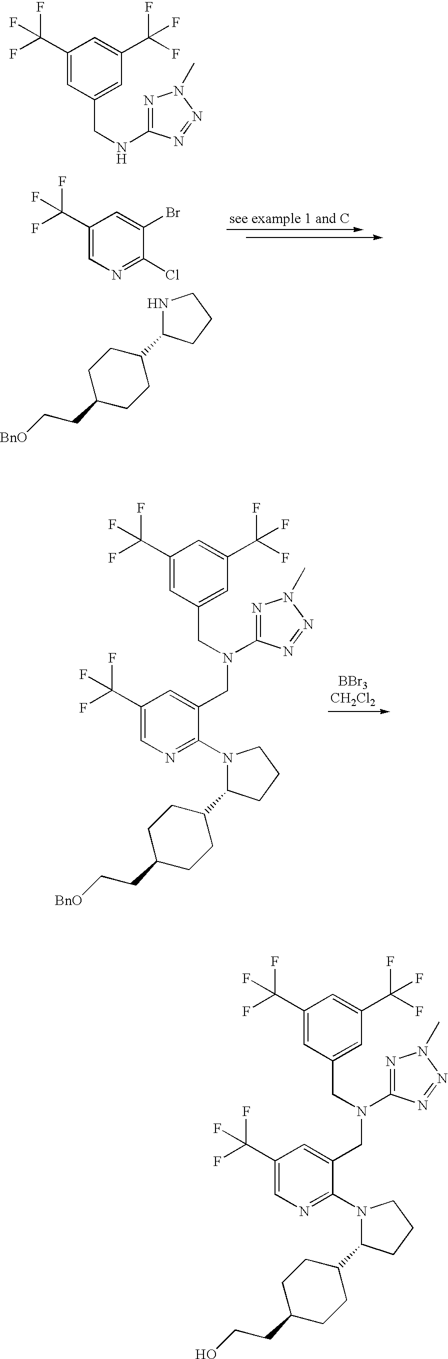

### Step 1

trans-(2-{(R)-2-[4-(2-benzyloxyethyl)cyclohexyl]-pyrrolidin-1-yl}-5-trifluoromethylpyridin-3-ylmethyl)[3,5-bis(trifluoromethyl)benzyl](2-methyl-2H-tetrazol-5-yl)amine is prepared from 3-bromo-2-chloro-5-trifluoromethylpyridine, [3,5-bis(trifluoromethyl)benzyl](2-methyl-2H-tetrazol-5-yl)amine, and trans-(R)-2-[4-(benzyloxyethyl)cyclohexyl]pyrrolidine following the procedure of example 1 and C.

ESI-MS m/z: 770 [M+1]⁺

### Step 2:

To a stirred solution of trans-(2-{(R)-2-[4-(2-benzyloxyethyl)cyclohexyl]-pyrrolidin-1-yl}-5-trifluoromethylpyridin-3-ylmethyl)[3,5-bis(trifluoromethyl)benzyl](2-methyl-2H-tetrazol-5-yl)amine (0.13 g, 0.17 mmol) in CH₂Cl₂ (1.0 mL) is added dropwise BBr₃ (1.0 M CH₂Cl₂ solution, 0.30 mL, 0.30 mmol) at 0° C., and the mixture is stirred at room temperature for 2 hours. The reaction mixture is quenched by addition of water and extracted with CH₂Cl₂. Combined organic layer is filtered through phase separator and concentrated. The crude product is purified by silica gel column chromatography to give trans-2-(4-{(R)-1-[3-({[3,5-bis(trifluoromethyl)benzyl](2-methyl-2H-tetrazol-5-yl)amino}methyl)-6,7-difluoroquinolin-2-yl]pyrrolidin-2-yl}cyclohexyl)ethanol (61 mg).

¹H-NMR (400 MHz, CDCl₃), δ (ppm): 0.73-1.14 (m, 4H), 1.24-1.36 (m, 1H), 1.40-1.52 (m, 3H), 1.55-1.79 (m, 7H), 1.82-2.03 (m, 2H), 3.16-3.21 (m, 1H), 3.47-3.53 (m, 1H), 3.66 (t, 2H), 4.21 (s, 3H), 4.46 (d, 1H), 4.45-4.50 (m, 1H), 4.56 (d, 1H), 4.68 (d, 1H), 4.89 (d, 1H), 7.38 (d, 1H), 7.60 (s, 2H), 7.76 (s, 1H), 8.30 (d, 1H).

Rf value: 0.10 (Hexane/EtOAc=5/1)

### Example Q

### Preparation of ethyl[(tetrahydropyran-4-yl)methyl)]amine

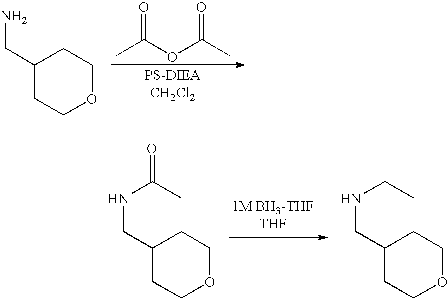

### Step 1:

PS-DIEA (Argonaut Technologies, 1.35 g, 4.5 mmol) is added to a solution of C-(tetrahydropyran-4-yl)methylamine (345 mg, 3.0 mmol) in CH₂Cl₂ (20 ml) at ambient temperature. Acetic anhydride (367 mg, 3.6 mmol) is added to the mixture. After stirring at ambient temperature for 18 hours, methylisocyanate polystyrene (Novabiochem, 1.84 g, 3.0 mmol) and N-(2-aminoethyl)aminomethyl polystyrene (Novabiochem, 1.07 g, 3.0 mmol) are added. After stirring at room temperature for 4 h, the resins are removed by filtration, and the resins are washed with dichloromethane. The filtrate and washing are combined, and the solvent is removed by evaporation in vacuo to give N-(tetrahydropyran-4-ylmethyl)acetamide.

ESI-MS m/z: 158 [M+1]⁺

HPLC retention time: 0.94 min.

### Step 2:

1M Borane-THF complex solution in THF (10.2 ml, 10.2 mmol) is added to a solution of N-(tetrahydropyran-4-ylmethyl)acetamide (235 mg, 1.50 mmol) in THF (15 ml) at ambient temperature under nitrogen gas atmosphere. After stirring for 2 days, methanol (5 ml) is added to the reaction mixture at ambient temperature. After stirring for 1 hour, 1N HCl (50 ml) is added to the solution, and a part of THF is removed by evaporation in vacuo. The solution is washed with ether and 5N NaOH is added to the solution. The product is extracted with CH₂Cl₂, and the organic phase is washed with brine, dried over magnesium sulfate, and concentrated to give N-ethyl-N—[(tetrahydropyran-4-yl)methyl)]amine.

ESI-MS m/z: 144 [M+1]⁺

HPLC retention time: 0.58 min.

### Example R

### Preparation of trans-ethyl(4-methoxycyclohexylmethyl)amine

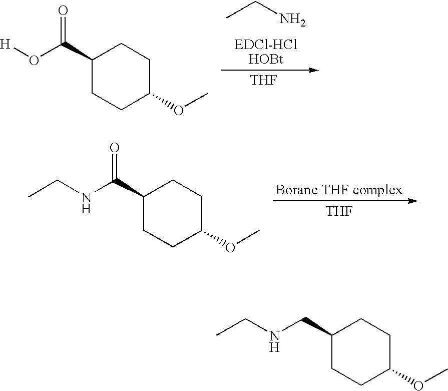

### Step 1:

A mixture of trans-4-methoxycyclohexanecarboxylic acid (290 mg, 1.84 mmol), 2M ethylamine solution in THF (3.67 mL, 7.34 mmol), 1-hydroxybenzotriazole (370 mg, 2.74 mmol), and N-(3-dimethylaminopropyl)-N-ethylcarbodiimide hydrochloride (523 mg, 2.74 mmol) in DMF (4 mL) is stirred for 18 hours at ambient temperature. After addition of 0.1N HCl aqueous solution, the mixture is extracted with dichloromethane. The organic layer is washed with 0.1N HCl aqueous solution, sat. NaHCO₃ aqueous solution, and brine, dried over magnesium sulfate, filtrated and concentrated to give trans-4-methoxycyclohexanecarboxylic acid ethylamide (40 mg)

ESI-MS m/z: 186 [M+1]⁺. UPLC retention time: 1.27 min.

### Step 2:

To a stirred solution of trans-4-methoxycyclohexanecarboxylic acid ethylamide (40 mg, 0.22 mmol) in THF (6.5 mL), 1M borane-THF complex solution in THF (0.65 mL, 0.65 mmol) is added at ambient temperature under nitrogen atmosphere. The mixture is stirred for 18 h and then methanol is added. The mixture is stirred for 1 h and then 1N HCl aqueous solution (5 mL) is added. A part of the solvent is removed by evaporation. The residue is washed with ether and 5N NaOH aqueous solution (5 mL) is added. The mixture is extracted with dichloromethane and the organic layer is washed with water and brine, dried over sodium sulfate and concentrated in vacuo. The crude product, trans-ethyl(4-methoxycyclohexylmethyl)amine is directly used without further purification.

ESI-MS m/z: 172 [M+1]⁺. UPLC retention time: 1.13 min.

### Example S

### Preparation of 4-(ethylamino)cyclohexanecarboxylic acid ethyl ester

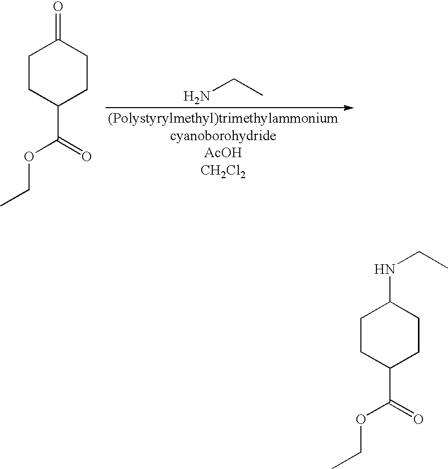

A mixture of 4-oxo-cyclohexanecarboxylic acid ethyl ester (1.0 g, 5.9 mmol), 2N ethylamine solution in THF (5.9 mL, 11.8 mmol) in AcOH-dichloromethane [1:20, 21 mL] is stirred for 15 minutes at ambient temperature. (Polystyrylmethyl)trimethylammonium cyanoborohydride (5.7 g, 23.4 mmol) is added to the solution. The mixture is stirred for 18 hours at ambient temperature. The resin is removed by filtration and the filtrate is concentrated in vacuo. To the residue, 1N HCl aqueous solution (5 mL) is added, and the solution is washed with diethyl ether. 5N NaOH aqueous solution (5 mL) is added to the solution, and the mixture is extracted with dichloromethane and the organic layer is washed with brine, dried over magnesium sulfate and concentrated in vacuo to give 4-(ethylamino)cyclohexanecarboxylic acid ethyl ester (550 mg), which is used without further purification.

ESI-MS m/z: 200 [M+1]⁺. UPLC retention time: 1.28 min.

### Example T

### Preparation of N-[3,5-bis(trifluoromethyl)benzyl]-N-{2-[2-(tetrahydropyran-2-yloxy)ethyl]-2H-tetrazol-5-yl}amine

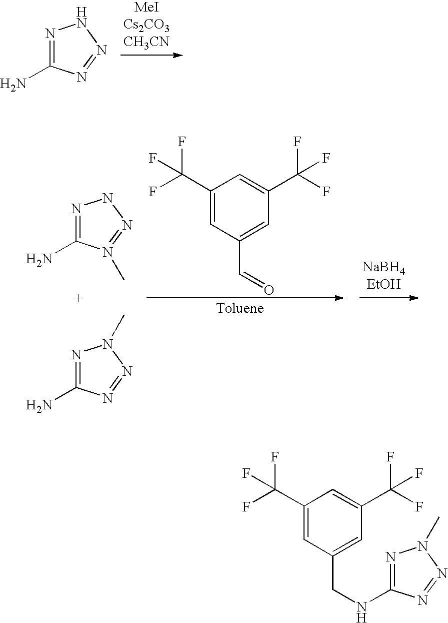

A mixture of 5-aminotetrazole (24.4 g, 0.29 mol), methyliodide (48.8 g, 0.34 mol), and Cs₂CO₃ (112.0 g, 0.34 mol) in acetonitrile (700 mL) is stirred and refluxed for 7 hours. The mixture is cooled to 50° C. and filtrated. The resulting filtrate is concentrated to give a mixture of 5-amino-2-methyltetrazole and 5-amino-1-methyltetrazole.

A mixture of the crude product and 3,5-bis(trifluoromethyl)benzaldehyde (43.0 g, 0.18 mol) in toluene (600 mL) is stirred and refluxed for 45 min. After cooling to room temperature, the resulting mixture is concentrated. NaBH₄ (8.12 g, 0.22 mol) is added portionwise slowly to EtOH (500 mL) solution of the resulting residue, and the mixture is stirred at room temperature for 4 hours. After addition of sat. NH₄Cl aq. and water, the mixture is extracted with ethyl acetate. The combined organic layer is washed with brine, dried over magnesium sulfate, filtered and concentrated. The crude product is purified by crystallization (50 mL of i-PrOH: H₂O. 3:7) to give [3,5-bis(trifluoromethyl)phenylmethyl](2-methyl-2H-tetrazol-5-yl)amine (12.4 g).

### General UPLC Condition

Column: Waters ACQUITY UPLC BEH C18, 1.7 μM

Mobile phase: CH₃CN/H₂O (0.1% TFA)

1. A compound of formula I 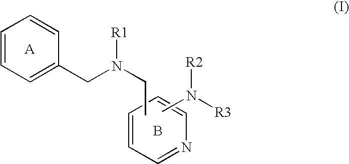
wherein
Z₁ is selected from the group consisting of —N(R₂)(R₃), —CN, —OR′, —COR′, —C(═O)—O—R′, —C(═O)—NR₂R₃, —S(O)ₘR′, —S(O)ₘ—N(R₂)(R₃) and —NR′—S(O)ₘ—N(R₂)(R₃), m being in each case the integer 0, 1 or 2, or Z₁ is Z;
R₁ is the element C(═O)—R′, —C(═O)—O—R′, —C(═O)—NR₂R₃, —S(O)ₘ—R′, —S(O)ₘ—N(R₂)(R₃), m being in each case the integer 0, 1 or 2, or R₁ is Z;
wherein, in each case, independently of one another,
Z is selected from the group consisting of (i) unsubstituted or substituted monocyclic cycloalkyl or unsubstituted or substituted monocyclic cycloalkenyl, (ii) unsubstituted or substituted carbocyclic aromatic radical or unsubstituted or substituted heterocyclic, radical;
R′, independently, represents hydrogen, alkyl, haloalkyl, unsubstituted or substituted cycloalkyl, unsubstituted or substituted cycloalkenyl, in the cycloalkyl moiety unsubstituted or substituted cycloalkylalkyl, in the cycloalkenyl moiety unsubstituted or substituted cycloalkenyl-alkyl, unsubstituted or substituted carbocyclic aromatic radical, unsubstituted or substituted heterocyclic radical or in the aryl moiety unsubstituted or substituted aralkyl;
R₂ and R₃, independently of one another, represents hydrogen, alkyl, alkyl which is substituted by one or more substituents selected from the group consisting of halogen, hydroxy, —N(R₂)(R₃), —C(═O)—O—R′, —C(═O)—NR₂R₃, —S(O)ₘ—R′, —S(O)ₘ—N(R₂)(R₃), unsubstituted or substituted cycloalkyl, unsubstituted or substituted cycloalkenyl, and unsubstituted or substituted heterocyclic radical; or R₂ and R₃, independently of one another, represents unsubstituted or substituted cycloalkyl, unsubstituted or substituted cycloalkenyl, or unsubstituted or substituted carbocyclic aromatic radical, of unsubstituted or substituted heterocyclic radical; and
R₂ and R₃ together are unsubstituted or substituted alkylene or unsubstituted or substituted alkylene that is interrupted by O, NR″ or S; R″ being R′ or —C(═O)—O—R′; and
wherein substituted cycloalkyl or substituted cycloalkenyl each of which substituted is by one or more substituents selected from the group consisting of alkyl, of alkoxy, of —C(═O)—O—R′, of —C(═O)—NR₂R₃, of N(R₂)(R₃), of cycloalkyl-alkyl, of unsubstituted or substituted carbocyclic aromatic radical, of unsubstituted or substituted heterocyclic radical, of in the aryl moiety unsubstituted or substituted aralkyl, and of in the heterocyclyl moiety unsubstituted or substituted heterocyclyl-alkyl; and
wherein a carbocyclic aromatic radical or a heterocyclic aromatic radical or a heterocyclic radical, in the aryl moiety unsubstituted or substituted aralkyl, in the heterocyclyl moiety unsubstituted or substituted heterocycyl-alkyl, or the rings A and B, independently of one another, are unsubstituted or substituted by one or more substituents selected from the group consisting of halogen, NO₂, CN, OH, alkyl, alkoxy-alkyl, hydroxy-alkyl, halo-alkyl, alkoxy, alkoxy-alkoxy, haloalkoxy, —C(═O)—R′, —C(═O)—O—R′, —N(R₂)(R₃), —C(═O)—NR₂R₃, —S(O)ₘ—R′, —S(O)ₘ—N(R₂)(R₃), —NR′—S(O)ₘ—N(R₂)(R₃) and alkanoyl(oxy), m being in each case the integer 0, 1 or 2; and unsubstituted or substituted cycloalkyl, unsubstituted or substituted cycloalkenyl; in the aryl moiety unsubstituted or substituted aralkyl and in the heterocyclyl moiety unsubstituted or substituted heterocyclyl-alkyl;
in free form or in salt form.

2. The compound according to claim 1 of formula (I′) 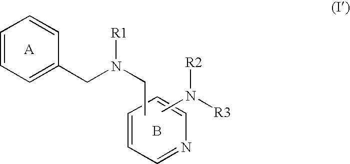
or a pharmaceutically acceptable salt thereof,
wherein R₁ is carbocyclic or heterocyclic aryl, alkoxy-CO—, cylcoalkyl-alkoxy-CO—, carbocyclic aryl-alkoxy-CO—, alkyl-S(O)₂—, cycloalkyl-alkyl-S(O)₂—, carbocyclic aryl-alkyl-S(O)₂— or hetero-carbocyclic aryl-alkyl-S(O)₂—;
R₂ or R₃, independently of one another represent alkyl, cycloalkyl-alkyl cycloalkyl being unsubstituted or substituted by alkyl or by carboxy-alkyl, by alkoxy-CO-alkyl or by carbocyclic aryl-alkoxy-CO-alkyl, or represent carbocyclic or heterocyclic arylalkyl, alkoxy-CO-alkyl or by carbocyclic-aryl-alkoxy-CO-alkyl; or
R₂ and R₃ together represent C₂-C₈-alkylene;
wherein ring A and ring B, independent of one another, or carbocyclic or heterocyclic aryl, is otherwise unsubstituted or substituted by a substituent selected from the group consisting of halogen, NO₂, CN, OH, alkyl, alkoxy-alkyl, halo-alkyl, alkoxy, alkoxy-alkoxy, alkyl-S(O)ₙ, cycloalkyl-alkyl-S(O)ₙ, carbocyclic or heterocyclic aryl-alkyl-S(O)ₙ, n being in each case the integer 0, 1 or 2, halo-alkoxy, carbocyclic or heterocyclic aryl, and alkanoyl(oxy), and wherein two substituents together with the two carbon atoms to which they are attached can form a 5 or 6-membered ring which can be unsubstituted or otherwise substituted by a substitutent selected from the group as specified above.

3. The compound according to claim 1 of formula (I′) 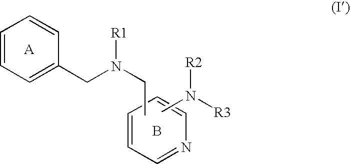
wherein R₁ is carbocyclic or heterocyclic aryl, alkoxy-CO—, cylcoalkyl-alkoxy-CO—, carbocyclic aryl-alkoxy-CO—, alkyl-S(O)₂—, cycloalkyl-alkyl-S(O)₂—, carbocyclic aryl-alkyl-S(O)₂— or hetero-carbocyclic aryl-alkyl-S(O)₂—;
R₂ or R₃, independently of one another represent alkyl, cycloalkylalkyl cycloalkyl being unsubstituted or substituted by alkyl or by carboxyalkyl, by alkoxy-CO-alkyl or by carbocyclic aryl-alkoxy-CO-alkyl, or represent carbocyclic or heterocyclic aryl-alkyl, alkoxy-CO-alkyl or by carbocyclic aryl-alkoxy-CC-alkyl; or
R₂ and R₃ together represent C₂-C₈-alkylene;
wherein ring A and ring B, independent of one another, or carbocyclic or heterocyclic aryl, is otherwise unsubstituted or substituted by a substituent selected from the group consisting of halogen, NO₂, CN, OH, alkyl, alkoxy-alkyl, halo-alkyl, alkoxy, alkoxy-alkoxy, alkyl-S(O)ᵣ, cycloalkyl-alkyl-S(O)ₙ, carbocyclic or heterocyclic aryl-alkyl-S(O)ₙ, n being in each case the integer 0, 1 or 2, halo-alkoxy, carbocyclic or heterocyclic aryl, and alkanoyl(oxy), and wherein two substituents together with the two carbon atoms to which they are attached can form a 5 or 6-membered ring which can be unsubstituted or otherwise substituted by a substitutent selected from the group as specified above;
in free form or in salt form.

4. The compound according to claim 1 represented by formula (I A) 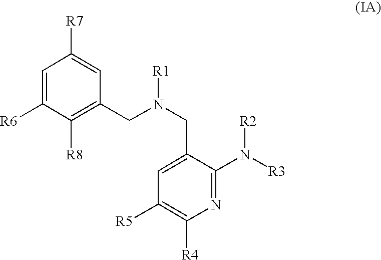
wherein R₁ is 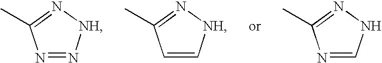
being in each case unsubstituted or N-substituted by a substituent selected from the group consisting of C₁-C₇-alkyl, C₃-C₇-cycloalkyl-C₁-C₇-alkyl, and phenyl-C₁-C₇-alkyl;
or is phenyl, phenacyl, phenyl-S(O)₂, C₂-C₇alkoxycarbonyl, C₂-C₇-alkoxy-thiocarbonyl, carbamoyl, C₁-C₇-alkyl-alkylamino-carbonyl, di-C₁-C₇-alkyl-alkylamino-carbonyl, or C₁-C₇-alkyl-S(O)₂;
R₂ and R₃, independently of one another, represent C₁-C₇-alkyl, C₃-C₇-cycloalkyl-C₁-C₇-alkyl cycloalkyl being unsubstituted or substituted by a substituent selected from the group consisting of C₁-C₇-alkyl, of carboxy-C₁-C₇-alkyl, of C₁-C₇-alkoxycarbonyl-C₁-C₇-alkyl, of carbamoyl-C₁-C₄-alkyl, of C₁-C₇-alkyl-carbamoyl-C₁-C₄-alkyl, of di-C₁-C₇-alkyl-carbamoyl-C₁-C₄-alkyl, of hydroxyl-C₁-C₄-alkyl, of amino-C₁-C₄-alkyl, or represent phenyl-C₁-C₇alkyl, naphthyl-C₁-C₇alkyl, pyridyl-C₁-C₇-alkyl, or C₂-C₇-alkoxycarbonyl; or
R₂ and R₃ together represent C₂-C₆-alkylene being unsubstituted or substituted by a substituent selected from the group consisting of C₁-C₇-alkyl, C₃-C₈-cycloalkyl, and heterocyclyl;
R₄, R₅, R₆, R₇, and R₈, independently of one another, represent hydrogen, halogen, NO₂, CN, OH, C₁-C₇-alkyl, phenyl-C₁-C₇alkyl, naphthyl-C₁-C₇alkyl, pyridyl-C₁-C₇-alkyl, C₃-C₇-cycloalkyl-C₁-C₇-alkyl, C₁-C₇-alkoxy-C₁-C₇-alkyl, phenyl-C₁-C₇-alkoxy, naphthyl-C₁-C₇-alkoxy, pyridyl-C₁-C₇-alkoxy, C₃-C₇-cycloalkyl-C₁-C₇-alkoxy, halo-C₁-C₇-alkyl, C₁-C₇-alkoxy, C₁-C₇-alkoxy-C₁-C₇-alkoxy, C₁-C₇-alkyl-S(O)ₙ—, phenyl-C₁-C₇-alkyl-S(O)ₙ, naphthyl-C₁-C₇-alkyl-S(O)ₙ, pyridyl-C₁-C₇-alkyl-S(O)ₙ, halo-C₁-C₇-alkoxy, phenyl, naphthyl, pyridyl, and C₂-C₇-alkanoyl(oxy);
where, in each case, n is the integer 0, 1 or 2; a phenyl, biphenyl, naphthyl or pyridyl substituent is, independently of one another is unsubstituted or substituted by a substitutent selected from the group consisting of the substituents specified under variables R₄, R₅, R₆, and R₇; or a pharmaceutically acceptable salt thereof.

5. The compound according to claim 1 represented by formula (I A) 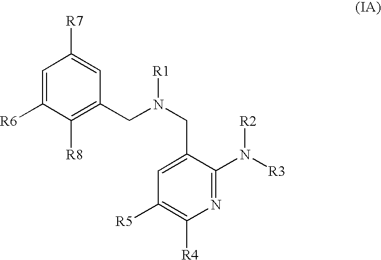
wherein
R₁ is 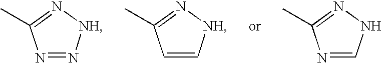
being in each case N-substituted by C₁-C₇-alkyl, C₃-C₇-cycloalkyl-C₁-C₇-alkyl, and phenyl-C₁-C₇-alkyl; or
R₁ is phenyl, formyl, phenacyl, phenyl-S(O)₂, carboxy, C₂-C₇-alkoxycarbonyl, carbamoyl, C₁-C₇-alkyl-alkylamino-carbonyl, di-C₁-C₇-alkyl-alkylamino-carbonyl, or C₁-C₇alkyl-S(O)₂;
R₂ and R₃, independently of one another, represents phenyl, pyridyl, C₁-C₇-alkyl, C₁-C₇-alkanoyl, C₁-C₇-alkyl which is substituted by C₃-C₇cycloalkyl, whereby C₃-C₇cycloalkyl itself is unsubstituted or substituted by C₁-C₇-alkyl (which itself is unsubstituted or substituted by hydroxyl, amino, carboxy, C₁-C₇-alkoxy-carbonyl, carbamoyl, or carbamoyl which is mono- or di substituted by C₁-C₇-alkyl), or represents C₃-C₇-cycloalkyl which is unsubstituted or substituted by C₁-C₇-alkyl, C₃-C₇-cycloalkyl which is interrupted by O and which is unsubstituted or substituted by C₁-C₇-alkyl, or C₃-C₇-cycloalkyl which is interrupted by NH which is unsubstituted or N-substituted by C₁-C₇alkyl, hydroxy-C₁-C₇-alkyl or amino-C₁-C₇-alkyl;
R₂ and R₃ together represent C₂-C₇-alkylene which is unsubstituted or substituted by C₁-C₇-alkyl, C₁-C₇-alkyl which is substituted by C₁-C₇-alkyl, C₁-C₇-alkoxy-C₁-C₇-alkyl carboxy, C₁-C₇-alkoxy-carbonyl, C₃-C₇-cycloalkyl or by phenyl, or represent C₂-C₇-alkylene which is interrupted by O or N—C₁-C₇-alkyl; or represent C₂-C₇-alkylene to which a C₃-C₇-cycloalkyl is either annelated or attached to in spiro form; and
R₄, R₅, R₆, R₇, and R₈, independently of one another, represent hydrogen, halogen, NO₂, CN, halo-C₁-C₇-alkyl, phenyl or pyridyl;
or a pharmaceutically acceptable salt thereof.

6. The compound according to claim 1 represented by formula (I B) 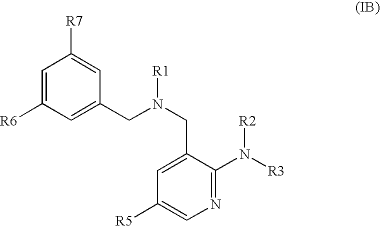
wherein R₁ is 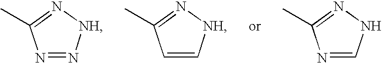
being in each case unsubstituted or N-substituted by C₁-C₇-alkyl;
or is C₂-C₇-alkoxycarbonyl or C₁-C₇-alkyl-S(O)₂;
R₂ is C₁-C₇-alkyl;
R₃ is C₃-C₇-cycloalkyl-C₁-C₇-alkyl cycloalkyl being unsubstituted or substituted by a substituent selected from the group consisting of C₁-C₇-alkyl and of carboxycarbonyl-C₁-C₇-alkyl; or
R₄ is halo-C₁-C₇-alkyl, especially trifluoromethyl;
R₅ is hydrogen;
R₆ is halo-C₁-C₇-alkyl, especially trifluoromethyl; and
R₇ is halogen, NO₂, CN, or halo-C₁-C₇-alkyl, especially trifluoromethyl;
or a pharmaceutically acceptable salt thereof.

7. (canceled)

8. A pharmaceutical composition, comprising:
compound according to claim 1 and
a pharmaceutically acceptable salt thereof and a pharmaceutically acceptable carrier.

9. The pharmaceutical composition of claim 8, further comprising an active principles selected from the group consisting of a:
(i) HMG-CO-A reductase inhibitor or a pharmaceutically acceptable salt thereof,
(ii) angiotensin II receptor antagonist or a pharmaceutically acceptable salt thereof,
(iii) angiotensin converting enzyme (ACE) Inhibitor or a pharmaceutically acceptable salt thereof,
(iv) calcium channel blocker or a pharmaceutically acceptable salt thereof,
(v) aldosterone synthase inhibitor or a pharmaceutically acceptable salt thereof,
(vi) aldosterone antagonist or a pharmaceutically acceptable salt thereof,
(vii) dual angiotensin converting enzyme/neutral endopeptidase (ACE/NEP) inhibitor or a pharmaceutically acceptable salt thereof,
(viii) endothelin antagonist or a pharmaceutically acceptable salt thereof,
(ix) renin inhibitor or a pharmaceutically acceptable salt thereof,
(x) diuretic or a pharmaceutically acceptable salt thereof, and
(xi) an ApoA-I mimic.

10. A method for treating diseases in which CETP is involved, comprising:
administering a therapeutically effective amount to a patient in need thereof the compound according to claim 1.

11. The method according to claim 10, wherein the diseases in which CETP is involved are hyperlipidemia, arteriosclerosis, atherosclerosis, peripheral vascular disease, dyslipidemia, hyperbetalipoproteinemia, hypoalphalipoproteinemia, hypercholesterolemia, hypertriglyceridemia, familial hypercholesterolemia, cardiovascular disorder, coronary heart disease, coronary artery disease, coronary vascular disease, angina, ischemia, heart ischemia, thrombosis, cardiac infarction such as myocardial infarction, stroke, peripheral vascular disease, reperfusion injury, angioplasty restenosis, hypertension, congestive heart failure, diabetes such as type II diabetes mellitus, diabetic vascular complications, obesity or endotoxemia.
